(19) United States
(12) Patent Application Publication
(10) Pub. No.: US 2025/0257345 A1
(43) Pub. Date: Aug. 14, 2025

(54) DAS181 VARIANT COMPOSITIONS

(71) Applicant: Ansun Biopharma, Inc., San Diego, CA (US)

(72) Inventors: George WANG, San Diego, CA (US); Stephen HAWLEY, San Diego, CA (US); Tiejun LI, San Diego, CA (US)

(21) Appl. No.: 18/706,357

(22) PCT Filed: Nov. 1, 2022
(86) PCT No.: PCT/US2022/079087

§ 371 Date: Apr. 30, 2024

Related U.S. Application Data
(60) Provisional application No. 63/263,441, filed on Nov. 2, 2021.

(51) Int. Cl. C12N 9/24; A61K 9/19; A61K 38/00; A61K 47/02; A61K 47/12; A61K 47/26; C07K 1/18

(52) U.S. Cl. CPC C12N 9/2402; A61K 9/19; A61K 47/02; A61K 47/12; A61K 47/26; C07K 1/18; A61K 38/00; C12Y 302/01018

(57) ABSTRACT

The present application provides compositions comprising at least four subspecies of DAS181 that can be separated by CEX-HPLC, methods of releasing a DAS181 composition for human medical use, comprising subjecting the composition to CEX-HPLC, and determining the relative amounts of the first, second, third, and fourth and optionally fifth subspecies separated by the CEX-HPLC, formulations for DAS181 or DAS181 multi¬subspecies compositions, and methods of treating a disease comprising administering any of the DAS181 compositions described herein.

### CROSS-REFERENCE TO RELATED APPLICATIONS

This application claims the benefit of, and priority to, U.S. Provisional Application 63/263,441, filed on Nov. 2, 2021, the contents of which are hereby incorporated herein by reference in their entirety.

### SEQUENCE LISTING

The contents of the electronic sequence listing (208712000440seqlist.xml; Size: 5,657 bytes; and Date of Creation: Oct. 20, 2022) is herein incorporated by reference in its entirety.

### FIELD

The present application relates to compositions comprising subspecies of DAS181 and methods of using same.

### BACKGROUND

DAS181 is a recombinant sialidase protein that cleaves sialic acid, such as sialic acid located on the surface of epithelial cells lining the human respiratory tract. Many different viruses use sialic acid as a receptor for infecting the epithelial cells, and treatment with DAS181 can therefore block virus entry and prevent viral infection and spread. DAS181 has demonstrated anti-viral activity against multiple sialic acid-dependent viruses, which makes it uniquely suitable as a broad-spectrum treatment for many different respiratory viral infections.

Various compositions comprising DAS181 are described in U.S. Pat. Nos. 8,084,036; 7,807,174; 9,764,007; 7,645,448; 8,152,710; 8,722,869; 10,525,109; U.S. 2015/0132274; U.S. Pat. Nos. 9,700,602; and 10,351,828, the contents of each of which are incorporated herein by reference in their entireties.

### BRIEF SUMMARY

The present application provides compositions comprising variants of DAS181 and methods of releasing a composition comprising the DAS181 variants.

In some aspects, provided herein is a composition comprising at least four subspecies of DAS181 that can be separated by cation exchange high performance liquid chromatography (CEX-HPLC), wherein the composition comprises: a) a first subspecies comprising a first fusion protein comprising a sialidase domain fused via its C-terminus to a cationic domain, wherein the cationic domain comprises amino acids 395-415 of SEQ ID NO: 1; b) a second subspecies comprising a deaminated form of the first fusion protein, wherein the deaminated form of the first fusion protein comprises a deaminated N residue compared to the first fusion protein, and wherein the amino acid position is relative to SEQ ID NO: 1; c) a third subspecies comprising a second fusion protein comprising a sialidase domain fused via its C-terminus to a truncated cationic domain, wherein the truncated cationic domain comprises amino acids 395-397 of SEQ ID NO: 1; and d) a fourth subspecies comprising a dimerized form of the first fusion protein.

In some aspects, provided herein is a composition comprising at least four subspecies of DAS181, wherein the composition comprises: a) a first subspecies comprising a first fusion protein comprising a sialidase domain fused via its C-terminus to a cationic domain, wherein the cationic domain comprises amino acids 395-415 of SEQ ID NO: 1; b) a second subspecies comprising a deaminated form of the first fusion protein, wherein the deaminated form of the first fusion protein comprises a deaminated N residue compared to the first fusion protein, and wherein the amino acid position is relative to SEQ ID NO: 1; c) a third subspecies comprising a second fusion protein comprising a sialidase domain fused via its C-terminus to a truncated cationic domain, wherein the truncated cationic domain comprises amino acids 395-397 of SEQ ID NO: 1; and d) a fourth subspecies comprising a dimerized form of the first fusion protein.

In some embodiments, the deaminated N residue in the second subspecies is N403.

In some embodiments according to any of the preceding compositions, the fourth subspecies comprises a methionine oxidized form of the first fusion protein.

In some embodiments according to any of the preceding compositions, the composition further comprises a fifth subspecies comprising a dehydrated form of the first fusion protein.

In some embodiments according to any of the preceding compositions, the proteins in the fourth subspecies have at least about 40% sialidase activity comparing to that of the first fusion protein.

In some embodiments according to any of the preceding compositions, the proteins in the third subspecies have at least about 80% sialidase activity comparing to that of the first fusion protein.

In some embodiments according to any of the preceding compositions, the third subspecies further comprises oxidized or deaminated forms of the second fusion protein.

In some embodiments according to any of the preceding compositions, the proteins in the first subspecies have at least about 94% sialidase activity comparing to that of the first fusion protein.

In some embodiments according to any of the preceding compositions, the first subspecies further comprises a third fusion protein that lacks the N-terminal M residue comparing to the first fusion protein.

In some embodiments according to any of the preceding compositions, the ratio of the first fusion protein and the third fusion protein in the first subspecies is about 2:1.

In some embodiments according to any of the preceding compositions, the proteins in the second subspecies have at least about 80% sialidase activity comparing to that of the first fusion protein.

In some embodiments according to any of the preceding compositions, the sialidase domain comprises amino acids 1-394 of SEQ ID NO: 1.

In some embodiments according to any of the preceding compositions, the first fusion protein comprises amino acids 1-415 of SEQ ID NO: 1.

In some embodiments according to any of the preceding compositions, the second fusion protein comprises amino acids 1-406 of SEQ ID NO: 1.

In some embodiments according to any of the preceding compositions, wherein the weight percentage of the first subspecies among the total proteins in the composition is about 50% to about 90%.

In some embodiments according to any of the preceding compositions, the percentage of the second subspecies among the total proteins in the composition is about 10% to about 30%.

In some embodiments according to any of the preceding compositions, the weight percentage of the third subspecies among the total proteins in the composition is about 1% to about 15%.

In some embodiments according to any of the preceding compositions, the weight percentage of the fourth subspecies among the total proteins in the composition is about 0.1% to about 4%.

In some embodiments according to any of the preceding compositions, the weight percentage of the fifth subspecies among the total proteins in the composition is about 1% to about 10%.

In some embodiments according to any of the preceding compositions, when the composition is subject to cation-exchange chromatography (CEX-HPLC) using non-porous 3 μm polystyrene divinylbenzene bead derivatized with carboxylic acid and elution by a gradient of up to 200 mM CaCl₂) in acetic buffer, the first subspecies elutes at about 80-85 mM CaCl₂).

In some embodiments according to any of the preceding compositions, when the composition is subject to cation-exchange chromatography (CEX-HPLC) using non-porous 3 μm polystyrene divinylbenzene bead derivatized with carboxylic acid and elution by a gradient of up to 200 mM CaCl₂) in acetic buffer, the second subspecies elutes at about 70-75 mM CaCl₂).

In some embodiments according to any of the preceding compositions, the composition is subject to cation-exchange chromatography (CEX-HPLC) using non-porous 3 μm polystyrene divinylbenzene bead derivatized with carboxylic acid and elution by a gradient of up to 200 mM CaCl₂) in acetic buffer, the third subspecies elutes at about 57-58 mM CaCl₂).

In some embodiments according to any of the preceding compositions, when the composition is subject to cation-exchange chromatography (CEX-HPLC) using non-porous 3 μm polystyrene divinylbenzene bead derivatized with carboxylic acid and elution by a gradient of up to 200 mM CaCl₂) in acetic buffer, the fourth subspecies elutes at about 180-200 mM CaCl₂).

In some embodiments according to any of the preceding compositions, when the composition is subject to cation-exchange chromatography (CEX-HPLC) using non-porous 3 μm polystyrene divinylbenzene bead derivatized with carboxylic acid and elution by a gradient of up to 200 mM CaCl₂) in acetic buffer, the fifth subspecies elutes at about 95-105 mM CaCl₂).

In some embodiments according to any of the preceding compositions, the second fusion protein is capable of binding to the cell surface of respiratory epithelium.

In some aspects, provided herein is a pharmaceutical composition comprising the composition of any of the preceding embodiments.

In some aspects, provided herein is a method of releasing a composition of any one of the preceding embodiments for human medical use, comprising subjecting the composition to CEX-HPLC, and determining the relative amounts of the first, second, third, fourth, and optionally fifth subspecies separated by the CEX-HPLC.

In some embodiments of the method of releasing, a weight percentage of the first subspecies among the total proteins in the composition being about 50% to about 90% is indicative of the suitability of the composition for human medical use.

In some embodiments according to any of the preceding methods of releasing, a weight percentage of the second subspecies among the total proteins in the composition being about 15% to about 30% is indicative of the suitability of the composition for human medical use.

In some embodiments according to any of the preceding methods of releasing, a weight percentage of the third subspecies among the total proteins in the composition being about 1% to about 15% is indicative of the suitability of the composition for human medical use.

In some embodiments according to any of the preceding methods of releasing, a weight percentage of the fourth subspecies among the total proteins in the composition being about 0.1% to about 4% is indicative of the suitability of the composition for human medical use.

In some embodiments according to any of the preceding methods of releasing, a weight percentage of the fifth subspecies among the total proteins in the composition being about 1% to about 10% is indicative of the suitability of the composition for human medical use.

In some embodiments according to any of the preceding methods of releasing, the CEX-HPLC comprises using non-porous 3 μm polystyrene divinylbenzene bead derivatized with carboxylic acid and elution by a gradient of up to 200 mM CaCl₂) in acetic buffer.

In some aspects, provided herein is a pharmaceutical composition released for human medical use by any of the preceding methods of release.

In some aspects, provided herein is a method of making a pharmaceutical composition comprising the composition of any one of the preceding embodiments, comprising: a) introducing a nucleic acid encoding a protein of SEQ ID NO: 1 into a bacterial host cell; b) expressing the protein encoded by the nucleic acid in the bacterial host cell; c) purifying the protein by chromatography to obtain a purified protein composition; and d) assessing suitability of the purified protein composition for human medical use, wherein the assessing comprises subjecting the purified protein composition to CEX-HPLC and determining the relative amounts of the first, second, third, fourth, and optionally fifth subspecies separated by the CEX-HPLC.

In some embodiments, the method of making further comprises formulating the purified protein composition to obtain a pharmaceutical composition.

In some embodiments according to any of the preceding methods of making, the step of formulating the purified protein composition is carried out after the step of assessing suitability of the purified protein composition.

In some embodiments according to any of the preceding methods of making, the step of formulating the purified protein composition is carried out before the step of assessing suitability of the purified protein composition.

In some aspects, provided herein is a pharmaceutical composition made according to any of the preceding methods of making.

In some aspects according to any of the preceding pharmaceutical compositions, the pharmaceutical composition comprises at least about 70% Trehalose by dry weight.

In some aspects according to any of the preceding pharmaceutical compositions, the pharmaceutical composition comprises at least about 0.2% MgSO₄ by dry weight.

In some aspects according to any of the preceding pharmaceutical compositions, the pharmaceutical composition comprises a) about 95-98% w/w trehalose; b) about 0.2-0.4% w/w MgSO₄; c) about 0.4-0.6% w/w sodium acetate; and d) about 0.1-0.3% w/w acetic acid.

In some aspects, provided herein is a pharmaceutical composition comprising: a) a fusion protein comprising a sialidase domain fused at its C-terminus to a cationic domain; b) about 95-98% w/w trehalose; c) about 0.2-0.4% w/w MgSO₄; d) about 0.4-0.6% w/w sodium acetate; and e) about 0.1-0.3% w/w acetic acid.

In some aspects according to any of the preceding pharmaceutical compositions, the pharmaceutical composition is formulated as a lyophilized formulation.

In some aspects according to any of the preceding pharmaceutical compositions, the composition does not comprise histidine or CaCl₂).

In some aspects according to any of the preceding pharmaceutical compositions, the pharmaceutical composition comprises about 97.5% w/w Trehalose, about 0.3% w/w MgSO₄, about 0.5% w/w sodium acetate, and about 0.2% w/w acetic acid.

In some aspects according to any of the preceding pharmaceutical compositions, the potency of the sialidase in the pharmaceutical composition is about 540-740 U/mg protein.

In some aspects according to any of the preceding pharmaceutical compositions, the pharmaceutical composition upon reconstitution into a liquid formulation has an osmolality of about 270-330 mOsm/kg.

In some aspects according to any of the preceding pharmaceutical compositions, the pharmaceutical composition upon reconstitution into a liquid formulation has a viscosity of about 1.27-1.39 cps.

In some aspects according to any of the preceding pharmaceutical compositions, the pharmaceutical composition upon reconstitution into a liquid formulation has a pH of about 4.5 to about 6.5.

In some aspects according to any of the preceding pharmaceutical compositions, wherein at least about 95% of the proteins in the pharmaceutical composition are monomers.

In some aspects, provided herein is a liquid formulation reconstituted from any one of the preceding pharmaceutical compositions.

In some aspects, provided herein is a vial comprising any one of the preceding pharmaceutical compositions. In some embodiments, the vial is for single use.

In some aspects, provided herein is a nebulizer comprising a liquid formulation, wherein the liquid formulation is reconstituted from any one of the preceding pharmaceutical compositions.

In some aspects, provided herein is a commercial batch comprising any one of the preceding pharmaceutical compositions.

In some aspects, provided herein is a method of treating a disease in an individual comprising administering to the individual an effective amount of any of the preceding pharmaceutical compositions or a liquid formulation reconstituted from said pharmaceutical composition.

### BRIEF DESCRIPTION OF THE DRAWINGS

FIG. 1 shows a representative CEX-HPLC profile of a composition comprising a first, second, third, fourth, and fifth DAS181 subspecies as described herein.

FIG. 2 shows intact mass analysis of the first subspecies.

FIG. 3 shows intact mass analysis of the second subspecies.

FIG. 4 shows intact mass analysis of the third subspecies.

FIG. 5 shows intact mass analysis of the fourth subspecies.

FIG. 6 shows size exclusion chromatography-high performance liquid chromatography (SEC-HPLC) analysis of the fourth subspecies.

FIG. 7 shows sodium dodecyl-sulfate polyacrylamide gel electrophoresis (SDS-PAGE) analysis with Coomassie blue stain detection of the DAS181 variant subspecies by comparison to the Reference Standard DAS181.

FIG. 8A shows cation exchange high performance liquid chromatography (CEX-HPLC) comparison of a liquid reconstituted lyophilized microparticle formulation of the composition at Time 0 and after 2 weeks incubation at 25° C. and 60% relative humidity.

FIG. 8B shows CEX-HPLC of a liquid reconstituted lyophilized cake formulation of the composition at Time 0 and after 2 weeks incubation at 25° C. and 60% relative humidity.

### DETAILED DESCRIPTION

The DAS181 protein comprises a sialidase domain and a charged anchoring domain. During manufacturing of a DAS181 composition, variants of the DAS181 protein, e.g., charge variants, conformational variants, deaminated variants, deamidated variants, truncated variants, oxidized variants, dimers, and/or multimers of DAS181, may be generated. The presence/absence of these variants, as well as the specific amounts of these variants in the composition, depend heavily on the manufacturing process and can significantly impact stability and suitability for clinical use of the DAS181 compositions. The present application provides a novel DAS181 composition generated by a large-scale manufacturing process which is proven to be stable and suitable for clinical use. The novel DAS181 composition comprises DAS181 and specific DAS181 variants, and optionally excipients that are particularly suitable for formulating the DAS181 composition. Also provided are methods of assessing suitability of a DAS181 composition for clinical use based on the presence/absence and/or relative amounts of DAS181 variants.

Thus, in one aspect, provided herein is DAS181 composition comprising a) a first subspecies comprising a first fusion protein comprising a sialidase domain fused via its C-terminus to a cationic domain, wherein the cationic domain comprises amino acids 395-415 of SEQ ID NO: 1; b) a second subspecies comprising a deaminated form of the first fusion protein, wherein the deaminated form of the first fusion protein comprises a deaminated N residue compared to the first fusion protein, and wherein the amino acid position is relative to SEQ ID NO: 1; c) a third subspecies comprising a second fusion protein comprising a sialidase domain fused via its C-terminus to a truncated cationic domain, wherein the truncated cationic domain comprises amino acids 395-406 of SEQ ID NO: 1; and d) a fourth subspecies comprising a dimerized form of the first fusion protein fusion protein comprising a sialidase domain fused at its C-terminus to a cationic domain. In some embodiments, the DAS181 composition further comprises a fifth subspecies comprising a dehydrated form of the first fusion protein.

In some embodiments, provided herein is a method of releasing a DAS181 composition for human medical use, comprising subjecting the composition to CEX-HPLC, and determining the relative amounts of the first, second, third, fourth, and optionally fifth subspecies separated by the CEX-HPLC. Also provided herein are pharmaceutical compositions comprising a mixture of DAS181 subspecies and methods of treating an individual comprising administering same.

In another aspect, provided herein is a lyophilized formulation for a sialidase fusion protein (such as DAS181 or a mixture comprising DAS181 and DAS181 variant subspecies) prepared as a cake, which can be reconstituted and administered via nebulization. In some embodiments, the lyophilized formulation comprises a) about 1.1-1.5% w/w DAS181 or DAS181 and DAS181 variant subspecies; b) about 95-98% w/w trehalose; c) about 0.2-0.4% w/w MgSO₄; d) about 0.4-0.6% w/w sodium acetate; and e) about 0.1-0.3% w/w acetic acid by dry weight. In some embodiments, the formulation comprises about 97.5% w/w Trehalose, about 0.3% w/w MgSO₄, about 0.5% w/w sodium acetate, and about 0.2% w/w acetic acid by dry weight. In another aspect, provided herein is a liquid formulation for a sialidase fusion protein (such as DAS181 or a mixture comprising DAS181 and DAS181 variant subspecies) reconstituted from any of the lyophilized formulations disclosed herein. In some embodiments, the liquid formulation comprises 1-2 mg/mL DAS181 or DAS181 and DAS181 variant subspecies, 10-100 mg/mL trehalose, 0.1-0.5 mg/mL MgSO₄, 0.2-0.7 mg/mL sodium acetate, and 0.1-0.3 mg/mL acetic acid. In some embodiments, the liquid formulation comprises about 1.3 mg/mL DAS181 or DAS181 and DAS181 variant subspecies, about 94 mg/mL trehalose, about 0.3 mg/mL MgSO₄, about 0.5 mg/mL sodium acetate, and about 0.2 mg/mL acetic acid. In some embodiments the liquid formulation comprises 1.3 mg/mL DAS181 or DAS181 and DAS181 variant subspecies, 94 mg/mL trehalose, 0.3 mg/mL MgSO₄, 0.5 mg/mL sodium acetate, and 0.2 mg/mL acetic acid.

### I. Definitions

Terms are used herein as generally used in the art, unless otherwise defined as follows.

As used herein, “DAS181 subspecies” or “subspecies of DAS181” refers to a DAS181 protein (as shown in SEQ ID NO: 1) or a variant or derivative thereof, which has a characteristic elution profile (i.e., under the same peak) by chromatography (e.g., CEX-HPLC) and/or a characteristic set of peaks by intact mass analysis. An individual “DAS181 subspecies” can comprise full-length DAS181 protein as shown in SEQ ID NO: 1, and/or charge variants, conformational variants, deaminated variants, deamidated variants, truncated variants, oxidized variants, dimers, and/or multimers of DAS181. An individual subspecies may comprise one or more than one form or variant of a DAS181 molecule having the amino acid sequence of SEQ ID NO: 1.

As used herein, “DAS181 variant” refers to a portion of a composition of DAS181 that has a different elution profile (i.e., under the same peak) by chromatography (e.g., CEX-HPLC) and/or a characteristic set of peaks by intact mass analysis as compared to a pure, full length, and unmodified DAS181 protein, namely, a monomeric fusion protein comprising a sialidase domain fused via its C-terminus to a cationic domain, wherein the fusion protein has the amino acid sequence of SEQ ID NO: 1. DAS181 variants include, but are not limited to, charge variants, conformational variants, deaminated variants, deamidated variants, truncated variants, oxidized variants, dimers, and/or multimers of DAS181.

As used herein, “treatment” or “treating” is an approach for obtaining beneficial or desired results including clinical results. For purposes of this application, beneficial or desired clinical results include, but are not limited to, one or more of the following: decreasing one more symptoms resulting from the disease, diminishing the extent of the disease, stabilizing the disease (e.g., preventing or delaying the worsening of the disease), preventing or delaying the spread of the disease, preventing or delaying the occurrence or recurrence of the disease, delay or slowing the progression of the disease, ameliorating the disease state, providing a remission (whether partial or total) of the disease, decreasing the dose of one or more other medications required to treat the disease, delaying the progression of the disease, increasing the quality of life, and/or prolonging survival. Also encompassed by “treatment” is a reduction of pathological consequence of the disease. The methods of the present application contemplate any one or more of these aspects of treatment.

The terms “individual,” “subject” and “patient” are used interchangeably herein to describe a mammal, including humans. In some embodiments, the individual is human. In some embodiments, an individual suffers from a cancer. In some embodiments, the individual is in need of treatment.

As is understood in the art, an “effective amount” refers to an amount of a composition sufficient to produce a desired therapeutic outcome (e.g., reducing the severity or duration of, stabilizing the severity of, or eliminating one or more symptoms of cancer). For therapeutic use, beneficial or desired results include, e.g., decreasing one or more symptoms resulting from the disease (biochemical, histologic and/or behavioral), including its complications and intermediate pathological phenotypes presented during development of the disease, increasing the quality of life of those suffering from the disease, decreasing the dose of other medications required to treat the disease, enhancing effect of another medication, delaying the progression of the disease, and/or prolonging survival of patients. In some embodiments, an effective amount of the therapeutic agent may extend survival (including overall survival and progression free survival); result in an objective response (including a complete response or a partial response); relieve to some extent one or more signs or symptoms of the disease or condition; and/or improve the quality of life of the subject.

As used herein the term “wild type” is a term of the art understood by skilled persons and means the typical form of an organism, strain, gene or characteristic as it occurs in nature as distinguished from mutant or variant forms.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

As used herein, “sialidase” refers to a naturally occurring or engineered sialidase that is capable of catalyzing the cleavage of terminal sialic acids from carbohydrates on glycoproteins or glycolipids. As used herein, “sialidase” can refer to a domain of a naturally occurring or non-naturally occurring sialidase that is capable of catalyzing cleavage of terminal sialic acids from carbohydrates on glycoproteins or glycolipids. The term “sialidase” also encompasses fusion proteins comprising a naturally occurring or non-naturally occurring sialidase protein or an enzymatically active fragment or domain thereof and another polypeptide, fragment or domain thereof, e.g., an anchoring domain or a transmembrane domain.

The term “sialidase” as used herein encompasses sialidase catalytic domain proteins. A “sialidase catalytic domain protein” is a protein that comprises the catalytic domain of a sialidase, or an amino acid sequence that is substantially homologous to the catalytic domain of a sialidase, but does not comprise the entire amino acid sequence of the sialidase. The catalytic domain is derived from, wherein the sialidase catalytic domain protein retains substantially the functional activity as the intact sialidase the catalytic domain is derived from. A sialidase catalytic domain protein can comprise amino acid sequences that are not derived from a sialidase. A sialidase catalytic domain protein can comprise amino acid sequences that are derived from or substantially homologous to amino acid sequences of one or more other known proteins, or can comprise one or more amino acids that are not derived from or substantially homologous to amino acid sequences of other known proteins.

As used herein, “expression” refers to the process by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

“Percent (%) amino acid sequence identity” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR), or MUSCLE software. For purposes herein, % amino acid sequence identity values are generated using the sequence comparison computer program MUSCLE (Edgar, R. C., Nucleic Acids Research 32 (5): 1792-1797, 2004; Edgar, R. C., BMC Bioinformatics 5 (1): 113, 2004, each of which are incorporated herein by reference in their entirety for all purposes).

The terms “polypeptide” or “peptide” are used herein to encompass all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to one or more ingredients in a pharmaceutical formulation, other than an active ingredient, which is nontoxic to a subject. A pharmaceutically acceptable carrier includes, but is not limited to, a buffer, excipient, stabilizer, cryoprotectant, tonicity agent, preservative, and combinations thereof. Pharmaceutically acceptable carriers or excipients have preferably met the required standards of toxicological and manufacturing testing and/or are included on the Inactive Ingredient Guide prepared by the U.S. Food and Drug administration or other state/federal government or listed in the U.S. Pharmacopeia or other generally recognized pharmacopeia for use in mammals, and more particularly in humans.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (e.g., a package or container) or kit comprising at least one reagent, e.g., a medicament for treatment of a disease or condition (e.g., cancer), or a probe for specifically detecting a biomarker described herein. In certain embodiments, the manufacture or kit is promoted, distributed, or sold as a unit for performing the methods described herein.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat disease of type X means the method is used to treat disease of types other than X.

The term “about X-Y” used herein has the same meaning as “about X to about Y.”

As used herein and in the appended claims, the singular forms “a,” “an,” or “the” include plural referents unless the context clearly dictates otherwise.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. Compositions

The present application provides compositions comprising multiple subspecies of DAS181, including the first, second, third, fourth, and optional fifth subspecies as described in sections 1.A, 1.B, 1.C, 1.D, and 1.E below.

In some embodiments, the composition comprises: a) a first subspecies comprising a first fusion protein comprising a sialidase domain fused via its C-terminus to a cationic domain, wherein the cationic domain comprises a sequence comprising amino acids 395-415 of SEQ ID NO: 1; b) a second subspecies comprising a deaminated form of the first fusion protein, wherein the deaminated form of the first fusion protein comprises a deaminated N residue compared to the first fusion protein, and wherein the amino acid position is relative to SEQ ID NO: 1; c) a third subspecies comprising a second fusion protein comprising a sialidase domain fused via its C-terminus to a truncated cationic domain, wherein the truncated cationic domain comprises amino acids 395-406 of SEQ ID NO: 1; and d) a fourth subspecies comprising a dimerized form of the first fusion protein. In some embodiments, the composition further comprises a fifth subspecies comprising a dehydrated form of the first fusion protein. In some embodiments, the composition is formulated in a lyophilized cake formulation. In some embodiments, the composition is formulated in a lyophilized microparticle formulation. In some embodiments, the lyophilized formulation is reconstituted in liquid. In some embodiments, the DAS181 composition is a liquid composition (e.g., reconstituted lyophilized composition). In some embodiments, the liquid composition further comprises 10-100 mg/mL trehalose, 0.1-0.5 mg/mL MgSO₄, 0.2-0.7 mg/mL sodium acetate, and 0.1-0.3 mg/mL acetic acid. In some embodiments the liquid formulation comprises about 1.3 mg/mL DAS181 or DAS181 and DAS181 variant subspecies, about 94 mg/mL trehalose, about 0.3 mg/mL MgSO₄, about 0.5 mg/mL sodium acetate, and about 0.2 mg/mL acetic acid. In some embodiments the liquid formulation comprises 1.3 mg/mL DAS181 or DAS181 and DAS181 variant subspecies, 94 mg/mL trehalose, 0.3 mg/mL MgSO₄, 0.5 mg/mL sodium acetate, and 0.2 mg/mL acetic acid.

In some embodiments, when a composition provided herein is subject to cation-exchange chromatography (CEX-HPLC) using non-porous 3 μm polystyrene divinylbenzene bead derivatized with carboxylic acid and elution by a gradient of up to 200 mM CaCl₂ in acetic buffer, the first subspecies elutes at about 80-87 mM CaCl₂), the second subspecies elutes at about 70-75 mM CaCl₂, the third subspecies elutes at about 56-59 mM CaCl₂), and the fourth subspecies elutes at about 180-200 mM CaCl₂). In some embodiments, the composition further comprises a fifth subspecies, wherein when the composition provided herein is subject to cation-exchange chromatography (CEX-HPLC) using non-porous 3 μm polystyrene divinylbenzene bead derivatized with carboxylic acid and elution by a gradient of up to 200 mM CaCl₂) in acetic buffer, the fifth subspecies elutes at about 90-110 mM CaCl₂.

In some embodiments, the CEX-HPLC is performed using the following conditions: Run Time: 78 min; Flow Rate: 1 mL/min. In some embodiments, the CEX-HPLC is from a first buffer (CEX-A) to a second buffer (CEX-B). In some embodiments, CEX-A is 19.5 mM sodium acetate and CEX-B is 19.5 mM sodium acetate with 200 mM CaCl₂. In some embodiments, the CEX-HPLC is performed using the injection percentages of CEX-A and CEX-B at a flow rate of 1 mL/min shown in Table 1 below.

TABLE 1
CEX-HPLC injection percentages of CEX-A and CEX-B

| Time (min) | CEX-A % | CEX-B % |
|---|---|---|
| 1 | 90 | 10 |
| 5 | 73 | 27 |
| 30 | 68 | 32 |
| 31 | 64 | 36 |
| 40 | 64 | 36 |
| 41 | 58 | 42 |
| 50 | 58 | 42 |
| 51 | 50 | 50 |
| 56 | 50 | 50 |
| 57 | 0 | 100 |
| 67 | 0 | 100 |
| 68 | 90 | 10 |
| 78 | 90 | 10 |

In some embodiments, when a composition provided herein is subject to CEX-HPLC using non-porous 3 μm polystyrene divinylbenzene bead derivatized with carboxylic acid and elution according to the conditions shown in Table 1, the first subspecies elutes at 40-45 min, 42-45 min, or about 44 min, the second subspecies elutes at 32-35 min, 33-35 min, or about 34 min, the third subspecies elutes at 12-16 min, 13-15 min, or about 14 min, and the fourth subspecies elutes at 58-62 min, 59-60 min, or about 59.5 min. In some embodiments, the composition comprises a fifth subspecies, and the fifth subspecies elutes at or at about 52-55 min or 53-55 min.

In some embodiments, when a composition provided herein is subject to cation-exchange chromatography (CEX-HPLC) using non-porous 3 μm polystyrene divinylbenzene bead derivatized with carboxylic acid and elution by a gradient of up to 200 mM CaCl₂) in acetic buffer, the first subspecies elutes at about 84 mM CaCl₂), the second subspecies elutes at about 72 mM CaCl₂), the third subspecies elutes at about 57.5 mM CaCl₂), and the fourth subspecies elutes at about 200 mM CaCl₂). In some embodiments, the composition further comprises a fifth subspecies, wherein when the composition provided herein is subject to cation-exchange chromatography (CEX-HPLC) using non-porous 3 μm polystyrene divinylbenzene bead derivatized with carboxylic acid and elution by a gradient of up to 200 mM CaCl₂) in acetic buffer, the fifth subspecies elutes at about 100 mM CaCl₂).

In some embodiments, the weight percentage of the first subspecies among the total proteins in the composition is about 50% to about 90%, the weight percentage of the second subspecies among the total proteins in the composition is about 5% to about 30%, the weight percentage of the third subspecies among the total proteins in the composition is about 1% to about 15%, and the weight percentage of the fourth subspecies among the total proteins in the composition is about 0.1% to about 15% or about 0.1% to about 4%. In some embodiments, the weight percentage of the fifth subspecies among the total proteins in the composition is about 1% to about 15%. In some embodiments, the composition is suitable for human medical use.

In some embodiments, after storage at room temperature, the weight percentage of the first subspecies among the total proteins in the composition is at least about any one of 75%, 80%, 85%, 90%, or more, the weight percentage of the second subspecies among the total proteins is at least about any one of 2, 5%, 10%, 12%, or more, the weight percentage of the third subspecies among the total proteins is at least about any one of 1%, 2%, 3%, 5%, 10%, or more, and the weight percentage of the fourth subspecies among the total proteins is at least about any one of 1%, 2%, 3%, 5%, 10%, or more. In some embodiments, after storage at room temperature, the weight percentage of the first subspecies among the total proteins in the composition is no more than about any one of 90%, 85%, 80%, or 75%, the weight percentage of the second subspecies among the total proteins is no more than about any one of 15%, 14%, 12%, or 10%, the weight percentage of the third subspecies among the total proteins is no more than about any one of 30%, 20%, 15%, 14%, 12%, or 10%, and the weight percentage of the fourth subspecies among the total proteins is no more than about any one of 30%, 20%, 15%, 14%, 12%, or 10%. In some embodiments, after storage at room temperature, the weight percentage of the first subspecies among the total proteins in the composition is between about any one of 80%-85%, 85%-90%, 75%-80%, or 75%-85%, the weight percentage of the second subspecies among the total proteins is any one of about of 5%-10%, 10%-15%, or 15%-20%, the weight percentage of the third subspecies among the total proteins is any one of about 1%-5%, 5%-10%, 10%-15%, 15%-20%, or 20% to 30%, and the weight percentage of the fourth subspecies among the total proteins is any one of about 1%-5%, 5%-10%, 10%-15%, 15%-20%, or 20% to 30%. In some embodiments, after storage at room temperature, the weight percentage of the first species among the total proteins in the composition decrease by no more than about any one of 1%, 2%, 3%, 5%, 10%, 15%, or 20% of that before storage at room temperature. In some embodiments, after storage at room temperature, the weight percentage of the second, third, and/or fourth subspecies increases by no more than about any one of 1%, 2%, 3%, 5%, 10%, 15%, 20%, 30%, or 40% of that before storage at room temperature. In some embodiments, the weight percentage is measured after storage at room temperature for at least about any one of 6 hours, 8 hours, 12 hours, 24 hours, 48 hours, 3 days, 4 days, 5 days, 6 days, 7 days, or more. In some embodiments, the weight percentage is measured after storage at room temperature for no more than about any one of 7 days, 6 days, 5 days, 4 days, 3 days, 48 hours, 24 hours, 12 hours, 8 hours, or 6 hours. In some embodiments, the weight percentage is measured after storage at room temperature for 6-8 hours, 23-25 hours, 47-49 hours, 3-7 days, 6 hours-48 hours, 48 hours-7 days, or 6 hours-7 days. In some embodiments, the composition is suitable for human medical use.

In some embodiments, the first subspecies comprises a variant of a first fusion protein, wherein the first fusion protein comprises a sialidase domain fused via its C-terminus to a cationic domain, wherein the cationic domain comprises amino acids 395-415 of SEQ ID NO: 1, the second subspecies comprises a deaminated form of the first fusion protein, and the fourth subspecies comprises a dimerized and/or methionine oxidized form of the first fusion protein. In some embodiments, the first fusion protein comprises a sialidase domain fused via its C-terminus to a cationic domain, wherein the cationic domain comprises amino acids 395-415 of SEQ ID NO: 1. In some embodiments, the first subspecies further comprises a third fusion protein that lacks the N-terminal M residue comparing to the first fusion protein. In some embodiments, the third fusion protein lacks an amino terminal methionine compared to the first fusion protein.

The third subspecies comprises a second fusion protein comprises a sialidase domain fused via its C-terminus to a truncated cationic domain. “Truncated cationic domain” refers to a cationic domain that is truncated compared to the full-length cationic domain (amino acid residues 396-415) of SEQ ID NO: 1. In some embodiments, the truncated cationic domain is about 1, 2, 3, 4, 5, 6, 7, 8, 9, 10, or 11 amino acids long. In some embodiments, the truncated cationic domain comprises any one of amino acid residues 396, 396-397, 396-398, 396-399, 396-400, 396-401, 396-402, 396-403, 396-404, or 396-495 of SEQ ID NO: 1. In some embodiments, the truncated cationic domain is about 12 amino acids long and comprises amino acids 396-406 of SEQ ID NO: 1. In some embodiments, the truncated cationic domain is about 13 amino acids long and comprises amino acids 396-407 of SEQ ID NO: 1. In some embodiments, the truncated cationic domain is about 14 amino acids long and comprises amino acids 396-408 of SEQ ID NO: 1. In some embodiments, the truncated cationic domain is about 15 amino acids long and comprises amino acids 396-409 of SEQ ID NO: 1. In some embodiments, the truncated cationic domain is about 16 amino acids long and comprises amino acids 396-410 of SEQ ID NO: 1.

In some embodiments, the sialidase domain comprises amino acids 1-394 of SEQ ID NO: 1. In some embodiments, the sialidase domain comprises a sequence having at least 85%, at least 90%, at least 91%, at least 92%, at least 93%, at least 94%, at least 95%, at least 96%, at least 97%, at least 98%, at least 99%, or 100% identity to amino acids 1-394 of SEQ ID NO: 1. In some embodiments, the sialidase domain comprises amino acids 1-394 of SEQ ID NO: 1. In some embodiments, the first fusion protein comprises a sequence having at least 85%, at least 90%, at least 91%, at least 92%, at least 93%, at least 94%, at least 95%, at least 96%, at least 97%, at least 98%, at least 99%, or 100% identity amino acids 1-415 of SEQ ID NO: 1. In some embodiments, the first fusion protein comprises amino acids 1-415 of SEQ ID NO: 1.

In some cases, the amino acids that differ from those in SEQ ID NO: 1 are conservative substitutions. Conservative substitutions may be defined as exchanges within one of the following five groups:
I. Small, aliphatic, nonpolar or slightly polar residues: Ala, Ser, Thr, Pro, Gly
II. Polar, negatively charged residues and their amides: Asp, Asn, Glu, Gln
III. Polar, positively charged residues: His, Arg, Lys
IV. Large, aliphatic nonpolar residues: Met, Leu, Ile, Val, Cys
V. Large aromatic residues: Phe, Try, Trp

Within the foregoing groups, the following substitutions are considered to be “highly conservative”: Asp/Glu, His/Arg/Lys, Phe/Tyr/Trp, and Met/Leu/Ile/Val. Semi-conservative substitutions are defined to be exchanges between two of groups (I)-(IV) above which are limited to supergroup (A), comprising (I), (II), and (III) above, or to supergroup (B), comprising (IV) and (V) above. In addition, where hydrophobic amino acids are specified in the application, they refer to the amino acids Ala, Gly, Pro, Met, Leu, Ile, Val, Cys, Phe, and Trp, whereas hydrophilic amino acids refer to Ser, Thr, Asp, Asn, Glu, Gln, His, Arg, Lys, and Tyr.

### 1. DAS181 Subspecies

### A. First Subspecies

In some embodiments, the first subspecies comprises a first fusion protein comprising a sialidase domain fused via its C-terminus to a cationic domain, wherein the cationic domain comprises amino acids 395-415 of SEQ ID NO: 1. In some embodiments, the sialidase domain comprises amino acids 1-394 of SEQ ID NO: 1. In some embodiments, the first fusion protein comprises amino acids 1-415 of SEQ ID NO: 1.

In some embodiments, at least about 70%, at least about 75%, at least about 80%, at least about 85%, at least about 90%, at least about 95%, at least about 98%, at least about 99%, or 100% by weight of the first subspecies is the sialidase domain fused via its C-terminus to a cationic domain, wherein the cationic domain comprises amino acids 395-415 of SEQ ID NO: 1. In some embodiments, the sialidase domain comprises amino acids 1-394 of SEQ ID NO: 1.

In some embodiments, at least about 50%, at least about 55%, at least about 60%, at least about 65%, at least about 70%, at least about 75%, at least about 80%, or at least about 85% by weight of the first subspecies is the first fusion protein, wherein the first fusion protein comprises amino acids 1-415 of SEQ ID NO: 1.

In some embodiments, between about 50% and about 85%, between about 60% and about 85%, between about 65% and about 85%, or between about 70% and 85% by weight of the first subspecies is the sialidase domain fused via its C-terminus to a cationic domain, wherein the cationic domain comprises amino acids 395-415 of SEQ ID NO: 1. In some embodiments, the sialidase domain comprises amino acids 1-394 of SEQ ID NO: 1.

In some embodiments, the first subspecies further comprises a third fusion protein that lacks the N-terminal M residue comparing to the first fusion protein. In some embodiments, the third fusion protein comprises the amino acid sequence provided in SEQ ID NO: 2. In some embodiments, the ratio of the first fusion protein and the third fusion protein in the first subspecies is about 1:1, about 1.2:1, about 1.3:1, about 1.4:1, about 1.5:1, about 1.6:1, about 1.7:1, about 1.8:1, about 1.9:1, about 2:1, about 2.1:1, about 2.2:1, about 2.3:1, about 2.4:1, about 2.5:1, about 2.6:1, about 2.7:1, about 2.8:1, about 2.9:1, or about 3:1. In some embodiments, the ratio of the first fusion protein and the third fusion protein in the first subspecies is about 2:1.

In some embodiments, the ratio of the first fusion protein and the third fusion protein in the first subspecies is at least about any of 1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, or 3:1. In some embodiments, the ratio of the first fusion protein and the third fusion protein in the first subspecies is at least about 1.5:1.

In some embodiments, the ratio of the first fusion protein and the third fusion protein in the first subspecies is no more than about any of 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, or about 3:1. In some embodiments, the ratio of the first fusion protein and the third fusion protein in the first subspecies is no more than about 2.5:1. In some embodiments, the ratio of the first fusion protein and the third fusion protein in the first subspecies is no more than about 3:1.

In some embodiments, the ratio of the first fusion protein and the third fusion protein is between about 1:1 and about 3:1, between about 1.2:1 and about 2.9:1, between about 1.5:1 and about 2.5:1, or between about 1:1 and about 2:1.

In some embodiments, when a composition provided herein is subject to cation-exchange chromatography (CEX-HPLC) using non-porous 3 μm polystyrene divinylbenzene bead derivatized with carboxylic acid and elution by a gradient of up to 200 mM CaCl₂) in acetic buffer, the first subspecies elutes at about 80-87 mM CaCl₂). In some embodiments, when a composition provided herein is subject to cation-exchange chromatography (CEX-HPLC) using non-porous 3 μm polystyrene divinylbenzene bead derivatized with carboxylic acid and elution by a gradient of up to 200 mM CaCl₂) in acetic buffer, the first subspecies elutes at about 80-85 mM CaCl₂). In some embodiments, when a composition provided herein is subject to cation-exchange chromatography (CEX-HPLC) using non-porous 3 μm polystyrene divinylbenzene bead derivatized with carboxylic acid and elution by a gradient of up to 200 mM CaCl₂) in acetic buffer, the first subspecies elutes at about 82-85 mM CaCl₂. In some embodiments, when a composition provided herein is subject to cation-exchange chromatography (CEX-HPLC) using non-porous 3 μm polystyrene divinylbenzene bead derivatized with carboxylic acid and elution by a gradient of up to 200 mM CaCl₂) in acetic buffer, the first subspecies elutes at about 84 mM CaCl₂).

In some embodiments, the proteins in the first subspecies have at least about any of: 94%, 95%, 96%, 97%, 98%, 99%, 100%, 105%, 110%, 115%, 120%, 125%, 130%, 135%, 140%, or 144% sialidase activity comparing to that of the first fusion protein.

In some embodiments, the proteins in the first subspecies have between any of 80% and 144%, 90% and 144%, 95% and 144%, 80% and 90%, 80% and 95%, 85% and 95%, 95% and 130%, 98% and 130%, or 99% and 130% activity comparing to that of the first fusion protein. In some embodiments, the proteins in the first subspecies have between 90% and 144% activity comparing to that of the first fusion protein.

In some embodiments, the weight percentage of the first subspecies among the total proteins in the composition is any of about 50% to about 90%, about 50% to about 60%, about 60% to about 70%, about 70% to about 80%, about 80% to about 90%, about 50% to about 70%, or about 70% to about 95%. In some embodiments, the weight percentage of the first subspecies among the total proteins in the composition is about 50% to about 90%. In some embodiments, the weight percentage of the first subspecies among the total proteins in the composition is between or between about 70% and 85%.

In some embodiments, the weight percentage of the first subspecies among the total proteins in the composition is at least or at least about any one of 60%, 65%, 70%, 75%, 80%, or 85%. In some embodiments, the weight percentage of the first subspecies among the total proteins in the composition is no more than any one of 90%, 85%, or 80%.

In some embodiments, after 6-8 hours of storage at room temperature, the weight percentage of the first subspecies among the total proteins in the composition is between about 80% and about 85%. In some embodiments, after 6-8 hours of storage at room temperature, the weight percentage of the first subspecies among the total proteins in the composition is between 80% and 85%. In some embodiments, after 23-25 hours of storage at room temperature, the weight percentage of the first subspecies among the total proteins in the composition is between about 80% and about 85%. In some embodiments, after 23-25 hours of storage at room temperature, the weight percentage of the first subspecies among the total proteins in the composition is between 80% and 85%. In some embodiments, after 47-49 hours of storage at room temperature, the weight percentage of the first subspecies among the total proteins in the composition is between about 80% and about 85%. In some embodiments, after 47-49 hours of storage at room temperature, the weight percentage of the first subspecies among the total proteins in the composition is between 80% and 85%. In some embodiments, after about 3 days (e.g., 70-74 hours) of storage at room temperature, the weight percentage of the first subspecies among the total proteins in the composition is between about 80% and about 85%. In some embodiments, about 3 days (e.g., 70-74 hours) of storage at room temperature, the weight percentage of the first subspecies among the total proteins in the composition is between 80% and 85%. In some embodiments, after about 7 days (e.g., 166-170 hours) of storage at room temperature, the weight percentage of the first subspecies among the total proteins in the composition is between about 75% and about 80%. In some embodiments, after about 7 days (e.g., 166-170 hours) of storage at room temperature, the weight percentage of the first subspecies among the total proteins in the composition is between about 78% and about 80%.

In some embodiments, after storage at room temperature, the weight percentage of the first subspecies among the total proteins in the composition is at least about any one of 75%, 80%, 85%, 90%, or more. In some embodiments, after storage at room temperature, the weight percentage of the first subspecies among the total proteins in the composition is no more than about any one of 90%, 85%, 80%, or 75%. In some embodiments, after storage at room temperature, the weight percentage of the first subspecies among the total proteins in the composition is between about any one of 80%-85%, 85%-90%, 75%-80%, or 75%-85%. In some embodiments, after storage at room temperature, the weight percentage of the first species among the total proteins in the composition decrease by no more than about any one of 1%, 2%, 3%, 5%, 10%, 15%, or 20% of that before storage at room temperature. In some embodiments, after storage at room temperature, the weight percentage of the first species among the total proteins in the composition decrease by about any one of 1-20%, 1-5%, 1-10%, 10-20%, or 5-15%. In some embodiments, the weight percentage is measured after storage at room temperature for at least about any one of 6 hours, 8 hours, 12 hours, 24 hours, 48 hours, 3 days, 4 days, 5 days, 6 days, 7 days, or more. In some embodiments, the weight percentage is measured after storage at room temperature for no more than about any one of 7 days, 6 days, 5 days, 4 days, 3 days, 48 hours, 24 hours, 12 hours, 8 hours, or 6 hours. In some embodiments, the weight percentage is measured after storage at room temperature for 6-8 hours, 23-25 hours, 47-49 hours, 3-7 days, 6 hours-48 hours, 48 hours-7 days, or 6 hours-7 days.

In some embodiments, the first subspecies exhibits a first peak having a mass between 44647.42 Da and 44667.42 Da by intact mass analysis. In some embodiments, the first subspecies exhibits a first peak having a mass between 44652.42 Da and 44662.42 Da by intact mass analysis. In some embodiments, the first subspecies exhibits a first peak having a mass between 44653.42 Da and 44661.42 Da by intact mass analysis. In some embodiments, the first subspecies exhibits a first peak having a mass between 44654.42 Da and 44660.42 Da by intact mass analysis. In some embodiments, the first subspecies exhibits a first peak having a mass between 44654.92 Da and 44659.92 Da by intact mass analysis.

In some embodiments, the first subspecies exhibits a second peak having a mass between 44778.61 Da and 44798.61 Da by intact mass analysis. In some embodiments, the first subspecies exhibits a second peak having a mass between 44783.61 Da and 44793.61 Da by intact mass analysis. In some embodiments, the first subspecies exhibits a second peak having a mass between 44784.61 Da and 44792.61 Da by intact mass analysis. In some embodiments, the first subspecies exhibits a second peak having a mass between 44785.61 Da and 44791.61 Da by intact mass analysis. In some embodiments, the first subspecies exhibits a second peak having a mass between 44786.11 Da and 44791.11 Da by intact mass analysis.

In some embodiments, the first subspecies exhibits a third peak having a mass between 44825.42 Da and 44845.42 Da by intact mass analysis. In some embodiments, the first subspecies exhibits a third peak having a mass between 44830.42 Da and 44840.42 Da by intact mass analysis. In some embodiments, the first subspecies exhibits a third peak having a mass between 44831.42 Da and 44839.42 Da by intact mass analysis.

In some embodiments, the third peak represents a gluconate adduct of the first peak. In some embodiments, the third peak has a mass that is greater than the mass of the first peak by between 176 and 180 Da. In some embodiments, the third peak has a mass that is greater than the mass of the first peak by between 177 and 179 Da. In some embodiments, the third peak has a mass that is greater than the mass of the first peak by about 178 Da.

In some embodiments, the first subspecies exhibits a fourth peak having a mass between 44956.63 Da and 44976.63 Da by intact mass analysis. In some embodiments, the first subspecies exhibits a fourth peak having a mass between 44961.63 Da and 44971.63 Da by intact mass analysis. In some embodiments, the first subspecies exhibits a fourth peak having a mass between 44962.63 Da and 44970.63 Da by intact mass analysis.

In some embodiments, the first subspecies exhibits a first peak having a mass between 44647.42 Da and 44667.42 Da by intact mass analysis, a second peak having a mass between 44778.61 Da and 44798.61 Da by intact mass analysis, and a third peak having a mass between 44825.42 Da and 44845.42 Da by intact mass analysis. In some embodiments, the first subspecies further exhibits a fourth peak having a mass between 44956.63 Da and 44976.63 Da by intact mass analysis.

In some embodiments, the first subspecies exhibits a first peak having a mass between 44652.42 Da and 44662.42 Da by intact mass analysis, a second peak having a mass between 44783.61 Da and 44793.61 Da by intact mass analysis, and a third peak having a mass between 44830.42 Da and 44840.42 Da by intact mass analysis. In some embodiments, the first subspecies further exhibits a fourth peak having a mass between 44961.63 Da and 44971.63 Da by intact mass analysis.

In some embodiments, the first subspecies exhibits a first peak having a mass between 44653.42 Da and 44661.42 Da by intact mass analysis, a second peak having a mass between 44784.61 Da and 44792.61 Da by intact mass analysis, and a third peak having a mass between 44831.42 Da and 44839.42 Da by intact mass analysis. In some embodiments, the first subspecies further exhibits a fourth peak having a mass between 44962.63 Da and 44970.63 Da by intact mass analysis.

### B. Second Subspecies

In some embodiments, the second subspecies comprises a deaminated form of the first fusion protein, wherein the first fusion protein is a sialidase domain fused via its C-terminus to a cationic domain, wherein the cationic domain comprises amino acids 395-415 of SEQ ID NO: 1. In some embodiments, the sialidase domain comprises amino acids 1-394 of SEQ ID NO: 1. In some embodiments, the first fusion protein comprises amino acids 1-415 of SEQ ID NO: 1.

In some embodiments, the second subspecies comprises a deaminated and/or deamidated form of the first fusion protein, wherein the first fusion protein is a sialidase domain fused via its C-terminus to a cationic domain, wherein the cationic domain comprises amino acids 395-415 of SEQ ID NO: 1. In some embodiments, the sialidase domain comprises amino acids 1-394 of SEQ ID NO: 1. In some embodiments, the first fusion protein comprises amino acids 1-415 of SEQ ID NO: 1. In some embodiments, deamidation is the major degradation pathway of the first fusion protein comprising the amino acid sequence of SEQ ID NO: 1. In some embodiments, the second subspecies is primarily deamidated and retains full potency.

In some embodiments, the proteins in the second subspecies have at least or at least about any one of 80%, 85%, 90%, 95%, or 100% sialidase activity comparing to that of the first fusion protein. In some embodiments, the proteins in the second subspecies have at least or at least about 80% sialidase activity comparing to that of the first fusion protein. In some embodiments, the proteins in the second subspecies have at least or at least about 85% sialidase activity comparing to that of the first fusion protein. In some embodiments, the proteins in the second subspecies have at least or at least about 86% sialidase activity comparing to that of the first fusion protein.

In some embodiments, the weight percentage of the second subspecies among the total proteins in the composition is about 5% to about 30%. In some embodiments, the weight percentage of the second subspecies among the total proteins in the composition is any one of about 5% to about 10%, about 5% to about 20%, about 10% to about 20%, about 10% to about 15%, or about 5% to about 15%. In some embodiments, the weight percentage of the second subspecies among the total proteins in the composition is about 10% to about 30%. In some embodiments, the weight percentage of the second subspecies among the total proteins in the composition is at least or at least about any one of 5%, 10%, 15%, 20%, or 25%. In some embodiments, the weight percentage of the second subspecies among the total proteins in the composition is no more than or no more than about any one of 30%, 25%, or 20%.

In some embodiments, the weight percentage of the second subspecies among the total proteins in the composition is at least or at least about any one of 1%, 2%, 3%, 4%, 5%, 6%, 7%, 8%, 9%, 10%, 12%, 13%, or 14%. In some embodiments, the weight percentage of the second subspecies among the total proteins in the composition is at least or at least about 1%. In some embodiments, the weight percentage of the second subspecies among the total proteins in the composition is at least or at least about 5%. In some embodiments, the weight percentage of the second subspecies among the total proteins in the composition is at least or at least about 10%.

In some embodiments, when the composition is subject to cation-exchange chromatography (CEX-HPLC) using non-porous 3 μm polystyrene divinylbenzene bead derivatized with carboxylic acid and elution by a gradient of up to 200 mM CaCl₂) in acetic buffer, the second subspecies elutes at about 70-75 mM CaCl₂). In some embodiments, when the composition is subject to cation-exchange chromatography (CEX-HPLC) using non-porous 3 μm polystyrene divinylbenzene bead derivatized with carboxylic acid and elution by a gradient of up to 200 mM CaCl₂) in acetic buffer, the second subspecies elutes at about 71-74 mM CaCl₂). In some embodiments, when the composition is subject to cation-exchange chromatography (CEX-HPLC) using non-porous 3 μm polystyrene divinylbenzene bead derivatized with carboxylic acid and elution by a gradient of up to 200 mM CaCl₂) in acetic buffer, the second subspecies elutes at about 72 mM CaCl₂).

In some embodiments, after 6-8 hours of storage at room temperature, the weight percentage of the second subspecies among the total proteins in the composition is between about 5% and about 15% or between about 10% and about 15%. In some embodiments, after 23-25 hours of storage at room temperature, the weight percentage of the second subspecies among the total proteins in the composition is between about 5% and about 15% or between about 10% and about 15%. In some embodiments, after 47-49 hours of storage at room temperature, the weight percentage of the second subspecies among the total proteins in the composition is between about 5% and about 15% or between about 10% and about 15%. In some embodiments, after about 3 days (e.g., 70-74 hours) of storage at room temperature, the weight percentage of the second subspecies among the total proteins in the composition is between about 5% and about 15% or between about 10% and about 15%. In some embodiments, after about 7 days (e.g., 166-170 hours) of storage at room temperature, the weight percentage of the second subspecies among the total proteins in the composition is between about 5% and about 15% or between about 10% and about 15%.

In some embodiments, after storage at room temperature, the weight percentage of the second subspecies among the total proteins in the composition is at least about any one of 2, 5%, 10%, 12% or more. In some embodiments, after storage at room temperature, the weight percentage of the second subspecies among the total proteins in the composition is no more than about any one of 15%, 14%, 12%, or 10%. In some embodiments, after storage at room temperature, the weight percentage of the second subspecies among the total proteins in the composition is between about any one of 5%-10%, 10%-15%, or 15%-20%. In some embodiments, after storage at room temperature, the weight percentage of the second subspecies among the total proteins in the composition increase by no more than about any one of 1%, 2%, 3%, 5%, 10%, 15%, or 20% of that before storage at room temperature. In some embodiments, the weight percentage is measured after storage at room temperature for at least about any one of 6 hours, 8 hours, 12 hours, 24 hours, 48 hours, 3 days, 4 days, 5 days, 6 days, 7 days, or more. In some embodiments, the weight percentage is measured after storage at room temperature for no more than about any one of 7 days, 6 days, 5 days, 4 days, 3 days, 48 hours, 24 hours, 12 hours, 8 hours, or 6 hours. In some embodiments, the weight percentage is measured after storage at room temperature for 6-8 hours, 23-25 hours, 47-49 hours, 3-7 days, 6 hours-48 hours, 48 hours-7 days, or 6 hours-7 days.

In some embodiments, the second subspecies exhibits a first peak having a mass between 44647.42 Da and 44667.42 Da by intact mass analysis. In some embodiments, the second subspecies exhibits a first peak having a mass between 44652.42 Da and 44662.42 Da by intact mass analysis. In some embodiments, the second subspecies exhibits a first peak having a mass between 44653.42 Da and 44661.42 Da by intact mass analysis. In some embodiments, the second subspecies exhibits a peak having a mass between 44654.42 Da and 44660.42 Da by intact mass analysis. In some embodiments, the second subspecies exhibits a peak having a mass between 44654.92 Da and 44659.92 Da by intact mass analysis.

In some embodiments, the second subspecies exhibits a second peak having a mass between 44778.61 Da and 44798.61 Da by intact mass analysis. In some embodiments, the second subspecies exhibits a second peak having a mass between 44783.61 Da and 44793.61 Da by intact mass analysis. In some embodiments, the second subspecies exhibits a second peak having a mass between 44784.61 Da and 44792.61 Da by intact mass analysis. In some embodiments, the second subspecies exhibits a second peak having a mass between 44785.61 Da and 44791.61 Da by intact mass analysis. In some embodiments, the second subspecies exhibits a second peak having a mass between 44786.11 Da and 44791.11 Da by intact mass analysis.

In some embodiments, the second subspecies exhibits a third peak having a mass between 44825.42 Da and 44845.42 Da by intact mass analysis. In some embodiments, the second subspecies exhibits a third peak having a mass between 44830.42 Da and 44840.42 Da by intact mass analysis. In some embodiments, the second subspecies exhibits a third peak having a mass between 44831.42 Da and 44839.42 Da by intact mass analysis. In some embodiments, the third peak represents a gluconate adduct of the first peak. In some embodiments, the third peak has a mass that is greater than the mass of the first peak by between 176 and 180 Da. In some embodiments, the third peak has a mass that is greater than the mass of the first peak by between 177 and 179 Da. In some embodiments, the third peak has a mass that is greater than the mass of the first peak by about 178 Da.

In some embodiments, the second subspecies exhibits a fourth peak having a mass between 44956.63 Da and 44976.63 Da by intact mass analysis. In some embodiments, the second subspecies exhibits a fourth peak having a mass between 44961.63 Da and 44971.63 Da by intact mass analysis. In some embodiments, the second subspecies exhibits a fourth peak having a mass between 44962.63 Da and 44970.63 Da by intact mass analysis.

In some embodiments, the second subspecies exhibits a first peak having a mass between 44647.42 Da and 44667.42 Da by intact mass analysis, a second peak having a mass between 44778.61 Da and 44798.61 Da by intact mass analysis, and a third peak having a mass between 44825.42 Da and 44845.42 Da by intact mass analysis. In some embodiments, the second subspecies further exhibits a fourth peak having a mass between 44956.63 Da and 44976.63 Da by intact mass analysis.

In some embodiments, the second subspecies exhibits a first peak having a mass between 44652.42 Da and 44662.42 Da by intact mass analysis, a second peak having a mass between 44783.61 Da and 44793.61 Da by intact mass analysis, and a third peak having a mass between 44830.42 Da and 44840.42 Da by intact mass analysis. In some embodiments, the second subspecies further exhibits a fourth peak having a mass between 44961.63 Da and 44971.63 Da by intact mass analysis.

In some embodiments, the second subspecies exhibits a first peak having a mass between 44653.42 Da and 44661.42 Da by intact mass analysis, a second peak having a mass between 44784.61 Da and 44792.61 Da by intact mass analysis, and a third peak having a mass between 44831.42 Da and 44839.42 Da by intact mass analysis. In some embodiments, the second subspecies further exhibits a fourth peak having a mass between 44962.63 Da and 44970.63 Da by intact mass analysis.

### C. Third Subspecies

The third subspecies comprises a second fusion protein comprising a sialidase domain fused via its C-terminus to a truncated cationic domain. “Truncated cationic domain” refers to a cationic domain that is truncated compared to the full-length cationic domain (amino acid residues 396-415) of SEQ ID NO: 1. In some embodiments, the truncated cationic domain is about 1, 2, 3, 4, 5, 6, 7, 8, 9, 10, or 11 amino acids long. In some embodiments, the truncated cationic domain comprises any one of amino acid residues 396, 396-397, 396-398, 396-399, 396-400, 396-401, 396-402, 396-403, 396-404, or 396-495 of SEQ ID NO: 1. In some embodiments, the truncated cationic domain is about 12 amino acids long and comprises amino acids 396-406 of SEQ ID NO: 1. In some embodiments, the truncated cationic domain is about 13 amino acids long and comprises amino acids 396-407 of SEQ ID NO: 1. In some embodiments, the truncated cationic domain is about 14 amino acids long and comprises amino acids 396-408 of SEQ ID NO: 1. In some embodiments, the truncated cationic domain is about 15 amino acids long and comprises amino acids 396-409 of SEQ ID NO: 1. In some embodiments, the truncated cationic domain is about 16 amino acids long and comprises amino acids 396-410 of SEQ ID NO: 1. In some embodiments, the sialidase domain comprises amino acids 1-394 of SEQ ID NO: 1. In some embodiments, the second fusion protein comprises amino acids 1-406 of SEQ ID NO: 1. In some embodiments, the third subspecies further comprises oxidized or deaminated forms of the second fusion protein.

In some embodiments, the second fusion protein comprising the truncated cationic domain is capable of binding to the cell surface of respiratory epithelium. In some embodiments, the second fusion protein has at least or at least about any one of 80%, 85%, 86%, 87%, 88%, 89%, 90%, 91%, 92%, 93%, 94%, 95%, 96%, 97%, 98%, 99%, or 100% binding to the cell surface of respiratory epithelium comparing to binding of the first fusion protein to the cell surface of respiratory epithelium. In some embodiments, the second fusion protein has at least or at least about 85% binding to the cell surface of respiratory epithelium comparing to binding of the first fusion protein to the cell surface of respiratory epithelium.

In some embodiments, the second fusion protein has between 80% and 90%, between 85% and 90%, between 80% and 100%, between 80% and 110%, between 100% and 120%, between 120% and 150%, or between 95% and 100% binding to the cell surface of respiratory epithelium comparing to binding of the first fusion protein to the cell surface of respiratory epithelium.

In some embodiments, the proteins in the third subspecies have at least or at least about any one of 80%, 85%, 86%, 87%, 88%, 89%, 90%, 91%, 92%, 93%, 94%, 95%, 96%, 97%, 98%, 99%, or 100% sialidase activity comparing to that of the first fusion protein. In some embodiments, the proteins in the third subspecies have at least or at least about 80% sialidase activity comparing to that of the first fusion protein. In some embodiments, the proteins in the third subspecies have at least or at least about 85% sialidase activity comparing to that of the first fusion protein.

In some embodiments, the second fusion protein has between 80% and 90%, between 85% and 90%, between 80% and 100%, between 80% and 110%, between 100% and 120%, between 120% and 150%, or between 95% and 100% sialidase activity comparing to that of the first fusion protein.

In some embodiments, the weight percentage of the third subspecies among the total proteins in the composition is about 1% to about 15%, about 1% to about 10%, about 5% to about 10%, about 2% to about 15%, about 3% to about 15%, about 4% to about 15%, or about 5% to about 15%. In some embodiments, the weight percentage of the third subspecies among the total proteins in the composition is about 1% to about 15%. In some embodiments, the weight percentage of the third subspecies among the total proteins in the composition is about 1% to about 14%.

In some embodiments, the weight percentage of the third subspecies among the total proteins in the composition is at least or at least about any one of 1%, 1.2%, a 1.3%, 1.5%, 2%, or 3%. In some embodiments, the weight percentage of the third subspecies among the total proteins in the composition is at least or at least about 1%. In some embodiments, the weight percentage of the third subspecies among the total proteins in the composition is at least or at least about 3%.

In some embodiments, the weight percentage of the third subspecies among the total proteins in the composition is no more than or no more than about any one of 30%, 20%, 15%, or 10%. In some embodiments, the weight percentage of the third subspecies among the total proteins in the composition is no more than about 14%.

In some embodiments, when the composition is subject to cation-exchange chromatography (CEX-HPLC) using non-porous 3 μm polystyrene divinylbenzene bead derivatized with carboxylic acid and elution by a gradient of up to 200 mM CaCl₂) in acetic buffer, the third subspecies elutes at about 56-59 mM CaCl₂). In some embodiments, when the composition is subject to cation-exchange chromatography (CEX-HPLC) using non-porous 3 μm polystyrene divinylbenzene bead derivatized with carboxylic acid and elution by a gradient of up to 200 mM CaCl₂) in acetic buffer, the third subspecies elutes at about 57-58 mM CaCl₂. In some embodiments, when the composition is subject to cation-exchange chromatography (CEX-HPLC) using non-porous 3 μm polystyrene divinylbenzene bead derivatized with carboxylic acid and elution by a gradient of up to 200 mM CaCl₂) in acetic buffer, the third subspecies elutes at about 57.5 mM CaCl₂).

In some embodiments, after 6-8 hours of storage at room temperature, the weight percentage of the third subspecies among the total proteins in the composition is between about 1% and about 5% or between about 4% and about 5%. In some embodiments, after 23-25 hours of storage at room temperature, the weight percentage of the third subspecies among the total proteins in the composition is between about 1% and about 5% or between about 4% and about 5%. In some embodiments, after 47-49 hours of storage at room temperature, the weight percentage of the third subspecies among the total proteins in the composition is between about 1% and about 5% or between about 4% and about 5%. In some embodiments, after about 3 days (e.g., 70-74 hours) of storage at room temperature, the weight percentage of the third subspecies among the total proteins in the composition is between about 1% and about 5% or between about 4% and about 5%. In some embodiments, after about 7 days (e.g., 166-170 hours) of storage at room temperature, the weight percentage of the third subspecies among the total proteins in the composition is between about 1% and about 5% or between about 4% and about 5%.

In some embodiments, after storage at room temperature, the weight percentage of the third subspecies among the total proteins in the composition is at least about any one of 1%, 2%, 3%, 5%, 10%, or more. In some embodiments, after storage at room temperature, the weight percentage of the third subspecies among the total proteins in the composition is no more than about any one of 30%, 20%, 15%, 14%, 12%, or 10%. In some embodiments, after storage at room temperature, the weight percentage of the third subspecies among the total proteins in the composition is between about any one of 1%-5%, 5%-10%, 10%-15%, 15%-20%, or 20% to 30%. In some embodiments, after storage at room temperature, the weight percentage of the third subspecies among the total proteins in the composition increase by no more than about any one of 1%, 2%, 3%, 5%, 10%, 15%, or 20% of that before storage at room temperature. In some embodiments, after storage at room temperature, the weight percentage of the third subspecies among the total proteins in the composition decreases by no more than about any one of 1%, 2%, 3%, 5%, 10%, 15%, or 20% of that before storage at room temperature. In some embodiments, the weight percentage is measured after storage at room temperature for at least about any one of 6 hours, 8 hours, 12 hours, 24 hours, 48 hours, 3 days, 4 days, 5 days, 6 days, 7 days, or more. In some embodiments, the weight percentage is measured after storage at room temperature for no more than about any one of 7 days, 6 days, 5 days, 4 days, 3 days, 48 hours, 24 hours, 12 hours, 8 hours, or 6 hours. In some embodiments, the weight percentage is measured after storage at room temperature for 6-8 hours, 23-25 hours, 47-49 hours, 3-7 days, 6 hours-48 hours, 48 hours-7 days, or 6 hours-7 days.

In some embodiments, the third subspecies exhibits a first peak having a mass between 43539.50 Da and 43559.50 Da by intact mass analysis. In some embodiments, the third subspecies exhibits a first peak having a mass between 43544.50 Da and 43554.50 Da by intact mass analysis. In some embodiments, the third subspecies exhibits a first peak having a mass between 43545.50 Da and 43553.50 Da by intact mass analysis. In some embodiments, the third subspecies exhibits a first peak having a mass between 43546.50 Da and 43552.50 Da by intact mass analysis. In some embodiments, the third subspecies exhibits a first peak having a mass between 43547.50 Da and 43551.50 Da by intact mass analysis. In some embodiments, the third subspecies exhibits a first peak having a mass between 43548.50 Da and 43550.50 Da by intact mass analysis.

In some embodiments, the third subspecies exhibits a second peak having a mass between 43671.20 Da and 43691.20 Da by intact mass analysis. In some embodiments, the third subspecies exhibits a second peak having a mass between 43676.20 Da and 43686.20 Da by intact mass analysis. In some embodiments, the third subspecies exhibits a second peak having a mass between 43677.20 Da and 43685.20 Da by intact mass analysis. In some embodiments, the third subspecies exhibits a second peak having a mass between 43678.20 Da and 43684.20 Da by intact mass analysis. In some embodiments, the third subspecies exhibits a second peak having a mass between 43679.20 Da and 43683.20 Da by intact mass analysis. In some embodiments, the third subspecies exhibits a second peak having a mass between 43680.20 Da and 43682.20 Da by intact mass analysis.

In some embodiments, the third subspecies exhibits a third peak having a mass between 43717.50 Da and 43737.50 Da by intact mass analysis. In some embodiments, the third subspecies exhibits a third peak having a mass between 43722.50 Da and 43732.50 Da by intact mass analysis. In some embodiments, the third subspecies exhibits a third peak having a mass between 43723.50 Da and 43731.50 Da by intact mass analysis. In some embodiments, the third subspecies exhibits a third peak having a mass between 43724.50 Da and 43730.50 Da by intact mass analysis. In some embodiments, the third subspecies exhibits a third peak having a mass between 43725.50 Da and 43729.50 Da by intact mass analysis. In some embodiments, the third subspecies exhibits a third peak having a mass between 43726.50 Da and 43728.50 Da by intact mass analysis.

In some embodiments, the third subspecies exhibits a first peak having a mass between 43539.50 Da and 43559.50 Da by intact mass analysis, a second peak having a mass between 43671.20 Da and 43691.20 Da by intact mass analysis, and a third peak having a mass between 43717.50 Da and 43737.50 Da by intact mass analysis.

In some embodiments, the third subspecies exhibits a first peak having a mass between 43544.50 Da and 43554.50 Da by intact mass analysis, a second peak having a mass between 43676.20 Da and 43686.20 Da by intact mass analysis, and a third peak having a mass between 43722.50 Da and 43732.50 Da by intact mass analysis.

In some embodiments, the third subspecies exhibits a first peak having a mass between 43545.50 Da and 43553.50 Da by intact mass analysis, a second peak having a mass between 43677.20 Da and 43685.20 Da by intact mass analysis, and a third peak having a mass between 43723.50 Da and 43731.50 Da by intact mass analysis.

In some embodiments, the third subspecies exhibits a first peak having a mass between 43546.50 Da and 43552.50 Da by intact mass analysis, a second peak having a mass between 43678.20 Da and 43684.20 Da by intact mass analysis, and a third peak having a mass between 43724.50 Da and 43730.50 Da by intact mass analysis.

### D. Fourth Subspecies

The fourth subspecies comprises a dimerized form of the first fusion protein. In some embodiments, the first fusion protein comprises wherein the first fusion protein comprises a sialidase domain fused via its C-terminus to a cationic domain, wherein the cationic domain comprises amino acids 395-415 of SEQ ID NO: 1. In some embodiments, the sialidase domain comprises amino acids 1-394 of SEQ ID NO: 1. In some embodiments, the first fusion protein comprises amino acids 1-415 of SEQ ID NO: 1.

In some embodiments, the fourth subspecies comprises a methionine oxidized form of the first fusion protein. In some embodiments, the first fusion protein comprises a sialidase domain fused via its C-terminus to a cationic domain, wherein the cationic domain comprises amino acids 395-415 of SEQ ID NO: 1. In some embodiments, the sialidase domain comprises amino acids 1-394 of SEQ ID NO: 1. In some embodiments, the first fusion protein comprises amino acids 1-415 of SEQ ID NO: 1.

In some embodiments, the proteins in the fourth subspecies have at least or at least about any one of 40%, 45%, 50%, 60%, 65%, 70%, 75%, 80%, 85%, 90%, 95%, or 100% sialidase activity comparing to that of the first fusion protein. In some embodiments, the proteins in the fourth subspecies have at least or at least about 40% sialidase activity comparing to that of the first fusion protein. In some embodiments, the proteins in the fourth subspecies have at least or at least about 45% sialidase activity comparing to that of the first fusion protein. In some embodiments, the proteins in the fourth subspecies have at least or at least about 90% sialidase activity comparing to that of the first fusion protein.

In some embodiments, the fourth subspecies comprises any of about 40% dimer and about 60% monomers, about 35% dimers and about 65% monomers, about 30% dimer and about 70% monomers, about 25% dimer and about 75% monomers, about 20% dimer and about 80% monomers, about 15% dimer and about 85% monomers, or about 10% dimer and about 90% monomers. In some embodiments, the fourth subspecies comprises about 35% dimers and about 65% monomers.

In some embodiments, the fourth subspecies comprises a methionine oxidized form of the first fusion protein. Methionine oxidized forms of the fourth subspecies of the first fusion protein include, e.g., any number of oxidized methionines such as one or all, or combinations thereof. In some embodiments, at least or at least about any one of 20%, 25%, 30%, 35%, 40%, 45%, 50%, or 55% of the fourth subspecies by weight is a methionine oxidized form of the first fusion protein.

In some embodiments, between 20% and 55%, between 20% and 45%, between 20% and 30%, between 30% and 40%, between 40% and 45%, between 40% and 55%, between 30% and 55%, or between 35% and 55% of the fourth subspecies by weight is a methionine oxidized form of the first fusion protein.

In some embodiments, no more than or no more than about 60%, 55%, 50%, 45%, 40%, 35%, 30%, 25%, or 20% of the fourth subspecies by weight is a methionine oxidized form of the first fusion protein.

In some embodiments, the weight percentage of the fourth subspecies among the total proteins in the composition is any one of about 0.1% to about 15%, about 0.1% to about 10%, about 0.1% to about 5%, about 0.1% to about 4%, about 0.2% to about 4%, about 0.3% to about 4%, about 0.4% to about 4%, about 0.5% to about 4%, about 0.6% to about 4%, about 0.7% to about 4%, about 0.8% to about 4%, about 0.9% to about 4%, about 1% to about 4%, about 2% to about 4%, about 0.2% to about 3%, about 0.3% to about 3%, about 0.4% to about 3%, about 0.5% to about 3%, about 0.6% to about 3%, about 0.7% to about 3%, about 0.8% to about 3%, about 0.9% to about 3%, about 1% to about 3%, 2% to about 3% about 0.1% to about 2%, about 0.2% to about 2%, about 0.3% to about 2%, about 0.4% to about 2%, about 0.5% to about 2%, about 0.6% to about 2%, about 0.7% to about 2%, about 0.8% to about 2%, about 0.9% to about 2%, or about 1% to about 2%. In some embodiments, the weight percentage of the fourth subspecies among the total proteins in the composition is about 0.1% to about 4%.

In some embodiments, the weight percentage of the fourth subspecies among the total proteins in the composition is at least or at least about any one of 0.1%, 0.2%, 0.3%, 0.4%, 0.5%, 0.6%, 0.7%, 0.8%, 0.9%, 1%, 1.1%, 1.2%, or 1.3%. In some embodiments, the weight percentage of the fourth subspecies among the total proteins in the composition is at least or at least about 0.5%. In some embodiments, the weight percentage of the fourth subspecies among the total proteins in the composition is at least or at least about 1%.

In some embodiments, the weight percentage of the fourth subspecies among the total proteins in the composition is no more than or no more than about any one of 20%, 15%, 10%, 5%, 4%, or 3%. In some embodiments, the weight percentage of the fourth subspecies among the total proteins in the composition is no more than about 10%. In some embodiments, the weight percentage of the fourth subspecies among the total proteins in the composition is no more than about 4%.

In some embodiments, when the composition is subject to cation-exchange chromatography (CEX-HPLC) using non-porous 3 μm polystyrene divinylbenzene bead derivatized with carboxylic acid and elution by a gradient of up to 200 mM CaCl₂) in acetic buffer, the fourth subspecies elutes at about 180-200 mM CaCl₂. In some embodiments, when the composition is subject to cation-exchange chromatography (CEX-HPLC) using non-porous 3 μm polystyrene divinylbenzene bead derivatized with carboxylic acid and elution by a gradient of up to 200 mM CaCl₂) in acetic buffer, the fourth subspecies elutes at about 185-200 mM CaCl₂. In some embodiments, when the composition is subject to cation-exchange chromatography (CEX-HPLC) using non-porous 3 μm polystyrene divinylbenzene bead derivatized with carboxylic acid and elution by a gradient of up to 200 mM CaCl₂) in acetic buffer, the fourth subspecies elutes at about 190-200 mM CaCl₂). In some embodiments, when the composition is subject to cation-exchange chromatography (CEX-HPLC) using non-porous 3 μm polystyrene divinylbenzene bead derivatized with carboxylic acid and elution by a gradient of up to 200 mM CaCl₂) in acetic buffer, the fourth subspecies elutes at about 195-200 mM CaCl₂. In some embodiments, when the composition is subject to cation-exchange chromatography (CEX-HPLC) using non-porous 3 μm polystyrene divinylbenzene bead derivatized with carboxylic acid and elution by a gradient of up to 200 mM CaCl₂) in acetic buffer, the fourth subspecies elutes at about 200 mM CaCl₂).

In some embodiments, after 6-8 hours of storage at room temperature, the weight percentage of the fourth subspecies among the total proteins in the composition is between about 1% and about 5% or between about 1% and about 3%. In some embodiments, after 23-25 hours of storage at room temperature, the weight percentage of the fourth subspecies among the total proteins in the composition is between about between about 1% and about 5% or between about 1% and about 3%. In some embodiments, after 47-49 hours of storage at room temperature, the weight percentage of the fourth subspecies among the total proteins in the composition is between about 1% and about 5% or between about 1% and about 3%. In some embodiments, after about 3 days (e.g., 70-74 hours) of storage at room temperature, the weight percentage of the fourth subspecies among the total proteins in the composition is between about 1% and about 5% or between about 1% and about 3%. In some embodiments, after about 7 days (e.g., 166-170 hours) of storage at room temperature, the weight percentage of the fourth subspecies among the total proteins in the composition is between about 1% and about 5% or between about 1% and about 3%.

In some embodiments, after storage at room temperature, the weight percentage of the fourth subspecies among the total proteins in the composition is at least about any one of 1%, 2%, 3%, 5%, 10%, or more. In some embodiments, after storage at room temperature, the weight percentage of the fourth subspecies among the total proteins in the composition is no more than about any one of 30%, 20%, 15%, 14%, 12%, or 10%. In some embodiments, after storage at room temperature, the weight percentage of the fourth subspecies among the total proteins in the composition is between about any one of 1%-5%, 5%-10%, 10%-15%, 15%-20%, or 20% to 30%. In some embodiments, after storage at room temperature, the weight percentage of the fourth subspecies among the total proteins in the composition increase by no more than about any one of 1%, 2%, 3%, 5%, 10%, 15%, or 20% of that before storage at room temperature. In some embodiments, after storage at room temperature, the weight percentage of the fourth subspecies among the total proteins in the composition decreases by no more than about any one of 1%, 2%, 3%, 5%, 10%, 15%, or 20% of that before storage at room temperature. In some embodiments, the weight percentage is measured after storage at room temperature for at least about any one of 6 hours, 8 hours, 12 hours, 24 hours, 48 hours, 3 days, 4 days, 5 days, 6 days, 7 days, or more. In some embodiments, the weight percentage is measured after storage at room temperature for no more than about any one of 7 days, 6 days, 5 days, 4 days, 3 days, 48 hours, 24 hours, 12 hours, 8 hours, or 6 hours. In some embodiments, the weight percentage is measured after storage at room temperature for 6-8 hours, 23-25 hours, 47-49 hours, 3-7 days, 6 hours-48 hours, 48 hours-7 days, or 6 hours-7 days.

In some embodiments, the fourth subspecies exhibits a first peak having a mass between 44647.42 Da and 44667.42 Da by intact mass analysis. In some embodiments, the fourth subspecies exhibits a first peak having a mass between 44652.42 Da and 44662.42 Da by intact mass analysis. In some embodiments, the fourth subspecies exhibits a first peak having a mass between 44653.42 Da and 44661.42 Da by intact mass analysis. In some embodiments, the fourth subspecies exhibits a peak having a mass between 44654.42 Da and 44660.42 Da by intact mass analysis. In some embodiments, the fourth subspecies exhibits a peak having a mass between 44654.92 Da and 44659.92 Da by intact mass analysis.

In some embodiments, the fourth subspecies exhibits a second peak having a mass between 44663.42 Da and 44683.42 Da by intact mass analysis. In some embodiments, the fourth subspecies exhibits a second peak having a mass between 44668.42 Da and 44678.42 Da by intact mass analysis. In some embodiments, the fourth subspecies exhibits a second peak having a mass between 44669.42 Da and 44677.42 Da by intact mass analysis. In some embodiments, the fourth subspecies exhibits a second peak having a mass between 44670.42 Da and 44676.42 Da by intact mass analysis. In some embodiments, the fourth subspecies exhibits a second peak having a mass between 44670.92 Da and 44675.92 Da by intact mass analysis. In some embodiments, the second peak of the fourth species comprises a methionine oxidized variant of the first peak of the fourth species. In some embodiments, the second peak has a mass that is greater than the mass of the first peak by between 15 and 17 Da. In some embodiments, the second peak has a mass that is greater than the mass of the first peak by about 16 Da.

In some embodiments, the fourth subspecies exhibits a third peak having a mass between 44825.42 Da and 44845.42 Da by intact mass analysis. In some embodiments, the fourth subspecies exhibits a third peak having a mass between 44830.42 Da and 44840.42 Da by intact mass analysis. In some embodiments, the fourth subspecies exhibits a third peak having a mass between 44831.42 Da and 44839.42 Da by intact mass analysis. In some embodiments, the third peak comprises a gluconate adduct of the first peak. In some embodiments, the third peak has a mass that is greater than the mass of the first peak by between 176 and 180 Da. In some embodiments, the third peak has a mass that is greater than the mass of the first peak by between 177 and 179 Da. In some embodiments, the third peak has a mass that is greater than the mass of the first peak by about 178 Da.

### E. Fifth Subspecies

In some embodiments, a composition provided herein can optionally comprise a fifth subspecies. The fifth subspecies comprises a dehydrated form of the first fusion protein. In some embodiments, the dehydrated form of the first fusion protein comprises loss, e.g., loss of association, of at least one or more water molecules, as compared to a fully hydrated form of the fifth subspecies. In some embodiments, the first fusion protein comprises a sialidase domain fused via its C-terminus to a cationic domain, wherein the cationic domain comprises amino acids 395-415 of SEQ ID NO: 1. In some embodiments, the sialidase domain comprises amino acids 1-394 of SEQ ID NO: 1. In some embodiments, the first fusion protein comprises amino acids 1-415 of SEQ ID NO: 1.

In some embodiments, the weight percentage of the fifth subspecies among the total proteins in the composition is about 1% to about 15%, about 1% to about 3%, about 2% to about 3%, about 5% to about 15%, about 10% to 15%, about 5% to about 10%, or about 1% to about 10%. In some embodiments, the weight percentage of the fifth subspecies among the total proteins in the composition is about 1% to about 10%. In some embodiments, the weight percentage of the fifth subspecies among the total proteins in the composition is about 1% to about 9%. In some embodiments, the weight percentage of the fifth subspecies among the total proteins in the composition is about 2% to about 9%.

In some embodiments, the weight percentage of the fifth subspecies among the total proteins in the composition is at least or at least about any one of 0.1%, 0.2%, 0.5%, 1%, 2%, 3%, 4%, 5%, or 10%. In some embodiments, the weight percentage of the fifth subspecies among the total proteins in the composition is at least or at least about 0.5%. In some embodiments, the weight percentage of the fifth subspecies among the total proteins in the composition is at least or at least about 1%.

In some embodiments, the weight percentage of the fifth subspecies among the total proteins in the composition is no more than or no more than about any one of 15%, 10%, 9%, 8%, 7%, 6%, 5%, 4%, or 3%. In some embodiments, the weight percentage of the fifth subspecies among the total proteins in the composition is no more than about 10%.

In some embodiments, after 6-8 hours of storage at room temperature, the weight percentage of the fifth subspecies among the total proteins in the composition is between about 1% and about 5% or between about 1% and about 3%. In some embodiments, after 23-25 hours of storage at room temperature, the weight percentage of the fifth subspecies among the total proteins in the composition is between about between about 1% and about 5% or between about 1% and about 3%. In some embodiments, after 47-49 hours of storage at room temperature, the weight percentage of the fifth subspecies among the total proteins in the composition is between about 1% and about 5% or between about 1% and about 3%. In some embodiments, after about 3 days (e.g., 70-74 hours) of storage at room temperature, the weight percentage of the fifth subspecies among the total proteins in the composition is between about 1% and about 5% or between about 1% and about 3%. In some embodiments, after about 7 days (e.g., 166-170 hours) of storage at room temperature, the weight percentage of the fifth subspecies among the total proteins in the composition is between about 1% and about 5% or between about 1% and about 3%.

In some embodiments, after storage at room temperature, the weight percentage of the fifth subspecies among the total proteins in the composition is at least about any one of 1%, 2%, 3%, 5%, 10%, or more. In some embodiments, after storage at room temperature, the weight percentage of the fifth subspecies among the total proteins in the composition is no more than about any one of 30%, 20%, 15%, 14%, 12%, or 10%. In some embodiments, after storage at room temperature, the weight percentage of the fifth subspecies among the total proteins in the composition is between about any one of 1%-5%, 5%-10%, 10%-15%, 15%-20%, or 20% to 30%. In some embodiments, after storage at room temperature, the weight percentage of the fifth subspecies among the total proteins in the composition increase by no more than about any one of 1%, 2%, 3%, 5%, 10%, 15%, or 20% of that before storage at room temperature. In some embodiments, after storage at room temperature, the weight percentage of the fifth subspecies among the total proteins in the composition decreases by no more than about any one of 1%, 2%, 3%, 5%, 10%, 15%, or 20% of that before storage at room temperature. In some embodiments, the weight percentage is measured after storage at room temperature for at least about any one of 6 hours, 8 hours, 12 hours, 24 hours, 48 hours, 3 days, 4 days, 5 days, 6 days, 7 days, or more. In some embodiments, the weight percentage is measured after storage at room temperature for no more than about any one of 7 days, 6 days, 5 days, 4 days, 3 days, 48 hours, 24 hours, 12 hours, 8 hours, or 6 hours. In some embodiments, the weight percentage is measured after storage at room temperature for 6-8 hours, 23-25 hours, 47-49 hours, 3-7 days, 6 hours-48 hours, 48 hours-7 days, or 6 hours-7 days.

In some embodiments, when the composition is subject to cation-exchange chromatography (CEX-HPLC) using non-porous 3 μm polystyrene divinylbenzene bead derivatized with carboxylic acid and elution by a gradient of up to 200 mM CaCl₂) in acetic buffer, the fifth subspecies elutes at about 90-110 mM CaCl₂). In some embodiments, when the composition is subject to cation-exchange chromatography (CEX-HPLC) using non-porous 3 μm polystyrene divinylbenzene bead derivatized with carboxylic acid and elution by a gradient of up to 200 mM CaCl₂) in acetic buffer, the fifth subspecies elutes at about 95-105 mM CaCl₂). In some embodiments, when the composition is subject to cation-exchange chromatography (CEX-HPLC) using non-porous 3 μm polystyrene divinylbenzene bead derivatized with carboxylic acid and elution by a gradient of up to 200 mM CaCl₂) in acetic buffer, the fifth subspecies elutes at about 100 mM CaCl₂.

In some embodiments, a weight percentage of the fifth subspecies among the total proteins in the composition being about 0.5% to about 10%, about 1% to about 3%, about 2% to about 3%, about 5% to about 15%, about 10% to 15%, about 5% to about 10%, or about 1% to about 10% is indicative of the suitability of the composition for human medical use. In some embodiments, a weight percentage of the fifth subspecies among the total proteins in the composition being about 1% to about 9% is indicative of the suitability of the composition for human medical use. In some embodiments, a weight percentage of the fifth subspecies among the total proteins in the composition being about 1% to about 8% is indicative of the suitability of the composition for human medical use.

### 2. Formulations

In some aspects, the compositions provided herein are provided in lyophilized microparticle or lyophilized cake formulations, or in a liquid formulation reconstituted from a lyophilized formulation. In some embodiments, the liquid formulation is provided in a nebulizer, such as a vibrating mesh nebulizer. In some embodiments, a composition provided herein is provided in a dry powder formulation. In some embodiments, a composition provided herein is provided in a lyophilized formulation (e.g., a lyophilized microparticle powder or lyophilized cake) for liquid reconstitution. In some embodiments, there is provided a liquid composition that is reconstituted from a lyophilized formulation.

### A. Lyophilized Cake Formulations for Liquid Reconstitution

In some aspects, provided herein is a lyophilized formulation (e.g., a lyophilized cake) of a fusion protein comprising a sialidase domain fused at its C-terminus to a cationic domain. In some aspects, provided herein is a lyophilized cake formulation of DAS181 or a composition comprising at least four subspecies of DAS181 as described in Section II.1 above. In some embodiments, provided herein is a pharmaceutical formulation comprising: a) a fusion protein comprising a sialidase domain fused at its C-terminus to a cationic domain; b) about 95-98% w/w trehalose; c) about 0.2-0.4% w/w MgSO₄; d) about 0.4-0.6% w/w sodium acetate; and e) about 0.1-0.3% w/w acetic acid. Unless otherwise stated, the weight percentages provided in Section II.2.B describe the dry weight percentages in the lyophilized cake.

In some embodiments, the formulation comprises between any of about 0.5% w/w and about 10% w/w, about 0.5% w/w and about 9% w/w, about 0.5% w/w and about 8% w/w, about 0.5% w/w and about 7% w/w, about 0.5% w/w and about 6% w/w, about 0.5% w/w and about 5% w/w, about 0.5% w/w and about 4% w/w, 0.5% w/w and about 3% w/w, or about 0.5% w/w and about 2% w/w active agent.

In some embodiments, the formulation comprises between any of about 1% w/w and about 10% w/w, about 1% w/w and about 9% w/w, about 1% w/w and about 8% w/w, about 1% w/w and about 7% w/w, about 1% w/w and about 6% w/w, about 1% w/w and about 5% w/w active agent, about 1% w/w and about 4% w/w active agent, about 1% w/w and about 3% w/w active agent, about 1% w/w and about 2% w/w, or about 1.2% w/w and 1.4% w/w active agent. In some embodiments, the formulation comprises about any one of 1.1%, 1.2%, 1.3%, 1.4%, 1.5%, 1.6%, 1.7%, 1.8%, 1.9%, or 2% w/w active agent. In some embodiments, the formulation comprises between 1.2% w/w and 1.4% w/w active agent. In some embodiments, the formulation comprises about 1.3% w/w active agent. In some embodiments, the formulation comprises 1.3% w/w active agent.

In some embodiments, the formulation comprises at least about any one of 1.1%, 1.2%, 1.3%, 1.4%, 1.5%, 1.6%, 1.7%, 1.8%, 1.9%, or 2% w/w active agent. In some embodiments, the formulation comprises at least 1.1% w/w active agent.

In some embodiments, the formulation comprises no more than or no more than about any one of 1.3%, 1.4%, 1.5%, 1.6%, 1.7%, 1.8%, 1.9%, or 2% w/w active agent. In some embodiments, the formulation comprises no more than 3% w/w active agent, no more than 4% w/w active agent, no more than 5% w/w active agent, or no more than 10% w/w active agent.

In some embodiments, the active agent is DAS181, whose sequence is set forth in SEQ ID NO: 1 or SEQ ID NO: 2 (no amino terminal methionine). In some embodiments, the active agent is A) a polypeptide comprising (or consisting of or consisting essentially of) the amino acid sequence of SEQ ID NO: 1; B) a polypeptide comprising (or consisting of or consisting essentially of) the amino acid sequence of SEQ ID NO: 2; or C) a mixture of a polypeptides comprising (or consisting of or consisting essentially of) SEQ ID NO: 1 and polypeptides comprising (or consisting of or consisting essentially of) SEQ ID NO: 2.

In some embodiments, the active agent is a mixture of DAS181 and variants thereof comprising at least four subspecies as described in Section II.1 above. In some embodiments, the active agent comprises or consists of a mixture of DAS181 that can be separated by CEX-HPLC is provided, wherein the formulation comprises: a) a first subspecies comprising a first fusion protein comprising a sialidase domain fused via its C-terminus to a cationic domain, wherein the cationic domain comprises amino acids 395-415 of SEQ ID NO: 1; b) a second subspecies comprising a deaminated form of the first fusion protein, wherein the deaminated form of the first fusion protein comprises a deaminated N residue compared to the first fusion protein, and wherein the amino acid position is relative to SEQ ID NO: 1; c) a third subspecies comprising a second fusion protein comprising a sialidase domain fused via its C-terminus to a truncated cationic domain, wherein the truncated cationic domain comprises amino acids 395-406 of SEQ ID NO: 1; and d) a fourth subspecies comprising a dimerized form of the first fusion protein. In some embodiments, the mixture further comprises the fifth subspecies of DAS181 described in Section II.1 above.

In some embodiments, the formulation comprises between any of about 70% w/w and about 99% w/w, about 75% w/w and about 99% w/w, about 80% w/w and about 99% w/w trehalose, about 85% w/w and about 99% w/w trehalose, about 90% w/w and about 99% w/w trehalose, about 95% w/w and about 99% w/w, 95% w/w and 99% w/w trehalose, about 95% w/w and about 98%, 95% w/w and 98% w/w, 96% w/w and 99% w/w, or 97% w/w and 99% w/w trehalose. In some embodiments, the formulation comprises about 97.5% w/w trehalose or 97.5% w/w trehalose.

In some embodiments, the formulation comprises at least or at least about any one of 70%, 75%, 80%, 85%, 90%, 95%, 96%, or 97% w/w trehalose. In some embodiments, the formulation comprises at least or at least about 95% w/w trehalose. In some embodiments, the formulation comprises at least or at least about 97% w/w trehalose.

In some embodiments, the formulation comprises no more than or no more than about any one of 99%, 98%, or 97.5% w/w trehalose.

In some embodiments, the formulation comprises between any one of about 0.15% w/w and about 0.45% w/w, about 0.2% w/w and about 0.4% w/w, or about 0.25% w/w and about 0.35% w/w MgSO₄. In some embodiments, the formulation comprises between 0.25% w/w and 0.35% w/w MgSO₄. In some embodiments, the formulation comprises about 0.3% w/w MgSO₄ or 0.3% w/w MgSO₄.

In some embodiments, the formulation comprises at least or at least about any one of 0.1%, 0.15%, 0.2%, 0.25%, or 0.3% w/w MgSO₄. In some embodiments, the formulation comprises at least or at least about 0.2% w/w MgSO₄

In some embodiments, the formulation comprises, no more than or no more than about any one of 3%, 2%, 1%, 0.7%, 0.5%, 0.4%, or 0.3% w/w MgSO₄. In some embodiments, the formulation comprises no more than about 0.5% w/w MgSO₄

In some embodiments, the formulation comprises between any one of about 0.3% w/w and about 0.7% w/w, 0.4% w/w and about 0.6% w/w, or about 0.45% w/w and about 0.55% w/w sodium acetate. In some embodiments, the formulation comprises between 0.45% w/w and 0.55% w/w sodium acetate. In some embodiments, the formulation comprises about 0.5% w/w or 0.5% w/w sodium acetate.

In some embodiments, the formulation comprises at least or at least about 0.1% w/w sodium acetate, at least or at least about 0.2% w/w sodium acetate, at least or at least about 0.25% w/w sodium acetate, at least or at least about 0.3% w/w sodium acetate, at least or at least about 0.4% w/w sodium acetate, or at least or at least about 0.5% w/w sodium acetate. In some embodiments, the formulation comprises at least or at least about 0.3% w/w sodium acetate.

In some embodiments, the formulation comprises no more than about 2% w/w sodium acetate, no more than about 1% w/w sodium acetate, no more than about 0.8% w/w sodium acetate, no more than about 0.7% w/w sodium acetate, no more than about 0.6% w/w sodium acetate, or no more than about 0.5% w/w sodium acetate. In some embodiments, the formulation comprises no more than about 0.7% w/w sodium acetate.

In some embodiments, the formulation has a pH of between about 4.5 and about 6.5, between about 5.4 and about 6.4, or between about 5 and about 6. In some embodiments, the formulation has a pH of between 5 and 6. In some embodiments, the formulation has a pH of 5.3 or about 5.3.

In some embodiments, the formulation has a pH of at least or at least about any one of 4, 4.5, 4.6, 4.7, 4.8, 4.9, or 5.0. In some embodiments, the formulation has a pH of at least or at least about 4.7.

In some embodiments, the formulation has a pH of no more than or no more than about any one of 7.0, 6.5, 6.3, 6.0, 5.9, 5.8, or 5.7. In some embodiments, the formulation has a pH of no more than about 6.5. In some embodiments, the formulation has a pH or no more than about 6.3.

In some embodiments, the formulation comprises a buffering agent. In some embodiments, the buffering agent has a pH of between about 4.5 and about 6.5, between about 5.4 and about 6.4, or between about 5 and about 6. In some embodiments, the buffering agent is acetic acid. In some embodiments, the formulation comprises between about 0.1% w/w and about 0.3% w/w acetic acid or between about 0.15% w/w and about 0.25% w/w acetic acid. In some embodiments, the formulation comprises 0.2% or about 0.2% w/w acetic acid. In some embodiments, the formulation comprises at least or at least about any one of 0.05%, 0.1%, 0.15%, or 0.2% w/w acetic acid. In some embodiments, the formulation comprises at least or at least about 0.1% w/w acetic acid. In some embodiments, the formulation comprises no more than or no more than about any one of 3%, 2%, 1%, 0.5%, 0.4%, 0.3%, or 0.2% w/w acetic acid. In some embodiments, the formulation comprises no more than about 0.5% w/w acetic acid.

In some embodiments, the formulation does not comprise histidine. In some embodiments, the formulation does not comprise CaCl₂).

In some embodiments, provided herein is a reconstituted liquid formulation produced by reconstituting the lyophilized cake formulation in water. In some embodiments, the reconstituted liquid formulation comprises between about 70 mg/mL and about 100 mg/mL, between about 75 mg/mL and about 100 mg/mL, between about 80 mg/mL and about 100 mg/mL, between about 85 mg/mL and about 100 mg/mL, or between about 90 mg/mL and about 100 mg/mL trehalose. In some embodiments, the reconstituted liquid formulation comprises between about 91 mg/mL and about 100 mg/mL, between about 92 mg/mL and about 100 mg/mL trehalose, between about 93 mg/mL and about 100 mg/mL, between about 90 mg/mL and about 99 mg/mL, between about 90 mg/mL and about 98 mg/mL, between about 90 mg/mL and about 97 mg/mL, between about 90 mg/mL and about 96 mg/mL, between about 90 mg/mL and about 95 mg/mL, or between about 92 mg/mL and about 96 mg/mL trehalose. In some embodiments, the reconstituted liquid formulation comprises between 92 mg/mL and 96 mg/mL trehalose. In some embodiments, the reconstituted liquid formulation comprises between 93 mg/mL and 95 mg/mL trehalose. In some embodiments, the reconstituted liquid formulation comprises about 94 mg/mL trehalose. In some embodiments, the reconstituted liquid formulation comprises 94 mg/mL trehalose.

In some embodiments, the reconstituted liquid formulation comprises at least or at least about any one of 70, 80, 90, 91, 92, 93, or 94 mg/mL trehalose. In some embodiments, the reconstituted liquid formulation comprises at least or at least about 90 mg/mL trehalose. In some embodiments, the reconstituted liquid formulation comprises no more than or no more than about any one of 100, 99, 98, 97, 96, or 95 mg/mL trehalose. In some embodiments, the reconstituted liquid formulation comprises no more than about 96 mg/mL trehalose.

In some embodiments, the reconstituted liquid formulation comprises between about 0.1 mg/mL and about 0.3 mg/mL acetic acid. In some embodiments, the reconstituted liquid formulation comprises between 0.1 mg/mL and 0.3 mg/mL acetic acid. In some embodiments, the reconstituted liquid formulation comprises between about 0.15 mg/mL and about 0.25 mg/mL acetic acid. In some embodiments, the reconstituted liquid formulation comprises between 0.15 mg/mL and 0.25 mg/mL acetic acid. In some embodiments, the reconstituted liquid formulation comprises about 0.2 mg/mL acetic acid. In some embodiments, the reconstituted liquid formulation comprises 0.2 mg/mL acetic acid.

In some embodiments, the reconstituted liquid formulation comprises at least or at least about 0.05 mg/mL acetic acid. In some embodiments, the reconstituted liquid formulation comprises at least or at least about 0.1 mg/mL acetic acid. In some embodiments, the reconstituted liquid formulation comprises at least or at least about 0.15 mg/mL acetic acid. In some embodiments, the reconstituted liquid formulation comprises at least or at least about 0.2 mg/mL acetic acid.

In some embodiments, the reconstituted liquid formulation comprises no more than about 1 mg/mL acetic acid, no more than about 0.8 mg/mL acetic acid, no more than about 0.7 mg/mL acetic acid, no more than about 0.6 mg/mL acetic acid, no more than about 0.5 mg/mL acetic acid, no more than about 0.4 mg/mL acetic acid, or no more than about 0.3 mg/mL acetic acid. In some embodiments, the reconstituted liquid formulation comprises no more than about 0.4 mg/mL acetic acid.

In some embodiments, the reconstituted liquid formulation comprises between about 0.4 mg/mL and about 0.6 mg/mL, between about 0.45 mg/mL and about 0.55 mg/mL, or between 0.45 mg/mL and 0.55 mg/mL sodium acetate. In some embodiments, the reconstituted liquid formulation comprises 0.5 mg/mL or about 0.5 mg/mL sodium acetate.

In some embodiments, the reconstituted liquid formulation comprises at least or at least about any one of 0.1, 0.2, 0.3, 0.4, or 0.5 mg/mL sodium acetate. In some embodiments, the reconstituted liquid formulation comprises at least or at least about 0.3 mg/mL sodium acetate. In some embodiments, the reconstituted liquid formulation comprises no more than or no more than about any one of 1, 0.8, 0.9, 0.7, or 0.6 mg/mL sodium acetate. In some embodiments, the reconstituted liquid formulation comprises no more than about 0.7 mg/mL sodium acetate.

In some embodiments, the reconstituted liquid formulation comprises between about 0.2 mg/mL and about 0.5 mg/mL MgSO₄ or between about 0.2 mg/mL and about 0.4 mg/mL MgSO₄. In some embodiments, the reconstituted liquid formulation comprises about 0.3 mg/mL MgSO₄. In some embodiments, the reconstituted liquid formulation comprises 0.3 mg/mL MgSO₄. In some embodiments, the formulation does not comprise histidine or CaCl₂.

In some embodiments, the reconstituted liquid formulation comprises at least or at least about any one of 0.05, 0.1, 0.15, 0.2, or 0.3 mg/mL MgSO₄. In some embodiments, the reconstituted liquid formulation comprises at least or at least about 0.1 mg/mL MgSO₄.

In some embodiments, the reconstituted liquid formulation comprises no more than or no more than about any one of 1, 0.9, 0.8, 0.7, 0.6, 0.5, or 0.4 mg/mL MgSO₄. In some embodiments, the reconstituted liquid formulation comprises no more than about 0.5 mg/mL MgSO₄.

In some embodiments, the reconstituted liquid formulation comprises between or between about any one of 0.5 mg/mL and 5 mg/mL, 0.5 mg/mL and 4 mg/mL, 0.5 mg/mL and 3 mg/mL, 0.5 mg/mL and 2 mg/mL, 1 mg/mL and 1.5, 1.1 mg/mL and 2 mg/mL, or 1.2 mg/mL and 2 mg/mL active agent. In some embodiments, the reconstituted liquid formulation comprises at least or at least about any one of 0.9, 1, 1.1, 1.2, or 1.3 mg/mL active agent. In some embodiments, the reconstituted liquid formulation comprises at least or at least about 1 mg/mL active agent. In some embodiments, the reconstituted liquid formulation comprises no more than or no more than about any one of 3, 2.5, 2, 1.9, 1.8, 1.7, 1.6, 1.5, or 1.4 mg/mL active agent. In some embodiments, the reconstituted liquid formulation comprises no more than about 1.8 mg/mL active agent.

In some embodiments, the reconstituted liquid formulation comprises 1-2 mg/mL DAS181 and DAS181 variant subspecies, 10-100 mg/mL trehalose, 0.1-0.5 mg/mL MgSO₄, 0.2-0.7 mg/mL sodium acetate, and 0.1-0.3 mg/mL acetic acid. In some embodiments, the reconstituted liquid formulation comprises about 1.3 mg/mL DAS181 and DAS181variant subspecies, about 94 mg/mL trehalose, about 0.3 mg/mL MgSO₄, about 0.5 mg/mL sodium acetate, and about 0.2 mg/mL acetic acid. In some embodiments, the reconstituted liquid formulation comprises 1.3 mg/mL DAS181 and DAS181 variant subspecies, 94 mg/mL trehalose, 0.3 mg/mL MgSO₄, 0.5 mg/mL sodium acetate, and 0.2 mg/mL acetic acid.

In some embodiments, provided herein is a pharmaceutical formulation comprising: a) a fusion protein comprising a sialidase domain fused at its C-terminus to a cationic domain; b) about 95-98% w/w trehalose; c) about 0.2-0.4% w/w MgSO₄; d) about 0.4-0.6% w/w sodium acetate; and e) about 0.1-0.3% w/w acetic acid by dry weight. In some embodiments, the pharmaceutical formulation comprises about 97.5% w/w Trehalose, about 0.3% w/w MgSO₄, about 0.5% w/w sodium acetate, and about 0.2% w/w acetic acid by dry weight. In some embodiments, the pharmaceutical formulation is a liquid formulation reconstituted from a lyophilized formulation. In some embodiments, the liquid formulation comprises 1-2 mg/mL DAS181 and DAS181 variant subspecies, 10-100 mg/mL trehalose, 0.1-0.5 mg/mL MgSO₄, 0.2-0.7 mg/mL sodium acetate, and 0.1-0.3 mg/mL acetic acid. In some embodiments the liquid formulation comprises about 1.3 mg/mL DAS181 and DAS181 variant subspecies, about 94 mg/mL trehalose, about 0.3 mg/mL MgSO₄, about 0.5 mg/mL sodium acetate, and about 0.2 mg/mL acetic acid. In some embodiments the liquid formulation comprises 1.3 mg/mL DAS181 and DAS181 variant subspecies, 94 mg/mL trehalose, 0.3 mg/mL MgSO₄, 0.5 mg/mL sodium acetate, and 0.2 mg/mL acetic acid. In some embodiments, the fusion protein is DAS181, whose sequence is set forth in SEQ ID NO: 1 (amino terminal methionine present) or SEQ ID NO: 2 (no amino terminal methionine). In some embodiments, the fusion protein is A) a polypeptide comprising (or consisting of or consisting essentially of) the amino acid sequence of SEQ ID NO: 1; B) a polypeptide comprising (or consisting of or consisting essentially of) the amino acid sequence of SEQ ID NO: 2; or C) a mixture of a polypeptides comprising (or consisting of or consisting essentially of) SEQ ID NO: 1 and polypeptides comprising (or consisting of or consisting essentially of) SEQ ID NO: 2.

In some embodiments, provided herein is a pharmaceutical formulation comprising: i) a mixture of DAS181 subspecies that can be separated by CEX-HPLC, comprising a) a first subspecies comprising a first fusion protein comprising a sialidase domain fused via its C-terminus to a cationic domain, wherein the cationic domain comprises amino acids 395-415 of SEQ ID NO: 1; b) a second subspecies comprising a deaminated form of the first fusion protein, wherein the deaminated form of the first fusion protein comprises a deaminated N residue compared to the first fusion protein, and wherein the amino acid position is relative to SEQ ID NO: 1; c) a third subspecies comprising a second fusion protein comprising a sialidase domain fused via its C-terminus to a truncated cationic domain, wherein the truncated cationic domain comprises amino acids 395-406 of SEQ ID NO: 1; and d) a fourth subspecies comprising a dimerized form of the first fusion protein fusion protein comprising a sialidase domain fused at its C-terminus to a cationic domain; ii) about 95-98% w/w trehalose; iii) about 0.2-0.4% w/w MgSO₄; iv) about 0.4-0.6% w/w sodium acetate; and v) about 0.1-0.3% w/w acetic acid by dry weight. In some embodiments, the pharmaceutical formulation comprises about 97.5% w/w Trehalose, about 0.3% w/w MgSO₄, about 0.5% w/w sodium acetate, and about 0.2% w/w acetic acid by dry weight. In some embodiments, the pharmaceutical formulation is a liquid formulation reconstituted from a lyophilized formulation. In some embodiments, the liquid formulation comprises 1-2 mg/mL DAS181 and DAS181 variant subspecies, 10-100 mg/mL trehalose, 0.1-0.5 mg/mL MgSO₄, 0.2-0.7 mg/mL sodium acetate, and 0.1-0.3 mg/mL acetic acid. In some embodiments the liquid formulation comprises about 1.3 mg/mL DAS181 protein (including subspecies), about 94 mg/mL trehalose, about 0.3 mg/mL MgSO₄, about 0.5 mg/mL sodium acetate, and about 0.2 mg/mL acetic acid. In some embodiments the liquid formulation comprises 1.3 mg/mL DAS181 and DAS181 variant subspecies, 94 mg/mL trehalose, 0.3 mg/mL MgSO₄, 0.5 mg/mL sodium acetate, and 0.2 mg/mL acetic acid. In some embodiments, the liquid formulation is provided in a nebulizer, such as a vibrating mesh nebulizer.

In some aspects, provided herein is a nebulizer comprising a liquid formulation comprising DAS181 or a composition comprising multiple subspecies of DAS181 or variants thereof. In some embodiments, the nebulizer is a vibrating mesh nebulizer. In some embodiments, the liquid formulation comprises 1-2 mg/mL DAS181 and DAS181 variant subspecies, 10-100 mg/mL trehalose, 0.1-0.5 mg/mL MgSO₄, 0.2-0.7 mg/mL sodium acetate, and 0.1-0.3 mg/mL acetic acid. In some embodiments the liquid formulation comprises about 1.3 mg/mL DAS181 protein (including subspecies), about 94 mg/mL trehalose, about 0.3 mg/mL MgSO₄, about 0.5 mg/mL sodium acetate, and about 0.2 mg/mL acetic acid. In some embodiments the liquid formulation comprises 1.3 mg/mL DAS181 and DAS181 variant subspecies, 94 mg/mL trehalose, 0.3 mg/mL MgSO₄, 0.5 mg/mL sodium acetate, and 0.2 mg/mL acetic acid. In some embodiments, the DAS181 and DAS181 variant subspecies comprises a mixture of DAS181 subspecies that can be separated by CEX-HPLC, comprising a) a first subspecies comprising a first fusion protein comprising a sialidase domain fused via its C-terminus to a cationic domain, wherein the cationic domain comprises amino acids 395-415 of SEQ ID NO: 1; b) a second subspecies comprising a deaminated form of the first fusion protein, wherein the deaminated form of the first fusion protein comprises a deaminated N residue compared to the first fusion protein, and wherein the amino acid position is relative to SEQ ID NO: 1; c) a third subspecies comprising a second fusion protein comprising a sialidase domain fused via its C-terminus to a truncated cationic domain, wherein the truncated cationic domain comprises amino acids 395-406 of SEQ ID NO: 1; and d) a fourth subspecies comprising a dimerized form of the first fusion protein fusion protein comprising a sialidase domain fused at its C-terminus to a cationic domain.

### B. Lyophilized Microparticle Formulations

In some embodiments, a formulation comprising at least four subspecies of DAS181 that can be separated by CEX-HPLC is provided, wherein the formulation comprises: a) a first subspecies comprising a first fusion protein comprising a sialidase domain fused via its C-terminus to a cationic domain, wherein the cationic domain comprises amino acids 395-415 of SEQ ID NO: 1; b) a second subspecies comprising a deaminated form of the first fusion protein, wherein the deaminated form of the first fusion protein comprises a deaminated N residue compared to the first fusion protein, and wherein the amino acid position is relative to SEQ ID NO: 1; c) a third subspecies comprising a second fusion protein comprising a sialidase domain fused via its C-terminus to a truncated cationic domain, wherein the truncated cationic domain comprises amino acids 395-406 of SEQ ID NO: 1; and d) a fourth subspecies comprising a dimerized form of the first fusion protein, and the formulation is formulated in a microparticle formulation. Suitable lyophilized microparticle formulations are described in U.S. Pat. No. 9,700,602, the content of which is herein incorporated by reference in its entirety.

In some embodiments, a formulation comprising at least four subspecies of DAS181 is provided in the following microparticle formulation (also referred herein as “Formulation II—not anhydrous”:
a) DAS181: 64.5-64.7% (w/w %) total protein comprising at least four subspecies of DAS181 as described in Section II. 1 above
b) Histidine free base: 4.3-4.6% (w/w %)
c) Histidine HCl: 5.8-6.3% (w/w %)
d) Trehalose: 9.0-9.7% (w/w %)
e) Magnesium sulfate: 4.6-5.9% (w/w %)
g) Water: 10.0% (w/w %, depending on humidity of storage conditions)

In some embodiments, the formulation can also include small amounts of sodium acetate (less than 1% w/w, less than 0.5% w/w, less than 0.1% w/w, less than 0.05% w/w, e.g., 0.03% w/w); small amounts of calcium chloride (less than 1% w/w, less than 0.5% w/w, less than 0.3% w/w, e.g., 0.3% w/w); and small amounts of acetic acid (less than 1% w/w, less than 0.5% w/w, less than 0.1% w/w, less than 0.05% w/w, e.g., 0.02% w/w). Small amounts of residual isopropanol can sometimes be present (less than 1% w/w, less than 0.5% w/w, less than 0.1% w/w, less than 0.05% w/w, less than 0.01% w/w).

In some embodiments, a formulation comprising at least four subspecies of DAS181 is provided in the following dry microparticle formulation (also referred herein as “Formulation II-anhydrous”:
a) DAS181: 71.7-71.9% (w/w %) of total protein comprising at least four subspecies of DAS181 as described in Section II. 1 above
b) Histidine free base: 4.8-5.1% (w/w %)
c) Histidine HCl: 6.5-7.0% (w/w %)
d) Trehalose: 10.7-10.1% (w/w %)
e) Magnesium sulfate: 5.1-6.5% (w/w %)

In some embodiments, the formulation can also include small amounts of sodium acetate (less than 1% w/w, less than 0.5% w/w, less than 0.1% w/w, less than 0.05% w/w, e.g., 0.03% w/w); small amounts of calcium chloride (less than 1% w/w, less than 0.5% w/w, less than 0.3% w/w, e.g., 0.3% w/w); and small amounts of acetic acid (less than 1% w/w, less than 0.5% w/w, less than 0.1% w/w, less than 0.05% w/w, e.g., 0.02% w/w). Small amounts of residual isopropanol can sometimes be present (less than 1% w/w, less than 0.5% w/w, less than 0.1% w/w, less than 0.05% w/w, less than 0.01% w/w). In some embodiments the formulation is free of citrate.

In one embodiment, a method of preparing Formulation II described above includes the following steps:
(a) An Excipient Solution (pH 6) containing histidine, trehalose, magnesium sulfate and calcium chloride is prepared by combining stock solutions is prepared and sterile filtered.
(b) The Excipient Solution is added, with mixing, to a compounding vessel containing sialidase fusion protein (total protein comprising at least four subspecies of DAS181 as described in Section II.1 above) at 125 mg/ml initial concentration of total protein.
(c) Sterile filtered isopropanol is added to the compound vessel with mixing to form the Feedstock Solution. The final composition of the Feedstock Solution is as follows: 70 mg/ml sialidase fusion protein (total protein comprising at least four subspecies of DAS181 as described in Section II.1 above), 25% isopropanol, 4.99 mg/ml histidine, 6.80 mg/ml histidine-HCl, 10.50 mg/ml trehalose, 5.06 mg/ml magnesium sulfate, 0.21 mg calcium chloride, 0.05 mg/ml sodium acetate, 0.02 mg/ml acetic acid. The pH of the solution is 6.0. The time between initiating the addition of isopropanol and starting the lyophilization cycle is within 60 minutes.
(e) Stainless Steel trays that have undergone depyrogenation are each filled with 18 g of the Feedstock Solution, using a metering pump.
(f) The filled Stainless Steel trays are subjected to a Lyophilization Cycle as follows:
a. the trays are gasketed and placed in the lyophilizer shelves at 25° C. for 5 minutes;
b. the temperature of the shelves is lowered to −45° C. at a rate of −0.5° C./minute;
c. primary drying is accomplished by setting the condenser to less than −80° C., applying a vacuum of 125 mTorr and increasing the temperature to −0° C. at a ramp rate of 1° C./minute and then holding for 60 hrs;
d. the secondary drying is accomplished increasing the temperature to 30° C. at a rate of 1° C./minute and then holding for 6 hrs; and
e. the vacuum is released and the lyophilizer is backfilled with nitrogen to prevent oxidation of the microparticle formulation before transferring into bottles for bulk mixing and aliquoting the bulk powder for storage at ≤−15° C.

The microparticles of Formulation II can have one or more of: an MMAD of 6.5 microns (or 2-8 microns. 3-8 microns or 5-7), a GSD of 1.5-1.7 (or 1.3-1.9 or 1.4-1.8), a FPF (volume % below 5 microns) of 6.6% (less than 35%, 30%, 25%, 20%, 15%, 10% or 5%) and, a Tg of 38° C.

Thus, in some embodiments, the microparticles have a MMAD of 3-8 (5-7) microns (6.2-6.8 microns) with a GSD of 1.3-1-6 (1.4-1.6), a FPF of less than 9% (less than 8%, less than 7%, about 6-7%) and comprise (on a weight % basis) sialidase fusion protein (total protein comprising at least four subspecies of DAS181 as described in Section II. 1 above): 60-70% (62-68%, 64-66%, 65%); Histidine free base: 3-6% (4-5%); Histidine HCl: −5-9% (5-7%, 8-9%); Trehalose: 7-11% (8-10%, 8.5-9.5%); Magnesium sulfate: 4-8% (5-7%, 4-6%); and Water: 6-12% (8-12%, 9-11%). The microparticles can also include small amounts of sodium acetate (less than 1%, less than 0.5%, less than 0.1%, less than 0.05%, e.g., 0.03%); of calcium chloride (less than 1%, less than 0.5%, less than 0.4%, less than 0.3%, e.g., 0.1-0.3%) and small amounts of acetic acid (less than 1%, less than 0.5%, less than 0.1%, less than 0.05%, e.g., 0.01%). Small amounts of residual isopropanol can sometimes be present (less than 1%, less than 0.5%, less than 0.1%, less than 0.05%, less than 0.01%).

When the microparticles of Formulation II are anhydrous they can comprise (on a weight % basis) sialidase fusion protein (total protein comprising at least four subspecies of DAS181 as described in Section II. 1 above): 69-74% (70-73%, 71-72%, 72%); Histidine free base: 3-8% (4-7%, 4-6%); Histidine HCl: 4-9% (5-8%, 6-7%); Trehalose: 8-12% (9-11%, 8-10%); Magnesium sulfate: 4-8% (5-7%, 6-7%). The microparticles can also include small amounts of sodium acetate (less than 1%, less than 0.5%, less than 0.1%, less than 0.05%, e.g., 0.03%); small amounts of calcium chloride (less than 1%, less than 0.5%, less than 0.1%, less than 0.05%, e.g., 0.03%) and small amounts of acetic acid (less than 1%, less than 0.5%, less than 0.1%, less than 0.05%, e.g., 0.01%). Small amounts of residual isopropanol can sometimes be present (less than 1%, less than 0.5%, less than 0.1%, less than 0.05%, less than 0.01%).

### Microparticle Formulation I

In some embodiments, a sialidase fusion protein (total protein comprising at least four subspecies of DAS181 as described in Section II. 1 above) is provided in the following microparticle formulation (also referred herein as “Formulation I—not anhydrous”):
a) a sialidase fusion protein: 86.7% (w/w %) of total protein comprising at least four subspecies of DAS181 as described in Section II.1 above)
b) sodium sulfate: 2.5% (w/w %)
c) Water: 10.0% (w/w %, depending on humidity of storage conditions)

In some embodiments, the formulation can also include small amounts of sodium acetate (less than 1%, less than 0.5%, less than 0.1%, less than 0.05%, e.g., 0.03%); small amounts of calcium chloride (less than 1%, less than 0.5%, less than 0.1%, less than 0.05%, e.g., 0.03%) and small amounts of acetic acid (less than 1%, less than 0.5%, less than 0.1%, less than 0.05%, e.g., 0.01%). Small amounts of residual isopropanol can sometimes be present (less than 1%, less than 0.5%, less than 0.1%, less than 0.05%, less than 0.01%).

In some embodiments, there is provided a dry microparticle formulation (also referred herein as “Formulation I—anhydrous” comprising:
a) sialidase fusion protein: 96.3% (w/w %) total protein comprising at least four subspecies of DAS181 as described in Section II. 1 above
b) sodium sulfate 2.7% (w/w %)

The microparticles of Formulation I can also include small amounts of sodium acetate (less than 1%, less than 0.8%, less than 0.7%, less than 0.6%); small amounts of calcium chloride (less than 1%, less than 0.5%, less than 0.4%) and small amounts of acetic acid (less than 1%, less than 0.5%, less than 0.4%, less than 0.3%, e.g., 0.2%). Small amounts of residual isopropanol can sometimes be present (less than 1%, less than 0.5%, less than 0.1%, less than 0.05%, less than 0.01%).

The microparticles of Formulation I can have one or more of: an MMAD of 4-7 microns (or 2-8 microns. 3-8 microns or 5-7 microns), a GSD of 1.5-1.7 (or 1.3-1.9 or 1.4-1.8), a FPF (volume % below 5 microns) of 8% (less than 35%, 30%, 25%, 20%, 15%, 10% or 5%) and, a Tg of 38° C.

Thus, in some embodiments, the microparticles have a MMAD of 3-8 (5-7) microns (6.2-6.8 microns) with a GSD of 1.3-1-6 (1.4-1.6), a FPF of less than 9% (less than 8%, less than 7%, about 6-7%) and comprise (on a weight % basis) sialidase fusion protein (total protein comprising at least four subspecies of DAS181 as described in Section II.1 above):

The microparticle formulations (including, for example, Formulation I and Formulation II) obtained by the methods provided herein are of a relatively uniform size distribution, i.e., relatively monodisperse, with a geometric standard deviation (GSD) of between about 1.2 and 2.0, generally between about 1.2 and 1.5, 1.6, 1.7, or 1.8. The particles are also homogeneous, i.e., the formulation components are not segregated and are evenly distributed throughout the particles. Further, the fine particle fraction (FPF) containing microparticles that are smaller than 5 microns is less than 10%, generally less than about 8%, 7%, 6%, 5%, 4%, 3.5%, 3%, 2.5%, 2%, 1.5%, or 1%.

### III. Methods of Making and Assessing Compositions

In some aspects, the DAS181 compositions provided herein comprising at least four subspecies of DAS181 can be prepared according to standard methods of protein expression and purification. In some embodiments, the DAS181 compositions can be purified from cells comprising an expression vector encoding a sequence one or more of the DAS181 subspecies. In some embodiments, the DAS181 compositions can be purified from cells comprising an expression vector encoding the amino acid sequence of SEQ ID NO. 1. Overexpressing a protein in a cell (e.g., a bacterial cell) can be achieved using an expression vector. Expression vectors can be autonomous or integrative. A recombinant nucleic acid (e.g., one encoding the amino acid sequence of SEQ ID NO: 1, or an amino acid sequence having at least 90%, 95%, 96%, 97%, 98%, or 99% sequence identity to the amino acid sequence of SEQ ID NO: 1) can be introduced into the cell in the form of an expression vector such as a plasmid. The recombinant nucleic acid can be maintained extra chromosomally or it can be integrated into the chromosomal DNA. Expression vectors can contain selection marker genes encoding proteins required for cell viability under selected conditions (to permit detection and/or selection of those cells transformed with the desired nucleic acids. Expression vectors can also include an autonomous replication sequence (ARS).

Transformed cells (i.e., microbial cells) can be selected for by using appropriate techniques including, but not limited to, culturing auxotrophic cells after transformation in the absence of the biochemical product required, selection for and detection of a new phenotype, or culturing in the presence of an antibiotic which is toxic to the yeast in the absence of a resistance gene contained in the transformants. Transformants can also be selected and/or verified by integration of the expression cassette into the genome, which can be assessed by, e.g., Southern blot or PCR analysis. Prior to introducing the vectors into a target cell of interest, the vectors can be grown (e.g., amplified) in bacterial cells such as Escherichia coli (E. coli) or Pseudomonas fluorescens (P. fluorescens) as described above. Pseudomonas fluorescens expression systems have been described, for example, in U.S. Pat. Nos. 10,041,102 and 8,288,127, the contents of each of which are herein incorporated by reference in their entirety. The vector DNA can be isolated from bacterial cells by any of the methods known in the art which result in the purification of vector DNA from the bacterial milieu. The purified vector DNA can be extracted extensively with phenol, chloroform, and ether, to ensure that no microbial proteins are present in the plasmid DNA preparation, since these proteins can be toxic to mammalian cells.

Expression systems that can be used for small or large scale production of polypeptides include, without limitation, microorganisms such as bacteria (e.g., E. coli or P. fluorescens) transformed with recombinant bacteriophage DNA, plasmid DNA, or cosmid DNA expression vectors containing the nucleic acid molecules, and fungal (e.g., S. cerevisiae) transformed with recombinant fungal expression vectors containing the nucleic acid molecules.

In general, for in vivo production of a protein of interest by bacterial (e.g., E. coli or P. fluorescens) recombinant cells, the cells can be cultured in an aqueous nutrient medium comprising sources of assimilatable nitrogen and carbon, typically under submerged aerobic conditions (shaking culture, submerged culture, etc.). The aqueous medium can be maintained at a pH of 4.0-8.0 (e.g., 4.5, 5.0, 5.5, 6.0, or 7.5), using protein components in the medium, buffers incorporated into the medium or by external addition of acid or base as required. Suitable sources of carbon in the nutrient medium can include, for example, carbohydrates, lipids and organic acids such as glucose, sucrose, fructose, glycerol, starch, vegetable oils, petrochemical derived oils, succinate, formate, and the like. Suitable sources of nitrogen can include, for example, yeast extract, Corn Steep Liquor, meat extract, peptone, vegetable meals, distillers solubles, dried yeast, and the like as well as inorganic nitrogen sources such as ammonium sulphate, ammonium phosphate, nitrate salts, urea, amino acids, and the like.

Carbon and nitrogen sources, advantageously used in combination, need not be used in pure form because less pure materials, which contain traces of growth factors and considerable quantities of mineral nutrients, are also suitable for use. Desired mineral salts such as sodium or potassium phosphate, sodium or potassium chloride, magnesium salts, copper salts and the like can be added to the medium. An antifoam agent such as liquid paraffin or vegetable oils may be added in trace quantities as required but is not typically required.

Cultivation of recombinant cells (e.g., E. coli or P. fluorescens cells) expressing a protein of interest can be performed under conditions that promote optimal biomass and/or enzyme titer yields. Such conditions include, for example, batch, fed-batch or continuous culture. Further, changes to the parameters of the conditions can also promote optimal biomass and/or enzyme titer yields of the DAS181 protein. Such conditions include, for example, glycerol concentration in the culture media and high pO₂. For production of high amounts of biomass, submerged aerobic culture methods can be used, while smaller quantities can be cultured in shake flasks. For production in large tanks, a number of smaller inoculum tanks can be used to build the inoculum to a level high enough to minimize the lag time in the production vessel. The medium for production of the biocatalyst is generally sterilized (e.g., by autoclaving) prior to inoculation with the cells. Aeration and agitation of the culture can be achieved by mechanical means simultaneous addition of sterile air or by addition of air alone in a bubble reactor. A higher pO₂ (dissolved oxygen) can be used during cultivation in, for example, a bioreactor to promote optimal biomass. It can also be used to promote optimal active protein expression in the biomass culture. Implementation of such fermentation parameters, including a higher partial oxygen pressure and stepwise glycerol depletion, can result in an increased production of the protein in interest.

In some embodiments, provided herein is a method of releasing a composition of DAS181 subspecies, such as any one of the compositions described herein, for human medical use, comprising subjecting the composition to CEX-HPLC, and determining the relative amounts of the first, second, third, fourth, and optionally fifth subspecies separated by the CEX-HPLC. In some embodiments, the released composition for human medical use satisfies government regulatory standards, such as set by the U.S. FDA. In some embodiments, the CEX-HPLC comprises using non-porous 3 μm polystyrene divinylbenzene bead derivatized with carboxylic acid and elution by a gradient of up to 200 mM CaCl₂) in acetic buffer. In some embodiments, provided herein is a pharmaceutical composition released for human medical use by said method of release.

In some embodiments, provided herein is a method of assessing suitability of a composition of DAS181 for human medical use, comprising subjecting the composition to CEX-HPLC, and determining the relative amounts of the first, second, third, fourth, and optionally fifth subspecies separated by the CEX-HPLC. In some embodiments, the CEX-HPLC comprises using non-porous 3 μm polystyrene divinylbenzene bead derivatized with carboxylic acid and elution by a gradient of up to 200 mM CaCl₂) in acetic buffer. In some embodiments, provided herein is a pharmaceutical composition identified as suitable for human medical use by said method of assessing suitability. In some embodiments, the method comprises determining the weight percentage of the first, second, third, and fourth subspecies separated by the CEX-HPLC. In some embodiments, the method comprises identifying a composition of DAS181 as suitable for human medical use if the weight percentage of the first subspecies among the total proteins in the composition is at least 50%, the weight percentage of the second subspecies among the total proteins in the composition is less than or equal to 30% or about 30%, the weight percentage of the third subspecies among the total proteins in the composition is less than or equal to 15% or about 15%, and the weight percentage of the fourth subspecies among the total proteins in the composition is less than or equal to 4%.

In some embodiments, provided herein is a method of releasing a commercial batch of DAS181 for human medical use, comprising subjecting the composition to CEX-HPLC, and determining the relative amounts of the first, second, third, fourth, and optionally fifth subspecies separated by the CEX-HPLC. In some embodiments, the CEX-HPLC comprises using non-porous 3 μm polystyrene divinylbenzene bead derivatized with carboxylic acid and elution by a gradient of up to 200 mM CaCl₂ in acetic buffer. In some embodiments, provided herein is a commercial batch of DAS181 released by said method of release. In some embodiments, provided herein is a pharmaceutical composition released for human medical use by said method of release. In some embodiments, the method comprises releasing a commercial batch of DAS181 for human medical use if the weight percentage of the first subspecies among the total proteins in the composition is at least 50%, the weight percentage of the second subspecies among the total proteins in the composition is less than or equal to 30% or about 30%, the weight percentage of the third subspecies among the total proteins in the composition is less than or equal to 15% or about 15%, and the weight percentage of the fourth subspecies among the total proteins in the composition is less than or equal to 4%.

In some embodiments, a weight percentage of the first subspecies among the total proteins in the composition being at least 50%, a weight percentage of the second subspecies among the total proteins in the composition being about 5% to about 30%, a weight percentage of the third subspecies among the total proteins in the composition being about 1% to about 15%, and a weight percentage of the fourth subspecies among the total proteins in the composition being about 0.1% to about 15% is indicative of the suitability of the composition for human medical use.

In some embodiments, a weight percentage of the first subspecies among the total proteins in the composition being at least 50%, a weight percentage of the second subspecies among the total proteins in the composition being less than about 30%, a weight percentage of the third subspecies among the total proteins in the composition being less than about 15%, and a weight percentage of the fourth subspecies among the total proteins in the composition being less than about 15% is indicative of the suitability of the composition for human medical use.

In some embodiments, a weight percentage of the first subspecies among the total proteins in the composition being at least 50%, a weight percentage of the second subspecies among the total proteins in the composition being less than about 30%, a weight percentage of the third subspecies among the total proteins in the composition being less than about 15%, and a weight percentage of the fourth subspecies among the total proteins in the composition being less than about 10% is indicative of the suitability of the composition for human medical use.

In some embodiments, a weight percentage of the first subspecies among the total proteins in the composition being at least 50%, a weight percentage of the second subspecies among the total proteins in the composition being less than about 30%, a weight percentage of the third subspecies among the total proteins in the composition being less than about 15%, and a weight percentage of the fourth subspecies among the total proteins in the composition being less than about 5% is indicative of the suitability of the composition for human medical use.

In some embodiments, a weight percentage of the first subspecies among the total proteins in the composition being at least 50%, a weight percentage of the second subspecies among the total proteins in the composition being less than or equal to 30% or about 30%, a weight percentage of the third subspecies among the total proteins in the composition being less than or equal to 15% or about 15%, and a weight percentage of the fourth subspecies among the total proteins in the composition less than or equal to 4% is indicative of the suitability of the composition for human medical use.

In some embodiments, indicators that the composition is suitable for medical use further include a total protein concentration between 1.1 and 1.4 mg/g. In some embodiments, indicators that the composition is suitable for medical use further include a monomer peak area percentage out of total protein in the sample of at least 96% as identified by size exclusion chromatography and/or SDS-PAGE analysis. In some embodiments, indicators that the composition is suitable for human medical use include a multimer peak area percentage out of total protein in the sample of less than or equal to 4% as identified by size exclusion chromatography. In some embodiments, indicators that the composition is suitable for human medical use include that the sialidase activity of the total protein in the composition is 540-740 U/mg protein. One milliunit (mU) of sialidase activity is defined as the amount of enzyme that releases 1.0 nmole of 4-Mu from MuNaNa in 20 min at 25° C., pH 8.5 in 100 mmol glycine buffer containing 0.5% BSA. In some embodiments, indicators that the composition is suitable for human medical use include that a reconstituted liquid solution of the lyophilized composition is a clear, colorless solution with no visible residue or visible undissolved matter. In some embodiments, indicators that the composition is suitable for human medical use include that a reconstituted liquid solution of the lyophilized composition has an osmolality of 270-330 mOsm/kg. In some embodiments, indicators that the composition is suitable for human medical use include that a reconstituted liquid solution of the lyophilized composition has a pH of between 4.5 and 6.5. In some embodiments, indicators that the composition is suitable for human medical use include positive identification of a major band by SDS-PAGE that conforms to a reference standard, wherein the reference standard comprises a) a first subspecies comprising a first fusion protein comprising a sialidase domain fused via its C-terminus to a cationic domain, wherein the cationic domain comprises amino acids 395-415 of SEQ ID NO: 1; b) a second subspecies comprising a deaminated form of the first fusion protein, wherein the deaminated form of the first fusion protein comprises a deaminated N residue compared to the first fusion protein, and wherein the amino acid position is relative to SEQ ID NO: 1; c) a third subspecies comprising a second fusion protein comprising a sialidase domain fused via its C-terminus to a truncated cationic domain, wherein the truncated cationic domain comprises amino acids 395-397 of SEQ ID NO: 1; and d) a fourth subspecies comprising a dimerized form of the first fusion protein. In some embodiments, indicators that the composition is suitable for human medical use include that the relative Km and kcat to the reference standard is 70%-130%.

In some embodiments, determining that a composition is suitable for human medical use comprises determining whether a vial comprising a lyophilized cake formulation of the composition is suitable for human medical use. In some embodiments, indicators that the vial comprising the lyophilized cake formulation of the composition include that the DAS181 and DAS181 variant subspecies content per vial is 4.5 mg-5.5 mg as determined by high performance liquid chromatography. In some embodiments, indicators that the composition is suitable for human medical use include that the moisture content of the lyophilized cake is less than or equal to 4%. In some embodiments, indicators that the composition is suitable for human medical use include that the level of bacterial endotoxin is less than 4.0 EU/vial. In some embodiments, indicators that the composition is suitable for human medical use include that the number of particles greater than or equal to 25 μM in diameter is less than or equal to 600 per vial. In some embodiments, indicators that the composition is suitable for human medical use include that the number of particles greater than or equal to 10 μM in diameter is less than or equal to 6000 per vial.

In some aspects, provided herein is a method of making a pharmaceutical composition comprising a composition comprising DAS181 described herein, comprising: a) introducing a nucleic acid encoding a protein of SEQ ID NO: 1 into a bacterial host cell; b) expressing the protein encoded by the nucleic in the bacterial host cell; c) purifying the protein by chromatography to obtain a purified protein composition; and d) assessing suitability of the purified protein composition for human medical use, wherein the assessing comprises subjecting the purified protein composition to CEX-HPLC and determining the relative amounts of the first, second, third, fourth, and optionally fifth subspecies separated by the CEX-HPLC. In some embodiments, the chromatography comprises cation-exchange chromatography (e.g., CEX-HPLC). In some embodiments, the chromatography comprises hydrophobic interaction chromatography (HIC). In some embodiments, the method further comprises formulating the purified protein composition to obtain a pharmaceutical composition. In some embodiments, the step of formulating the purified protein composition is carried out after the step of assessing suitability of the purified protein composition. In other embodiments, the step of formulating the purified protein composition is carried out before the step of assessing suitability of the purified protein composition. In some aspects, provided herein is a pharmaceutical composition made according to said method of making a pharmaceutical composition.

Methods of expressing sialidase fusion proteins in bacteria and purifying said sialidase fusion proteins have been described, for example, in U.S. Pat. No. 10,351,828, the content of which is herein incorporated by reference in its entirety.

### IV. Pharmaceutical Compositions, Kits and Articles of Manufacture

Further provided by the present application are pharmaceutical compositions, kits, and articles of manufacture comprising the compositions described herein.

In some aspects, provided herein is a pharmaceutical composition comprising at least four subspecies of DAS181 that can be separated by CEX-HPLC is provided, wherein the composition comprises: a) a first subspecies comprising a first fusion protein comprising a sialidase domain fused via its C-terminus to a cationic domain, wherein the cationic domain comprises amino acids 395-415 of SEQ ID NO: 1; b) a second subspecies comprising a deaminated form of the first fusion protein, wherein the deaminated form of the first fusion protein comprises a deaminated N residue compared to the first fusion protein, and wherein the amino acid position is relative to SEQ ID NO: 1; c) a third subspecies comprising a second fusion protein comprising a sialidase domain fused via its C-terminus to a truncated cationic domain, wherein the truncated cationic domain comprises amino acids 395-406 of SEQ ID NO: 1; and d) a fourth subspecies comprising a dimerized form of the first fusion protein, wherein the pharmaceutical composition is formulated according to any of the formulations described in Section II.2 above. In some embodiments, the formulation is a dry powder microparticle formulation. In some embodiments, the formulation is a lyophilized cake formulation. In some embodiments, the formulation is reconstituted in aqueous medium.

In some embodiments, the pharmaceutical composition comprises at least about 90% Trehalose by dry weight. In some embodiments, the pharmaceutical composition comprises at least about 0.2% MgSO₄ by dry weight. In some embodiments, the pharmaceutical composition comprises a) about 95-98% trehalose; b) about 0.2-0.4 MgSO₄; c) about 0.4-0.6% sodium acetate; and d) about 0.1-0.3% acetic acid.

In some embodiments, provided herein is a pharmaceutical composition comprising: a) a fusion protein comprising a sialidase domain fused at its C-terminus to a cationic domain; b) about 95-98% trehalose; c) about 0.2-0.4 MgSO₄; d) about 0.4-0.6% sodium acetate; and d) about 0.1-0.3% acetic acid.

In some embodiments, the composition does not comprise histidine or CaCl₂). In some embodiments, the pharmaceutical composition comprises about 97.5% Trehalose, about 0.3% MgSO₄, about 0.5% sodium acetate, and about 0.2% acetic acid.

In some embodiments, the composition does not comprise histidine or CaCl₂).

In some embodiments, the potency of the sialidase in the pharmaceutical composition is about 540-740 U/mg protein. In some embodiments, the pharmaceutical composition upon reconstitution into a liquid formulation has an osmolality of about 270-330 mOsm/kg. In some embodiments, the pharmaceutical composition upon reconstitution into a liquid formulation has a viscosity of about 1.27-1.39 cps. In some embodiments, the pharmaceutical composition upon reconstitution into a liquid formulation has a pH of about 4.5 to about 6.5. In some embodiments, at least about 95% of the proteins in the pharmaceutical composition are monomers.

Pharmaceutical compositions can be prepared by mixing the DAS181 compositions described herein having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers (e.g. sodium chloride), stabilizers, metal complexes (e.g. Zn-protein complexes); chelating agents such as EDTA and/or non-ionic surfactants.

The formulation can include a carrier. The carrier is a macromolecule which is soluble in the circulatory system and which is physiologically acceptable where physiological acceptance means that those of skill in the art would accept injection of said carrier into a patient as part of a therapeutic regime. The carrier preferably is relatively stable in the circulatory system with an acceptable plasma half-life for clearance. Such macromolecules include but are not limited to soy lecithin, oleic acid and sorbitan trioleate.

The formulations can also include other agents useful for pH maintenance, solution stabilization, or for the regulation of osmotic pressure. Examples of the agents include but are not limited to salts, such as sodium chloride, or potassium chloride, and carbohydrates, such as glucose, galactose or mannose, and the like.

In some embodiments, the pharmaceutical composition is contained in a single-use vial, such as a single-use sealed vial. In some embodiments, the pharmaceutical composition is contained in a multi-use vial. In some embodiments, the pharmaceutical composition is contained in bulk in a container. In some embodiments, the pharmaceutical composition is cryopreserved.

In some embodiments, the systems provided herein can be stably stored under cryopreservation conditions, such as, for example, at −80° C., and can be thawed as needed or desired prior to administration. For example, the systems provided herein can be stored at a preserving temperature, such as −20° C. or −80° C., for at least or between about a few hours, 1, 2, 3, 4, or 5 hours, or days, including at least or between about a few years, such as, but not limited to, 1, 2, 3, or more years, for example for at least or about 1, 2, 3, 4, or 5 hours to at least or about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 hours or 4, 5, 6, 7, 8, 9, 10, 15, 20, 25, or 30 days or 1.5, 2, 2.5, 3, 3.5, 4, 4.5, 5, 5.5, 6, 6.5, 7, 7.5, 8, 8.5, 9, 9.5, 10, 10.5, 11, 11.5, or 12 months or 1, 2, 3, 4, or 5 or more years prior to thawing for administration. The systems provided herein also stably can be stored under refrigeration conditions such as, at 4° C. and/or transported on ice to the site of administration for treatment. For example, the systems provided herein can be stored at 4° C. or on ice for at least or between about a few hours, such as, but not limited to, 1, 2, 3, 4, or 5 hours, to at least or about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 or more hours prior to administration for treatment.

The present application further provides kits and articles of manufacture for use in any embodiment of the treatment methods described herein. The kits and articles of manufacture may comprise any one of the formulations and pharmaceutical compositions described herein.

In some embodiments, there is provided a kit comprising an immunoconjugate described herein and instructions for administering the immunoconjugate to an individual. In some embodiments, the kit further comprises instructions for treating a cancer.

In some embodiments, there is provided a kit comprising any one of the immunoconjugates described herein, and instructions for treating a cancer. In some embodiments, the kit further comprises an immunotherapeutic agent (e.g., a cell therapy or any one of the immunotherapies described herein). In some embodiments, the kit further comprises one or more additional therapeutic agents for treating the cancer. In some embodiments, the immunoconjugate and/or the one or more immunotherapeutic agents are in a single composition (e.g., a composition comprising an immune checkpoint inhibitor and an immunoconjugate). In some embodiments, the immunoconjugate and optionally the one or more additional immunotherapeutic agents and/or additional therapeutic agents for treating the cancer are in separate compositions.

The kits of the invention are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

In some aspects, provided herein is a vial comprising any one of the pharmaceutical compositions described herein. In some embodiments, the vial is for single use.

In some aspects, provided herein is a nebulizer comprising a liquid formulation, wherein the liquid formulation is reconstituted from any of the lyophilized cake pharmaceutical compositions described herein. Dosage forms or administration by nebulizers generally contain large amounts of water in addition to the active ingredient. Minor amounts of other ingredients such as pH adjusters, emulsifiers or dispersing agents, preservatives, surfactants, or buffering and other stabilizing and solubilizing agents can also be present.

Nasal formulations can be administered as drops, sprays, aerosols or by any other intranasal dosage form. Optionally, the delivery system can be a unit dose delivery system. The volume of solution or suspension delivered per dose can be anywhere from about 5 to about 2000 microliters, from about 10 to about 1000 microliters, or from about 50 to about 500 microliters. Delivery systems for these various dosage forms can be dropper bottles, plastic squeeze units, atomizers, nebulizers or pharmaceutical aerosols in either unit dose or multiple dose packages.

The liquid formulations disclosed herein can be varied to include; (1) other acids and bases to adjust the pH; (2) other tonicity imparting agents such as sorbitol, glycerin and dextrose; (3) other antimicrobial preservatives such as other parahydroxy benzoic acid esters, sorbate, benzoate, propionate, chlorobutanol, phenylethyl alcohol, benzalkonium chloride, and mercurials; (4) other viscosity imparting agents such as sodium carboxymethylcellulose, microcrystalline cellulose, polyvinylpyrrolidone, polyvinyl alcohol and other gums; (5) suitable absorption enhancers; (6) stabilizing agents such as antioxidants, like bisulfite and ascorbate, metal chelating agents such as sodium edetate and drug solubility enhancers such as polyethylene glycols; and (7) other agents such as amino acids.

In some aspects, provided herein are includes liquid pharmaceutical compositions at various dosage levels, such as dosage levels of DAS181 active agent (or another polypeptide having sialidase activity) between about 0.01 mg and about 100 mg. Examples of such dosage levels include doses of about 0.05 mg, 0.06 mg, 0.1 mg, 0.5 mg, 1 mg, 5 mg, 10 mg, 20 mg, 50 mg, or 100 mg/day. The foregoing doses can be administered one or more times per day, for one day, two days, three days, four days, five days, six days, seven days, eight days, nine days, ten days, eleven days, twelve days, thirteen days, or fourteen or more days. Higher doses or lower doses can also be administered. Typically, dosages can be between about 1 ng/kg and about 10 mg/kg, between about 10 ng/kg and about 1 mg/kg, and between about 100 ng/kg and about 100 micrograms/kg. In various examples described herein, mice were treated with various dosages of the compositions described herein, including dosages of 0.0008 mg/kg, 0.004 mg/kg, 0.02 mg/kg, 0.06 mg/kg, 0.1 mg/kg, 0.3 mg/kg, 0.6 mg/kg, 1.0 gm/kg, 2.0 mg/kg, 3.0 mg/kg, 4.0 mg/kg, and 5.0 mg/kg.

Also provided herein are articles of manufacture that contain a microparticle formulation as described in Section II.2.A that includes a sialidase or a sialidase fusion protein in an amount of about 60% to about 75% w/w, about 95-98% w/w trehalose, about 0.2-0.4% w/w MgSO₄, about 0.4-0.6% w/w sodium acetate, about 0.1-0.3% w/w acetic acid, a packaging material for the formulation, and a label that indicates that the composition is for a therapeutic indication. In one embodiment, the therapeutic indication is influenza. In other embodiments, the therapeutic indication is asthma or COPD. In yet other embodiments, the therapeutic indication is selected from among parainfluenza, RSV, sinusitis, otitis, laryngitis, bronchitis, pneumonia, bronchiectasis, vasculitis, mucous plugging, Wegener's granulomatosis, and cystic fibrosis (CF).

In some embodiments, the scavenging agent is histidine; in other embodiments, the scavenging agent is a combination of histidine and trehalose. In yet other embodiments, the counterion is selected from among citric acid/citrate, magnesium sulfate, potassium sulfate or calcium sulfate, phosphate, pivalate, rubidium, bromine, perchlorate, itaconate, and any salt, acid, or base form thereof.

The packaging material can be a HPMC capsule; in further embodiments, the HPMC capsule is clear. In other embodiments, the packaging material can be a gel capsule, or a pullulan polysaccharide capsule. In yet other embodiments, the article of manufacture further contains a secondary packaging material; in some embodiments, the secondary packaging material is a foil laminate; in particular embodiments, the foil laminate is a cold form foil aluminum laminate blister pack. In some embodiments, the scavenging agent is an amine; in further embodiments, the amine is selected from among lysine, histidine, glycine, arginine, glutamine, glutamic acid, cysteine, alanine, tyrosine, tryptophan, aminoguanidine, cysteamine, serine, carnosine, hydralazine, and poly(l-lysine).

In some embodiments, the scavenging agent is histidine or a combination of histidine and trehalose. In some embodiments, the article of manufacture comprises a package insert comprising instructions for reconstituting a lyophilized cake formulation described herein into a liquid formulation for administration via a nebulizer (e.g., a vibrating mesh nebulizer). In some embodiments, the liquid formulation liquid formulation comprises 1.3 mg/mL DAS181 and DAS181 variant subspecies, 94 mg/mL trehalose, 0.3 mg/mL MgSO₄, 0.5 mg/mL sodium acetate, and 0.2 mg/mL acetic acid.

The articles of manufacture provided herein also can contain an inhaler for pulmonary administration of the composition. In certain embodiments, the inhaler is a dry powder inhaler, a metered dose inhaler or an electrostatic delivery device.

Also provided herein are articles of manufacture that contain a lyophilized cake formulation as described in Section II.2.A that includes a sialidase or a sialidase fusion protein in an amount of about 1% w/w to 2% w/w, a counterion and a scavenging agent that is a primary amine in the amount of about 8% w/w to about 11% w/w, a packaging material for the formulation, and a label that indicates that the composition is for a therapeutic indication. In some embodiments, the packaging material is a sealed glass vial. In some embodiments, the article of manufacture includes a label providing instructions for reconstitution of the lyophilized cake formulation in aqueous medium. In some embodiments, the target inhalation volume is 3.5 mL, and the reconstitution volume for the single glass vial is 3.8 mL. In one embodiment, the therapeutic indication is influenza. In other embodiments, the therapeutic indication is asthma or COPD. In yet other embodiments, the therapeutic indication is selected from among parainfluenza, RSV, sinusitis, otitis, laryngitis, bronchitis, pneumonia, bronchiectasis, vasculitis, mucous plugging, Wegener's granulomatosis, and cystic fibrosis (CF). In some embodiments, the article of manufacture further contains a secondary packaging material. In some embodiments, the secondary packaging material is a container comprising multiple sealed glass vials.

The articles of manufacture provided herein also can contain a nebulizer for pulmonary administration of the composition.

### V. Methods of Treatment

In some aspects, provided herein is a method of treating a disease or condition in an individual comprising administering to the individual an effective amount of any of the pharmaceutical compositions described herein.

In some embodiments, provided herein is a method of treating a disease or condition in an individual comprising administering to the individual an effective amount of a pharmaceutical composition comprising at least four subspecies of DAS181 that can be separated by CEX-HPLC is provided, wherein the composition comprises: a) a first subspecies comprising a first fusion protein comprising a sialidase domain fused via its C-terminus to a cationic domain, wherein the cationic domain comprises amino acids 395-415 of SEQ ID NO: 1; b) a second subspecies comprising a deaminated form of the first fusion protein, wherein the deaminated form of the first fusion protein comprises a deaminated N residue compared to the first fusion protein, and wherein the amino acid position is relative to SEQ ID NO: 1; c) a third subspecies comprising a second fusion protein comprising a sialidase domain fused via its C-terminus to a truncated cationic domain, wherein the truncated cationic domain comprises amino acids 395-406 of SEQ ID NO: 1; and d) a fourth subspecies comprising a dimerized form of the first fusion protein. In some embodiments, the pharmaceutical composition is formulated according to any of the formulations described in Section II.2 above. In some embodiments, the formulation is a dry powder microparticle formulation. In some embodiments, the formulation is a lyophilized cake formulation. In some embodiments, the formulation is reconstituted in aqueous medium.

In some embodiments, provided herein is a method of treating a disease or condition in an individual comprising administering to the individual an effective amount of a pharmaceutical composition comprising: a) a fusion protein comprising a sialidase domain fused at its C-terminus to a cationic domain; b) about 95-98% w/w trehalose; c) about 0.2-0.4% w/w MgSO₄; d) about 0.4-0.6% w/w sodium acetate; and e) about 0.1-0.3% w/w acetic acid by dry weight. In some embodiments, the pharmaceutical composition comprises about 97.5% w/w Trehalose, about 0.3% w/w MgSO₄, about 0.5% w/w sodium acetate, and about 0.2% w/w acetic acid by dry weight. In some embodiments, the pharmaceutical composition is a liquid composition reconstituted from a lyophilized composition. In some embodiments, the liquid formulation comprises 1-2 mg/mL DAS181 and DAS181 variant subspecies, 10-100 mg/mL trehalose, 0.1-0.5 mg/mL MgSO₄, 0.2-0.7 mg/mL sodium acetate, and 0.1-0.3 mg/mL acetic acid. In some embodiments the liquid formulation comprises about 1.3 mg/mL DAS181 and DAS181 variant subspecies, about 94 mg/mL trehalose, about 0.3 mg/mL MgSO₄, about 0.5 mg/mL sodium acetate, and about 0.2 mg/mL acetic acid. In some embodiments the liquid formulation comprises 1.3 mg/mL DAS181 and DAS181 variant subspecies, 94 mg/mL trehalose, 0.3 mg/mL MgSO₄, 0.5 mg/mL sodium acetate, and 0.2 mg/mL acetic acid. In some embodiments, the fusion protein is DAS181, whose sequence is set forth in SEQ ID NO: 1 (amino terminal methionine present) and SEQ ID NO: 2 (no amino terminal methionine). In some embodiments, the fusion protein is A) a polypeptide comprising (or consisting of or consisting essentially of) the amino acid sequence of SEQ ID NO: 1; B) a polypeptide comprising (or consisting of or consisting essentially of) the amino acid sequence of SEQ ID NO: 2; or C) a mixture of a polypeptides comprising (or consisting of or consisting essentially of) SEQ ID NO: 1 and polypeptides comprising (or consisting of or consisting essentially of) SEQ ID NO: 2.

In some embodiments, provided herein is a method of treating a disease or condition in an individual comprising administering to the individual an effective amount of a pharmaceutical composition comprising: i) a mixture of DAS181 subspecies that can be separated by CEX-HPLC, comprising a) a first subspecies comprising a first fusion protein comprising a sialidase domain fused via its C-terminus to a cationic domain, wherein the cationic domain comprises amino acids 395-415 of SEQ ID NO: 1; b) a second subspecies comprising a deaminated form of the first fusion protein, wherein the deaminated form of the first fusion protein comprises a deaminated N residue compared to the first fusion protein, and wherein the amino acid position is relative to SEQ ID NO: 1; c) a third subspecies comprising a second fusion protein comprising a sialidase domain fused via its C-terminus to a truncated cationic domain, wherein the truncated cationic domain comprises amino acids 395-406 of SEQ ID NO: 1; and d) a fourth subspecies comprising a dimerized form of the first fusion protein fusion protein comprising a sialidase domain fused at its C-terminus to a cationic domain; ii) about 95-98% w/w trehalose; iii) about 0.2-0.4% w/w MgSO₄; iv) about 0.4-0.6% w/w sodium acetate; and v) about 0.1-0.3% w/w acetic acid by dry weight. In some embodiments, the pharmaceutical composition comprises about 97.5% w/w Trehalose, about 0.3% w/w MgSO₄, about 0.5% w/w sodium acetate, and about 0.2% w/w acetic acid by dry weight. In some embodiments, the pharmaceutical composition is a liquid composition reconstituted from a lyophilized composition. In some embodiments, the liquid formulation comprises 1-2 mg/mL DAS181 and DAS181 variant subspecies, 10-100 mg/mL trehalose, 0.1-0.5 mg/mL MgSO₄, 0.2-0.7 mg/mL sodium acetate, and 0.1-0.3 mg/mL acetic acid. In some embodiments the liquid formulation comprises about 1.3 mg/mL DAS181 and DAS181 variant subspecies, about 94 mg/mL trehalose, about 0.3 mg/mL MgSO₄, about 0.5 mg/mL sodium acetate, and about 0.2 mg/mL acetic acid. In some embodiments the liquid formulation comprises 1.3 mg/mL DAS181 and DAS181 variant subspecies, 94 mg/mL trehalose, 0.3 mg/mL MgSO₄, 0.5 mg/mL sodium acetate, and 0.2 mg/mL acetic acid.

In some embodiments, the disease or condition is a viral infection or is associated with a viral infection. In some embodiments, the viral infection is a coronavirus infection (e.g., SARS-CoV, MERS-COV, or SARS-COV-2), influenza infection, lower Tract parainfluenza infection, BK polyomavirus, Merkel Cell Polyomavirus (MCPyV), or Human enterovirus D68 (EV-D68) infection. DAS181 has been shown to have potent antiviral activity against all forms of influenza, including pandemic strains such as H1N1 and avian flu. DAS181 is also active against drug-resistant IFV, including strains resistant to Tamiflu (oseltamivir) and Relenza (zanamivir). Asian lineage avian influenza A (H7N9) virus and two additional respiratory viruses that cause serious illness and depend on SA to infect cells, metapneumovirus and enterovirus 68, are also inhibited by DAS181.

In some embodiments, provided herein is a method for prevention, prophylaxis or treatment of diseases of the respiratory tract including influenza, parainfluenza, RSV, sinusitis, otitis, laryngitis, bronchitis, pneumonia, allergic and non-allergic asthma, COPD, bronchiectasis, vasculitis, mucous plugging, Wegener's granulomatosis, and cystic fibrosis (CF), said method comprising administering any of the pharmaceutical compositions described herein to a patient in need thereof.

Therapeutic and diagnostic applications of the pharmaceutical compositions disclosed herein can include drug delivery, vaccination, gene therapy, and in vivo tissue or tumor imaging. Routes of administration can include oral or parenteral administration; mucosal administration; ophthalmic administration; intravenous, subcutaneous, intra-articular, or intramuscular injection; inhalation administration; and topical administration.

In some embodiments, provided herein is a method for treating parainfluenza virus (PIV) or influenza virus (IFV) infection in a patient, the method comprising: administering to the respiratory tract of the patient a therapeutically effective amount of any of the pharmaceutical compositions disclosed herein. Also described herein is a method for treating a subject at risk for PIV or IFV infection, the method comprising: administering to the respiratory tract of the subject any of the pharmaceutical compositions described herein. Methods of treating or preventing PIV or IFV infections comprising administering a protein having sialidase activity are described in US20150132274, the content of which is herein incorporated by reference in its entirety. In various cases: the patient is an immunocompromised patient; the patient is suffering from a primary immunodeficiency; the immunocompromised patient is suffering from a secondary immunodeficiency; the immunocompromised patient is being or has been treated with an immunosuppressive therapy; the immunocompromised patient is being or has been treated with a chemotherapeutic agent; the immunocompromised patient is a transplant patient; the composition further comprises one or more additional compounds; the administration is by use of a dry powder inhaler; the administration is by use of a nasal spray; the administration is by use of a nebulizer; the administration is by use of an endotracheal tube (ET tube), and a dry powder inhaler. In some cases the patient has insufficient pulmonary function to make effective use of dry powder inhaler or unable to use dry powder inhaler at all, e.g. patients on mechanical ventilator. In some cases the patient is an immunocompromised patient infected with PIV and is treated with a liquid formulation (e.g., using a nebulizer) or is treated with a dry formulation (e.g., using a dry powder inhaler). In some embodiments, the pharmaceutical composition comprises i) a mixture of DAS181 subspecies that can be separated by CEX-HPLC, comprising a) a first subspecies comprising a first fusion protein comprising a sialidase domain fused via its C-terminus to a cationic domain, wherein the cationic domain comprises amino acids 395-415 of SEQ ID NO: 1; b) a second subspecies comprising a deaminated form of the first fusion protein, wherein the deaminated form of the first fusion protein comprises a deaminated N residue compared to the first fusion protein, and wherein the amino acid position is relative to SEQ ID NO: 1; c) a third subspecies comprising a second fusion protein comprising a sialidase domain fused via its C-terminus to a truncated cationic domain, wherein the truncated cationic domain comprises amino acids 395-406 of SEQ ID NO: 1; and d) a fourth subspecies comprising a dimerized form of the first fusion protein fusion protein comprising a sialidase domain fused at its C-terminus to a cationic domain.

In some aspects, provided herein are methods of treating an infection by BK polyomavirus or a BK polyomavirus associated disorder in a patient, the method comprising administering to the patient a therapeutically effective amount of an agent having sialidase activity in any of the pharmaceutical compositions disclosed herein. Methods for treating an infection by BK polyomavirus or a BK polyomavirus associated disorder comprising administering a protein having sialidase activity are described in U.S. Pat. No. 10,300,116, the content of which is herein incorporated by reference in its entirety. In various embodiments: the patient is immunocompromised; the patient has undergone haematopoietic stem cell transplant or is being prepared for haematopoietic stem cell transplant; the disorder is BKV nephropathy; the disorder is nephritis; disorder is hemorrhagic cystitis; the disorder is ureteral stenosis; the patient has undergone solid organ transplant or is being treated in preparation for solid organ transplant; the disorder is lupus. In some embodiments, the pharmaceutical composition comprises i) a mixture of DAS181 subspecies that can be separated by CEX-HPLC, comprising a) a first subspecies comprising a first fusion protein comprising a sialidase domain fused via its C-terminus to a cationic domain, wherein the cationic domain comprises amino acids 395-415 of SEQ ID NO: 1; b) a second subspecies comprising a deaminated form of the first fusion protein, wherein the deaminated form of the first fusion protein comprises a deaminated N residue compared to the first fusion protein, and wherein the amino acid position is relative to SEQ ID NO: 1; c) a third subspecies comprising a second fusion protein comprising a sialidase domain fused via its C-terminus to a truncated cationic domain, wherein the truncated cationic domain comprises amino acids 395-406 of SEQ ID NO: 1; and d) a fourth subspecies comprising a dimerized form of the first fusion protein fusion protein comprising a sialidase domain fused at its C-terminus to a cationic domain. In some embodiments, the pharmaceutical composition comprises about 97.5% w/w Trehalose, about 0.3% w/w MgSO₄, about 0.5% w/w sodium acetate, and about 0.2% w/w acetic acid by dry weight. In some embodiments, the pharmaceutical composition is a liquid composition reconstituted from a lyophilized composition. In some embodiments, the liquid formulation comprises 1-2 mg/mL DAS181 and DAS181 variant subspecies, 10-100 mg/mL trehalose, 0.1-0.5 mg/mL MgSO₄, 0.2-0.7 mg/mL sodium acetate, and 0.1-0.3 mg/mL acetic acid. In some embodiments the liquid formulation comprises about 1.3 mg/mL DAS181 and DAS181 variant subspecies), about 94 mg/mL trehalose, about 0.3 mg/mL MgSO₄, about 0.5 mg/mL sodium acetate, and about 0.2 mg/mL acetic acid. In some embodiments the liquid formulation comprises 1.3 mg/mL DAS181 and DAS181 variant subspecies, 94 mg/mL trehalose, 0.3 mg/mL MgSO₄, 0.5 mg/mL sodium acetate, and 0.2 mg/mL acetic acid.

In some embodiments, provided herein is a method of treating an infection by a Merkel Cell Polyomavirus (MCPyV) or MCPyV related disorder, the method comprising administering to the skin of the patient a therapeutically effective amount of an agent having sialidase activity in any of the pharmaceutical compositions disclosed herein. Methods for treating MCPyV infection or MCPyV related disorders comprising administering a protein having sialidase activity are described in US20200222511, the content of which is herein incorporated by reference in its entirety. In various embodiments: the patient is immunocompromised; the patient is infected with HIV; the patient is suffering from chronic lymphocytic leukemia; the patient has undergone organ transplant or is being treated in preparation for organ transplant; the patient has undergone liver, heart, bone marrow or kidney transplant or is being treated in preparation for liver, heart, bone marrow or kidney transplant. In some embodiments, the pharmaceutical composition comprises i) a mixture of DAS181 subspecies that can be separated by CEX-HPLC, comprising a) a first subspecies comprising a first fusion protein comprising a sialidase domain fused via its C-terminus to a cationic domain, wherein the cationic domain comprises amino acids 395-415 of SEQ ID NO: 1; b) a second subspecies comprising a deaminated form of the first fusion protein, wherein the deaminated form of the first fusion protein comprises a deaminated N residue compared to the first fusion protein, and wherein the amino acid position is relative to SEQ ID NO: 1; c) a third subspecies comprising a second fusion protein comprising a sialidase domain fused via its C-terminus to a truncated cationic domain, wherein the truncated cationic domain comprises amino acids 395-406 of SEQ ID NO: 1; and d) a fourth subspecies comprising a dimerized form of the first fusion protein fusion protein comprising a sialidase domain fused at its C-terminus to a cationic domain. In some embodiments, the pharmaceutical composition comprises about 97.5% w/w Trehalose, about 0.3% w/w MgSO₄, about 0.5% w/w sodium acetate, and about 0.2% w/w acetic acid by dry weight. In some embodiments, the pharmaceutical composition is a liquid composition reconstituted from a lyophilized composition. In some embodiments, the liquid formulation comprises 1-2 mg/mL DAS181 and DAS181 variant subspecies, 10-100 mg/mL trehalose, 0.1-0.5 mg/mL MgSO₄, 0.2-0.7 mg/mL sodium acetate, and 0.1-0.3 mg/mL acetic acid. In some embodiments the liquid formulation comprises about 1.3 mg/mL DAS181 and DAS181 variant subspecies, about 94 mg/mL trehalose, about 0.3 mg/mL MgSO₄, about 0.5 mg/mL sodium acetate, and about 0.2 mg/mL acetic acid. In some embodiments the liquid formulation comprises 1.3 mg/mL DAS181 and DAS181 variant subspecies, 94 mg/mL trehalose, 0.3 mg/mL MgSO₄, 0.5 mg/mL sodium acetate, and 0.2 mg/mL acetic acid.

In some embodiments, provided herein are methods of treating an infection by MERS-COV or a MERS-COV associated disorder in a patient, the method comprising administering to the patient a therapeutically effective amount of an agent having sialidase activity in any of the pharmaceutical compositions disclosed herein. Methods for treating an infection caused by MERS-COV or a MERS-COV associated disorder in a patient comprising administering a protein having sialidase activity are described in US20200397871, the content of which is herein incorporated by reference in its entirety. In various embodiments: the patient is immunocompromised; the patient is undergoing immunosuppressive therapy; and/or the patient is over age 70. In some embodiments, the pharmaceutical composition comprises i) a mixture of DAS181 subspecies that can be separated by CEX-HPLC, comprising a) a first subspecies comprising a first fusion protein comprising a sialidase domain fused via its C-terminus to a cationic domain, wherein the cationic domain comprises amino acids 395-415 of SEQ ID NO: 1; b) a second subspecies comprising a deaminated form of the first fusion protein, wherein the deaminated form of the first fusion protein comprises a deaminated N residue compared to the first fusion protein, and wherein the amino acid position is relative to SEQ ID NO: 1; c) a third subspecies comprising a second fusion protein comprising a sialidase domain fused via its C-terminus to a truncated cationic domain, wherein the truncated cationic domain comprises amino acids 395-406 of SEQ ID NO: 1; and d) a fourth subspecies comprising a dimerized form of the first fusion protein fusion protein comprising a sialidase domain fused at its C-terminus to a cationic domain. In some embodiments, the pharmaceutical composition comprises about 97.5% w/w Trehalose, about 0.3% w/w MgSO₄, about 0.5% w/w sodium acetate, and about 0.2% w/w acetic acid by dry weight. In some embodiments, the pharmaceutical composition is a liquid composition reconstituted from a lyophilized composition. In some embodiments, the liquid formulation comprises 1-2 mg/mL DAS181 and DAS181 variant subspecies, 10-100 mg/mL trehalose, 0.1-0.5 mg/mL MgSO₄, 0.2-0.7 mg/mL sodium acetate, and 0.1-0.3 mg/mL acetic acid. In some embodiments the liquid formulation comprises about 1.3 mg/mL DAS181 and DAS181 variant subspecies, about 94 mg/mL trehalose, about 0.3 mg/mL MgSO₄, about 0.5 mg/mL sodium acetate, and about 0.2 mg/ml acetic acid. In some embodiments the liquid formulation comprises 1.3 mg/mL DAS181 and DAS181 variant subspecies, 94 mg/mL trehalose, 0.3 mg/mL MgSO₄, 0.5 mg/mL sodium acetate, and 0.2 mg/mL acetic acid.

In some embodiments, provided herein is a method of treating an infection by a Human enterovirus D68 (EV-D68) (species, Human enterovirus D; genus, Enterovirus; family, Picornaviridae) or an EV-D68 associated disorder in a patient, the method comprising administering to the patient a therapeutically effective amount of an agent having sialidase activity in any of the pharmaceutical compositions disclosed herein. Methods for treating an EV-D68 infection or an EV-D68 associated disorder in a patient comprising administering a protein having sialidase activity are described in U.S. Pat. No. 10,328,128, the content of which is herein incorporated by reference in its entirety. In various embodiments: the patient is immunocompromised; the patient is undergoing immunosuppressive therapy; the patient is over age 70; the patient is under age 18; or the patient is under age 10. In some embodiments, the pharmaceutical composition comprises i) a mixture of DAS181 subspecies that can be separated by CEX-HPLC, comprising a) a first subspecies comprising a first fusion protein comprising a sialidase domain fused via its C-terminus to a cationic domain, wherein the cationic domain comprises amino acids 395-415 of SEQ ID NO: 1; b) a second subspecies comprising a deaminated form of the first fusion protein, wherein the deaminated form of the first fusion protein comprises a deaminated N residue compared to the first fusion protein, and wherein the amino acid position is relative to SEQ ID NO: 1; c) a third subspecies comprising a second fusion protein comprising a sialidase domain fused via its C-terminus to a truncated cationic domain, wherein the truncated cationic domain comprises amino acids 395-406 of SEQ ID NO: 1; and d) a fourth subspecies comprising a dimerized form of the first fusion protein fusion protein comprising a sialidase domain fused at its C-terminus to a cationic domain. In some embodiments, the pharmaceutical composition comprises about 97.5% w/w Trehalose, about 0.3% w/w MgSO₄, about 0.5% w/w sodium acetate, and about 0.2% w/w acetic acid by dry weight. In some embodiments, the pharmaceutical composition is a liquid composition reconstituted from a lyophilized composition. In some embodiments, the liquid formulation comprises 1-2 mg/mL DAS181 and DAS181 variant subspecies, 10-100 mg/mL trehalose, 0.1-0.5 mg/mL MgSO₄, 0.2-0.7 mg/mL sodium acetate, and 0.1-0.3 mg/mL acetic acid. In some embodiments the liquid formulation comprises about 1.3 mg/mL DAS181 and DAS181 variant subspecies, about 94 mg/mL trehalose, about 0.3 mg/mL MgSO₄, about 0.5 mg/mL sodium acetate, and about 0.2 mg/mL acetic acid. In some embodiments the liquid formulation comprises 1.3 mg/mL DAS181 and DAS181 variant subspecies, 94 mg/mL trehalose, 0.3 mg/mL MgSO₄, 0.5 mg/mL sodium acetate, and 0.2 mg/mL acetic acid.

In some embodiments, the method comprises administering the pharmaceutical composition by inhalation. For administration by inhalation, the pharmaceutical composition can be delivered in the form of an aerosol spray from a pressured container or dispenser that contains a suitable propellant, e.g., a gas such as carbon dioxide, or a nebulizer. Such methods include those described in U.S. Pat. No. 6,468,798. In some embodiments, the agent(s) are administered to the subject by using a nebulizer, a vaporizer, a nasal sprayer, a pressurized metered dose inhaler, or a breath activated pressurized metered dose inhaler.

In some embodiments, the device is a small, hard bottle to which a metered dose sprayer is attached. The metered dose can be delivered by drawing the composition into a chamber of defined volume, which chamber has an aperture dimensioned to aerosolize and aerosol formulation by forming a spray when a liquid in the chamber is compressed. The chamber is compressed to administer the composition. In certain devices, the chamber is a piston arrangement. Such devices are commercially available.

Alternatively, a squeeze bottle with an aperture or opening dimensioned to aerosolize an aerosol formulation by forming a spray when squeezed can be used. The opening is usually found in the top of the bottle, and the top is generally tapered to partially fit in the nasal passages for efficient administration of the aerosol formulation. Preferably, the nasal inhaler can provide a metered amount of the aerosol formulation, for administration of a measured dose of the therapeutic agent.

In some embodiments, the method comprises administering the pharmaceutical composition using a nebulizer, such as a vibrating mesh nebulizer. In some embodiments, the total protein (DAS181, or DAS181 and DAS181 variant subspecies) added to the nebulizer is between 0.65 and 4.5 mg/day. In some embodiments, the total protein (DAS181, or DAS181 and DAS181 variant subspecies) dose added to the nebulizer is 4.5 mg/day for an adult patient (e.g., a patient age 18 or above). In some embodiments, the total protein (DAS181, or DAS181 and DAS181 variant subspecies) dose added to the nebulizer is 2.5 mg/day for a pediatric patient. In some embodiments, a pediatric patient is a patient having weight <40 kg.

While certain embodiments have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### EXAMPLES

### Example 1. CEX-HPLC Characterization of Composition Comprising DAS181 Subspecies

This example describes characterization of a composition comprising DAS181 subspecies by CEX-HPLC.

A standard lyophilization manufacturing process was developed to generate a sterile lyophilized cake comprising DAS181 subspecies in a glass vial configuration. Lyophilization parameters including freezing and heating ramp, primary and secondary drying temperature and time, vacuum setting are evaluated and selected based on the finished product cake appearance.

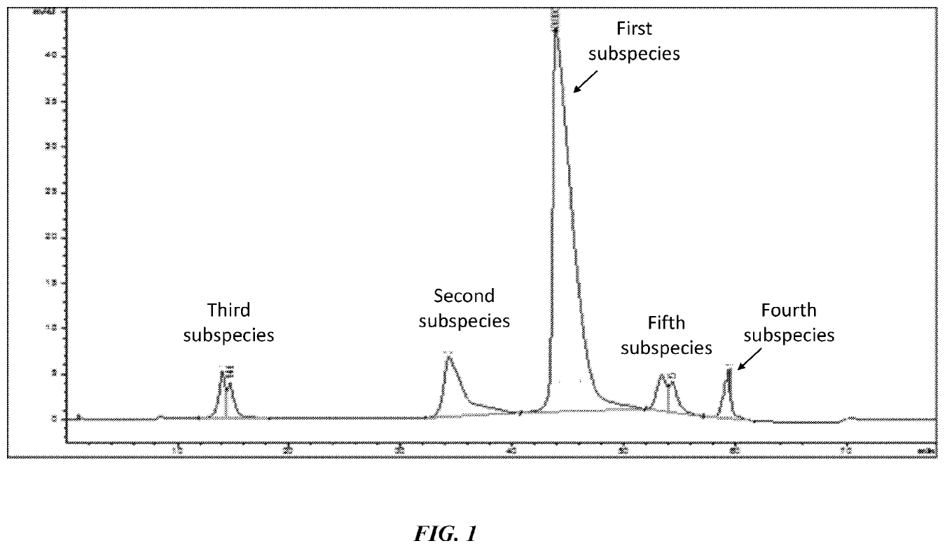

The composition was subjected to cation-exchange chromatography (CEX-HPLC) using non-porous 3 μm polystyene divinylbenzene bead derivatized with carboxylic acid and elution by a gradient of up to 200 mM CaCl₂ in acetic buffer at a flow rate of 1 mL/min. The results of the CEX-HPLC are shown in FIG. 1.

As shown in FIG. 1, the composition comprised at least four subspecies, which include DAS181 subspecies that can be separated by CEX-HPLC. The first subspecies eluted at about 84 mM CaCl₂). The second subspecies eluted at about 72 mM CaCl₂). The third subspecies eluted at about 57.5 mM CaCl₂). The fourth subspecies eluted at about 200 mM CaCl₂. The composition further comprised a fifth subspecies, which was not isolated for further analysis. The fifth subspecies eluted at about 100 mM CaCl₂.

### Example 2. Characterization of DAS181 Subspecies

This example describes intact mass analysis of the first, second, third, and fourth DAS181 subspecies identified by CEX-HPLC.

About 5 μg of sample was loaded onto the SEC-MS column for analysis. An Agilent 6550 iFunnel QTOF was used for MS data collection. Agilent BioConfirm Software was used for MS analysis.

The SEC-MS method was performed using the following parameters: Isocratic; Mobile Phase: 30% w/v Acetonitrile, 0.1% w/v formic Acid in LC/MS Water; Column: Waters BEH SEC Column, 200 A, 1.7 μm, 4.6×300 mm.

The deconvolution parameters for the intact mass analysis were as follows: Deconvolution algorithm: Maximum Entropy; Mass Range: 30 kDa-150 kDa; Mass Step: 0.5 Da; M/Z Range: 650-4000 Da; Baseline Factor: 7.0; Adduct: Proton; Isotope Width: automatic.

### First Subspecies

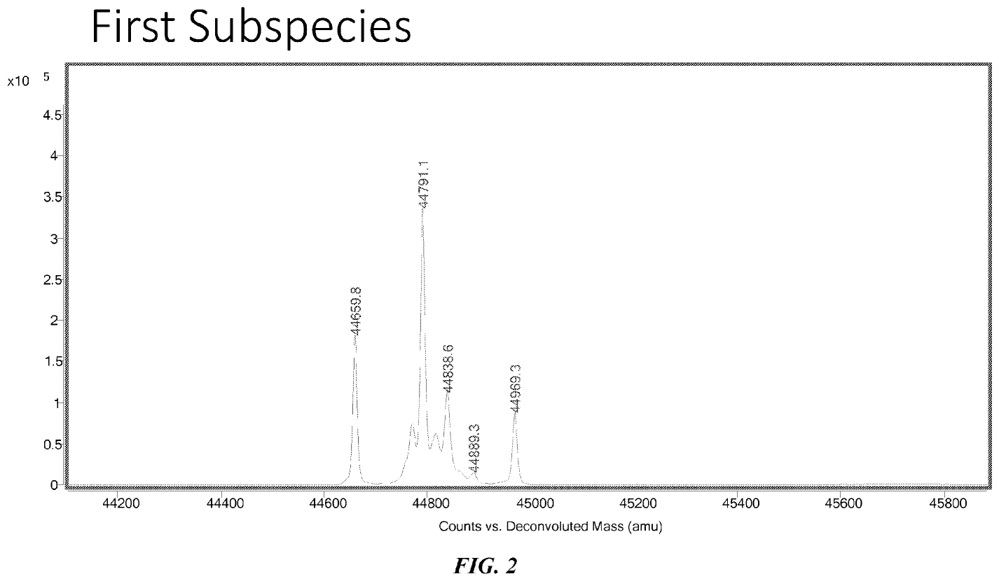

The results of the intact mass analysis for the first species are shown in FIG. 2 and Table E1 below.

TABLE E1
First Subspecies peaks identified by intact mass analysis.

| Species (amino acid | | Experimental | Mass |
|---|---|---|---|
| numbering based | Theoretical | Mass | Difference |
| on SEQ ID NO: 1) | Mass (Da) | (Da) | (Da) |
| DAS181 (G2-P415) | 44657.42 | 44659.80 | 2.38 |
| DAS181 (M1-P415) | 44788.61 | 44791.10 | 2.49 |
| DAS 181 (G2-P415) + | 44835.42 | 44838.60 | 3.18 |
| 178 Da* | | | |
| DAS181 (M1-P415) + | 44966.61 | 44969.30 | 3.67 |
| 178 Da | | | |

### Second Subspecies

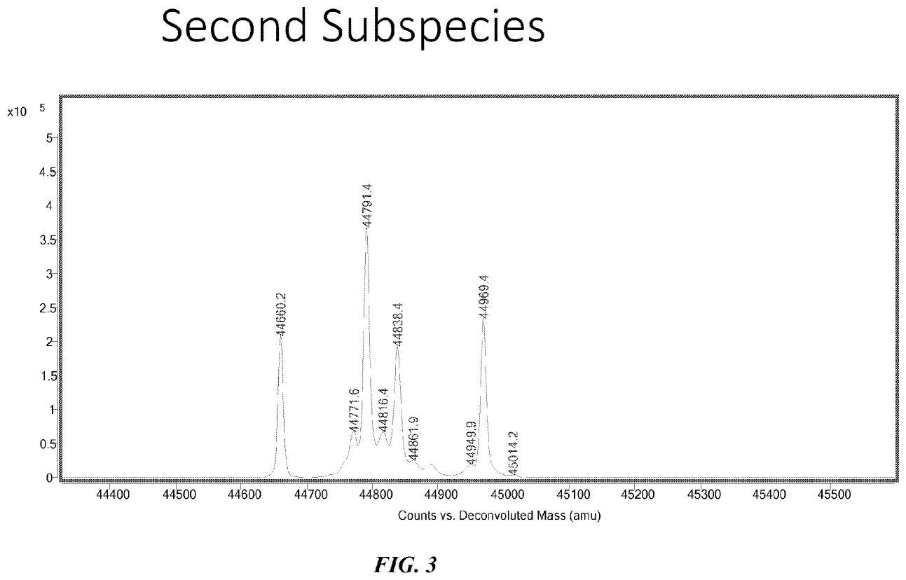

The results of the intact mass analysis for the first species are shown in FIG. 3 and Table E2 below.

TABLE E2
Second Subspecies peaks identified by intact mass analysis.

| Species (amino acid | | Experimental | Mass |
|---|---|---|---|
| numbering based | Theoretical | Mass | Difference |
| on SEQ ID NO: 1) | Mass (Da) | (Da) | (Da) |
| DAS181 (G2-P415) | 44657.42 | 44660.20 | 2.78 |
| DAS181 (M1-P415) | 44788.61 | 44791.40 | 2.79 |
| DAS 181 (G2-P415) + 178 Da* | 44835.42 | 44838.40 | 2.98 |
| DAS181 (M1-P415) + 178 Da | 44966.61 | 44969.40 | 2.79 |

### Third Subspecies

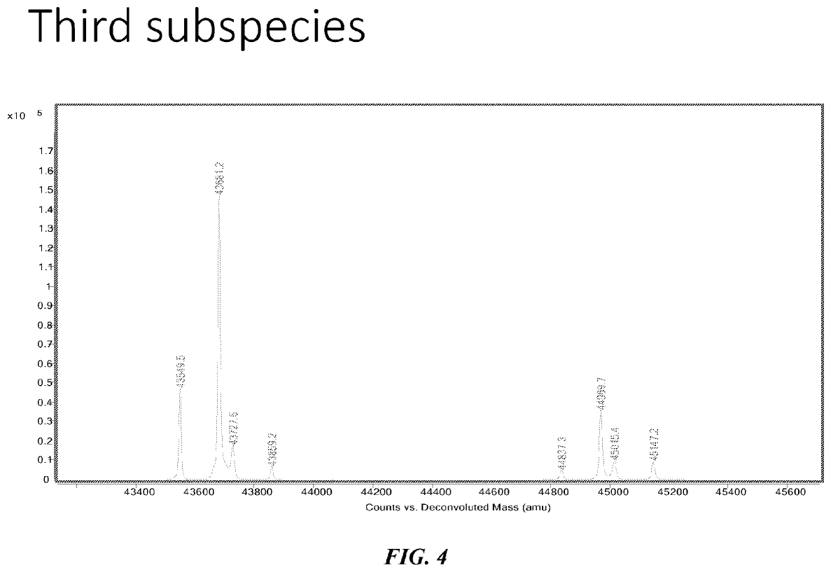

The results of the intact mass analysis for the first species are shown in FIG. 4 and Table E3 below.

TABLE E3
Third subspecies peaks identified by intact mass analysis.

| Peak | Experimental Mass (Da) |
|---|---|
| 1 | 43549.50 |
| 2 | 43681.20 |
| 3 | 43727.50 |
| 4 | 43859.20 |
| 5 | 44837.30 |
| 6 | 44969.70 |
| 7 | 45015.40 |
| 8 | 45147.20 |

### Fourth Subspecies

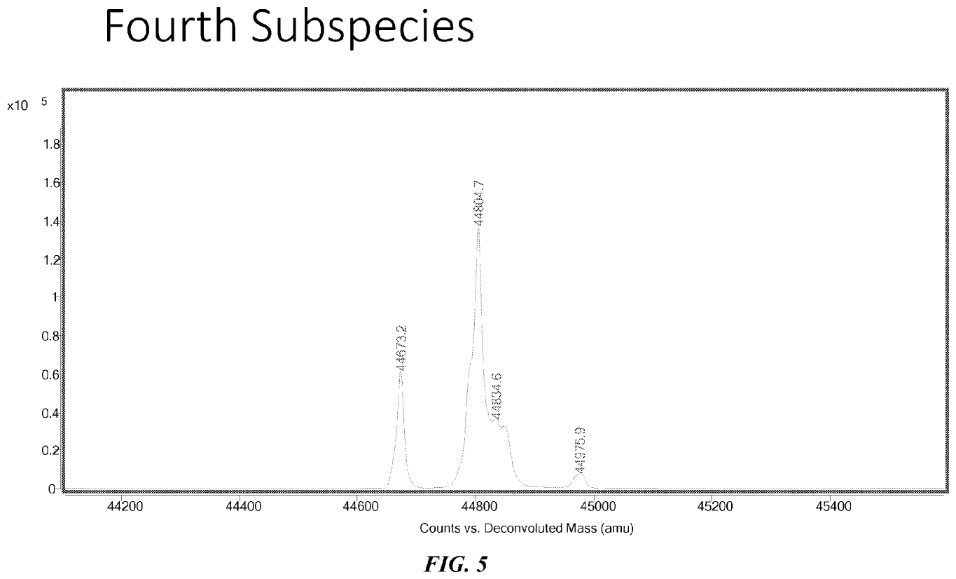

The results of the intact mass analysis for the first species are shown in FIG. 5 and Table E4 below.

TABLE E5
Fourth subspecies peaks identified by intact mass analysis.

| | | | Mass |
|---|---|---|---|
| Species (amino acid numbering | Theoretical | Experimental | Difference |
| based on SEQ ID NO: 1) | Mass (Da) | Mass (Da) | (Da) |
| DAS181 (G2-P415) + | 44673.42 | 44673.20 | 0.22 |
| Methionine Oxidation | | | |
| DAS181 (M1-P415) + | 44804.61 | 44804.70 | 0.09 |
| Methionine Oxidation | | | |
| Unknown (peak 3) | — | 44975.90 | — |

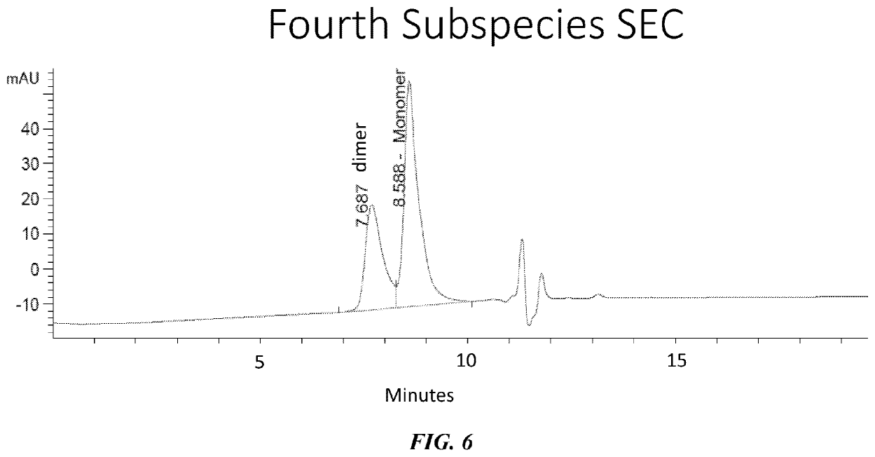

The fourth subspecies was analyzed by size exclusion chromatography high performance liquid chromatography (SEC-HPLC) to identify the oligomeric state of the proteins in the fourth subspecies. The results of the SEC-HPLC experiment are provided in FIG. 6, and quantification of the peaks is provided in Table E6 below. Based on SEC, the fourth subspecies comprised about 35% dimer and about 64% monomer by weight.

TABLE E6
Fourth subspecies SEC-HPLC analysis.

| | Retention | Width | | | Oligomeric |
|---|---|---|---|---|---|
| Peak # | Time [min] | [min] | Area | Area % | state |
| 1 | 7.687 | 44673.20 | 878.558 | 35.180 | Dimer |
| 2 | 8.588 | 44804.70 | 1618.799 | 64.820 | Monomer |

### Size Variant Distribution by SDS-PAGE

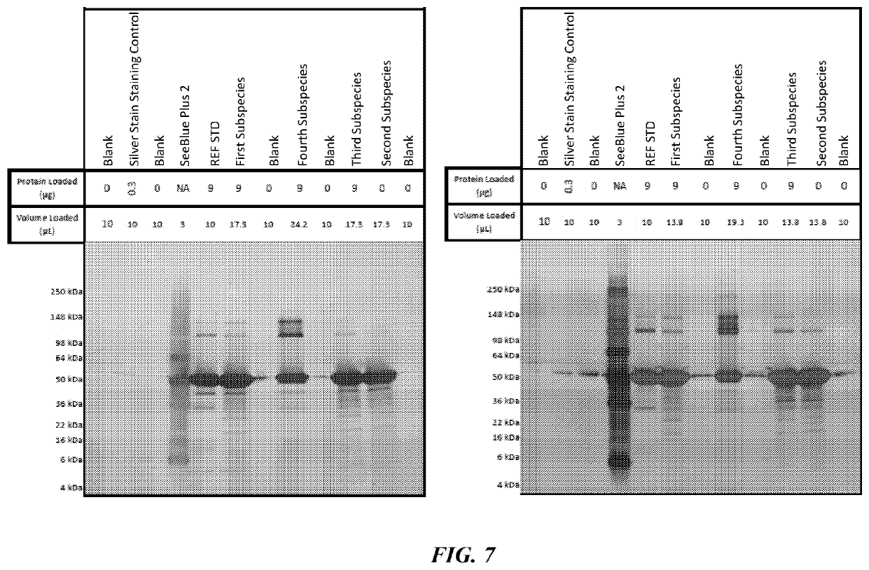

As shown in FIG. 7, the identity of DAS181 subspecies was confirmed by SDS-PAGE with Coomassie blue stain detection by comparison to the Reference Standard. The main band positions of all the drug product lots detected by Coomassie stain were consistent with the main band of DAS181 reference standard. The minor bands observed on the reduced silver stained SDS-PAGE are all DAS181 related, and their differences were due to the drug substance lot-to-lot variability. The minor differences observed on the silver stained SDS-PAGE gel are not likely to have any impact to product safety and efficacy.

### Example 3. Activity of DAS181 Subspecies

This example demonstrates the sialidase activity of the isolated first, second, third, and fourth DAS181 subspecies. Sialidase activity was determined using 2′-(4-methylumbelliferyl)-α-D-N-acetylneuraminic acid (MuNaNa), VWR (Biosynth) Cat #101369-938 (M-5507), a fluorogenic substrate. The sialidase (DAS181 subspecies) cleaves the sialic acid from the substrate, and the assay detects free sialic acid. As shown in Table E7 below, all DAS181 subspecies retained sialidase activity. Results are shown for two different purification lots measured in two independent experiments. The control for sialidase activity measurements was the first fusion protein.

TABLE E6
Sialidase activity of DAS181 subspecies

| Purification | | Sialidase Activity |
|---|---|---|
| Lot | Subspecies | relative to Control (%) |
| 1 | First subspecies | 144% |
| | Second subspecies | 93% |
| | Third subspecies | 100% |
| | Fourth subspecies | 100% |
| 2 | First subspecies | 94% |
| | Second subspecies | 86% |
| | Third subspecies | 88% |
| | Fourth subspecies | 47% |

### Example 4. Lyophilized Cake Formulation of DAS181

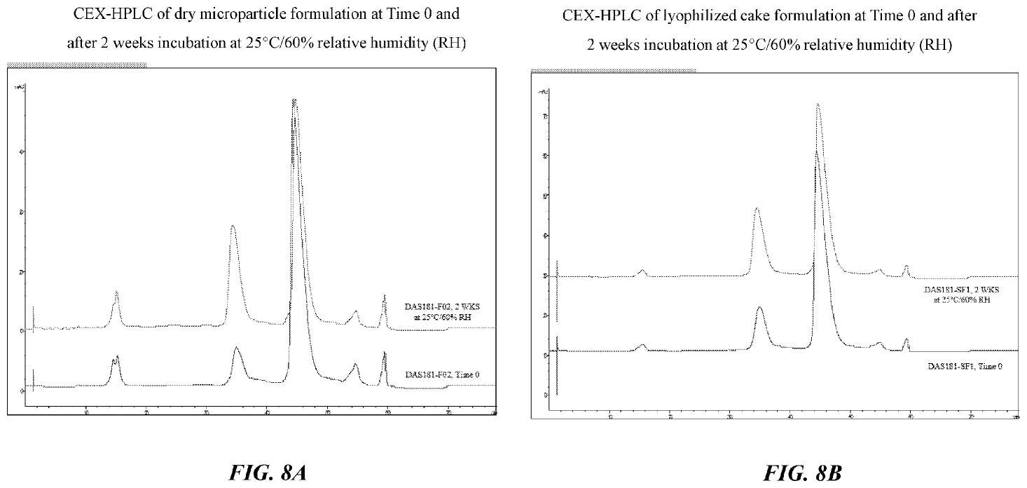

In this comparability study, reconstituted dry lyophilized microparticle formulations (based on previously described formulations in U.S. Pat. No. 9,700,602) and the new lyophilized cake formulation disclosed herein were tested after storage at 25° C./60% RH for up to 2-weeks. The Initial (Time 0), 24-hour, and 1-week time point samples were pulled and stored at −80° C. until the 2-week time point samples were pulled. All samples were tested side-by-side. The most sensitive stability-indicating methods for DAS181 were used for this comparability assessment, which include purity by CEX-HPLC and % Monomer by SEC-HPLC. Deamidation is the major degradation pathway of DAS181 in solution. The second subspecies is primarily a deamidated product of Peak A and retains full potency. Percent Second Subspecies in the lyophilized cake formulation is 16.8% at Time 0 and 28.0% at 2-weeks. Percent Second Subspecies in the dry microparticle formulation is 10.8% at Time 0 and 28.8% at 2-weeks. The increase of percent Peak C in the reconstituted DAS181-SF1 lot was 11.2% in 2-weeks, whereas the increase of percent Second Subspecies in reconstituted dry microparticle formulation was 18.0% in 2-weeks. Therefore, the reconstituted lyophilized cake formulation is more stable than the reconstituted dry microparticle formulation when stored at 25° C./60% RH. Representative CEX-HPLC chromatograms for the reconstituted dry microparticle formulation and reconstituted lyophilized cake formulation are shown in FIG. 8A and FIG. 8B, respectively. Table E8 below provides quantification of the peak areas at different timepoints. Table E9 provides SEC-HPLC and activity measurements for the lyophilized cake formulation after storage at room temperature for 6-8 hours, 24 hours (plus or minus 1 hour), 48 hours (plus or minus 1 hour), 3 days, or 7 days.

TABLE E7
CEX-HPLC Results of Reconstituted dry microparticle formulation
and lyophilized cake formulation lots at 25° C./60%
RH (% shown for subspecies out of total protein by weight)

| | | % Peak Area | | | |
|---|---|---|---|---|---|
| Product Lot | Peak | Time 0 | 24 Hours | 1 Week | 2 Weeks |
| Dry | First Subspecies | 78.0% | 77.1% | 72.6% | 67.1% |
| microparticle | Second Subspecies | 16.8% | 17.7% | 22.3% | 28.0% |
| formulation | Third Subspecies | 1.7% | 1.7% | 1.7% | 1.7% |
| | Fourth Subspecies | 1.6% | 1.6% | 1.6% | 1.5% |
| | Fifth Subspecies | 2.0% | 2.0% | 1.9% | 1.7% |
| Lyophilized | First Subspecies | 76.7% | 75.0% | 67.2% | 59.8% |
| cake | Second Subspecies | 10.8% | 12.8% | 20.9% | 28.8% |
| formulation | Third Subspecies | 5.8% | 5.7% | 5.8% | 5.7% |
| | Fourth Subspecies | 3.0% | 3.0% | 2.9% | 2.9% |
| | Fifth Subspecies | 3.7% | 3.6% | 3.1% | 2.8% |

TABLE E8
SEC-HPLC and activity measurements for lyophilized cake formulation lots at 25° C./60% RH

| | | | Time Points | | | | | |
|---|---|---|---|---|---|---|---|---|
| | | | Time 0 | 6-8 | 24 ± 1 | 48 ± 1 | | |
| Test | Target | | (Initial) | Hours | Hours | Hours | 3 Days | 7 Days |
| Protein | Report Results | 1.1-1.4 mg/g | 1.19 | 1.19 | 1.18 | 1.17 | 1.17 | 1.17 |
| Concentration | | | | | | | | |
| by UV | | | | | | | | |
| Sialidase | Report Results | 540-740 U/mg | 670 | 680 | 689 | 689 | 696 | 695 |
| Activity | | protein | | | | | | |
| % Monomer by | Report Results | | | | | | | |
| Size Exclusion | Monomer Peak | ≥96.0% | 99.9% | 99.9% | 99.9% | 99.9% | 99.9% | 99.9% |
| HPLC | Area % | | | | | | | |
| | Multimer Peak | ≤4.0% | 0.1% | 0.1% | 0.1% | 0.1% | 0.1% | 0.1% |
| | Area % | | | | | | | |
| | Single Unknown | ≤0.5% | Not | Not | Not | Not | Not | Not |
| | Peak Area % | | Detected | Detected | Detected | Detected | Detected | Detected |
| | Total Unknown | ≤2.0% | Not | Not | Not | Not | Not | Not |
| | Peak Area % | | Detected | Detected | Detected | Detected | Detected | Detected |
| Purity by | Report Results | | | | | | | |
| CEX-HPLC | First Subspecies | ≥50% | 82.39% | 82.20% | 82.00% | 81.49% | 81.23% | 79.62% |
| | Second Subspecies | Report | 10.01% | 10.19% | 10.42% | 10.89% | 11.12% | 12.82% |
| | Third Subspecies | Results | 4.18% | 4.17% | 4.16% | 4.15% | 4.20% | 4.13% |
| | Fifth Subspecies | | 2.02% | 2.04% | 2.04% | 2.02% | 2.05% | 2.01% |
| | Fourth Subspecies | ≤4.0% | 1.40% | 1.39% | 1.39% | 1.44% | 1.40% | 1.42% |
| | Other Peaks | Report | Not | Not | Not | Not | Not | Not |
| | | Results | Detected | Detected | Detected | Detected | Detected | Detected |

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

### Example 5. Updated Lyophilization Cycle for DAS181 Lyophilized Cake Formulation

This example describes an updated lyophilization cycle for DAS181 formulation into using an increased primary drying temperature and a reduced overall lyophilization time from 103 hours to 63 hours (e.g., see Table E9) compared to previously described lyophilization cycles (e.g., see U.S. Pat. No. 9,700,602, the content of which is herein incorporated by reference in its entirety). The resulting product was a white, lyophilized cake that formed a clear, colorless solution with no visible residue or undissolved matter upon reconstitution.

TABLE E9
Updated lyophilization cycle for DAS181 formulation
Updated Cycle Parameter

| Step | | Temp./ | | | |
|---|---|---|---|---|---|
| # | Operation | Vacuum | | Time | |
| 1 | Load set point | 20° | C. | Maintain | |
| | | | | shelf temp | |
| | | | | before | |
| | | | | loading | |
| 2 | Ramp shelf to | −45° | C. | 65 | minutes |
| 3 | Hold shelf at | −45° | C. | 180 | minutes |
| 4 | Set condenser | −65° | C. | N/A | |
| | Set Point | | | | |
| 5 | Set vacuum | 100 | mTorr | N/A | |
| | control | | | | |
| 6 | Ramp shelf to | −10° | C. | 30 | minutes |
| 7 | Hold shelf at | −10° | C. | 5 | minutes |
| 8 | Ramp shelf to | −25° | C. | 360 | minutes |
| 9 | Hold shelf at | −25° | C. | 1500 | minutes |
| 10 | Ramp shelf to | −10° | C. | 150 | minutes |
| 11 | Hold shelf at | −10° | C. | 510 | minutes |
| 12 | Ramp shelf to | 20° | C. | 300 | minutes |
| 13 | Hold shelf at | 20° | C. | 510 | minutes |

EXEMPLARY SEQUENCES

SEQ ID NO: 1 DAS181
MGDHPQATPAPAPDASTELPASMSQAQHLAANTATDNYRIPAITTAPNGD
LLISYDERPKDNGNGGSDAPNPNHIVQRRSTDGGKTWSAPTYIHQGTETG
KKVGYSDPSYVVDHQTGTIFNFHVKSYDQGWGGSRGGTDPENRGIIQAEV
STSTDNGWTWTHRTITADITKDKPWTARFAASGQGIQIQHGPHAGRLVQQ
YTIRTAGGAVQAVSVYSDDHGKTWQAGTPIGTGMDENKVVELSDGSLMLN
SRASDGSGFRKVAHSTDGGQTWSEPVSDKNLPDSVDNAQIIRAFPNAAPD
DPRAKVLLLSHSPNPRPWSRDRGTISMSCDDGASWTTSKVFHEPFVGYTT
IAVQSDGSIGLLSEDAHNGADYGGIWYRNFTMNWLGEQCGQKPAKRKKKG
GKNGKNRRNRKKKNP
SEQ ID NO: 2 DAS181 without N-terminal Met
GDHPQATPAPAPDASTELPASMSQAQHLAANTATDNYRIPAITTAPNGDL
LISYDERPKDNGNGGSDAPNPNHIVQRRSTDGGKTWSAPTYIHQGTETGK
KVGYSDPSYVVDHQTGTIFNFHVKSYDQGWGGSRGGTDPENRGIIQAEVS
TSTDNGWTWTHRTITADITKDKPWTARFAASGQGIQIQHGPHAGRLVQQY
TIRTAGGAVQAVSVYSDDHGKTWQAGTPIGTGMDENKVVELSDGSLMLNS
RASDGSGFRKVAHSTDGGQTWSEPVSDKNLPDSVDNAQIIRAFPNAAPDD
PRAKVLLLSHSPNPRPWSRDRGTISMSCDDGASWTTSKVFHEPFVGYTTI
AVQSDGSIGLLSEDAHNGADYGGIWYRNFTMNWLGEQCGQKPAKRKKKGG
KNGKNRRNRKKKNP

What is claimed is:

1. A composition comprising at least four subspecies of DAS181 that can be separated by cation exchange high performance liquid chromatography (CEX-HPLC), wherein the composition comprises:
a) a first subspecies comprising a first fusion protein comprising a sialidase domain fused via its C-terminus to a cationic domain, wherein the cationic domain comprises amino acids 395-415 of SEQ ID NO: 1;
b) a second subspecies comprising a deaminated form of the first fusion protein, wherein the deaminated form of the first fusion protein comprises a deaminated N residue compared to the first fusion protein, and wherein the amino acid position is relative to SEQ ID NO: 1;
c) a third subspecies comprising a second fusion protein comprising a sialidase domain fused via its C-terminus to a truncated cationic domain, wherein the truncated cationic domain comprises amino acids 395-397 of SEQ ID NO: 1; and
d) a fourth subspecies comprising a dimerized form of the first fusion protein.

2. The composition of claim 1, wherein the deaminated N residue in the second subspecies is N403.

3. The composition of claim 1 or 2, wherein the fourth subspecies comprises a methionine oxidized form of the first fusion protein.

4. The composition of any one of claims 1-3, further comprising a fifth subspecies comprising a dehydrated form of the first fusion protein.

5. The composition of any of claims 1-4, wherein the dimerized form of the first fusion protein has at least about 40% sialidase activity compared to that of the first fusion protein.

6. The composition of any one of claims 1-5, wherein the proteins in the third subspecies have at least about 80% sialidase activity comparing to that of the first fusion protein.

7. The composition of any one of claims 1-6, wherein the third subspecies further comprises oxidized or deaminated forms of the second fusion protein.

8. The composition of any one of claims 1-7, wherein the proteins in the first subspecies have at least about 94% sialidase activity comparing to that of the first fusion protein.

9. The composition of any one of claims 1-8, wherein the first subspecies further comprises a third fusion protein that lacks the N-terminal M residue comparing to the first fusion protein.

10. The composition of claim 9, wherein the ratio of the first fusion protein and the third fusion protein in the first subspecies is about 2:1.

11. The composition of any one of claims 1-10, wherein the proteins in the second subspecies have at least about 80% sialidase activity comparing to that of the first fusion protein.

12. The composition of any one of claims 1-11, wherein the sialidase domain comprises amino acids 1-394 of SEQ ID NO: 1.

13. The composition of any one of claims 1-12, wherein the first fusion protein comprises amino acids 1-415 of SEQ ID NO: 1.

14. The composition of any one of claims 1-13, wherein the second fusion protein comprises amino acids 1-406 of SEQ ID NO: 1.

15. The composition of any one of claims 1-14, wherein the weight percentage of the first subspecies among the total proteins in the composition is about 50% to about 90%.

16. The composition of any one of claims 1-15, wherein the weight percentage of the second subspecies among the total proteins in the composition is about 10% to about 30%.

17. The composition of any one of claims 1-16, wherein the weight percentage of the third subspecies among the total proteins in the composition is about 1% to about 15%.

18. The composition of any one of claims 1-17, wherein the weight percentage of the fourth subspecies among the total proteins in the composition is about 0.1% to about 4%.

19. The composition of any one of claims 1-18, wherein the weight percentage of the fifth subspecies among the total proteins in the composition is about 1% to about 10%.

20. The composition of any one of claims 1-19, wherein when the composition is subject to cation-exchange chromatography (CEX-HPLC) using non-porous 3 μm polystyrene divinylbenzene bead derivatized with carboxylic acid and elution by a gradient of up to 200 mM CaCl₂) in acetic buffer, the first subspecies elutes at about 80-85 mM CaCl₂).

21. The composition of any one of claims 1-20, wherein when the composition is subject to cation-exchange chromatography (CEX-HPLC) using non-porous 3 μm polystyrene divinylbenzene bead derivatized with carboxylic acid and elution by a gradient of up to 200 mM CaCl₂) in acetic buffer, the second subspecies elutes at about 70-75 mM CaCl₂).

22. The composition of any one of claims 1-21, wherein when the composition is subject to cation-exchange chromatography (CEX-HPLC) using non-porous 3 μm polystyrene divinylbenzene bead derivatized with carboxylic acid and elution by a gradient of up to 200 mM CaCl₂) in acetic buffer, the third subspecies elutes at about 57-58 mM CaCl₂).

23. The composition of any one of claims 1-22, wherein when the composition is subject to cation-exchange chromatography (CEX-HPLC) using non-porous 3 μm polystyrene divinylbenzene bead derivatized with carboxylic acid and elution by a gradient of up to 200 mM CaCl₂) in acetic buffer, the fourth subspecies elutes at about 180-200 mM CaCl₂).

24. The composition of any one of claims 1-23, wherein when the composition is subject to cation-exchange chromatography (CEX-HPLC) using non-porous 3 μm polystyrene divinylbenzene bead derivatized with carboxylic acid and elution by a gradient of up to 200 mM CaCl₂) in acetic buffer, the fifth subspecies elutes at about 95-105 mM CaCl₂.

25. The composition of any one of claims 1-23, wherein the second fusion protein is capable of binding to the cell surface of respiratory epithelium.

26. A pharmaceutical composition comprising the composition of any one of claims 1-25.

27. A method of releasing a composition of any one of claims 1-25 for human medical use, comprising subjecting the composition to CEX-HPLC, and determining the relative amounts of the first, second, third, fourth, and optionally fifth subspecies separated by the CEX-HPLC.

28. The method of claim 27, wherein a weight percentage of the first subspecies among the total proteins in the composition being about 50% to about 90% is indicative of the suitability of the composition for human medical use.

29. The method of claim 27 or claim 28, wherein a weight percentage of the second subspecies among the total proteins in the composition being about 15% to about 30% is indicative of the suitability of the composition for human medical use.

30. The method of any one of claims 27-29, wherein a weight percentage of the third subspecies among the total proteins in the composition being about 1% to about 15% is indicative of the suitability of the composition for human medical use.

31. The method of any one of claims 27-30, wherein a weight percentage of the fourth subspecies among the total proteins in the composition being about 0.1% to about 4% is indicative of the suitability of the composition for human medical use.

32. The method of any one of claims 27-31, wherein a weight percentage of the fifth subspecies among the total proteins in the composition being about 1% to about 10% is indicative of the suitability of the composition for human medical use.

33. The method of any one of claims 27-31, wherein the CEX-HPLC comprises using non-porous 3 μm polystyrene divinylbenzene bead derivatized with carboxylic acid and elution by a gradient of up to 200 mM CaCl₂) in acetic buffer.

34. A pharmaceutical composition released for human medical use by the method of any one of claims 27-33.

35. A method of making a pharmaceutical composition comprising the composition of any one of claims 1-26, comprising:
a) introducing a nucleic acid encoding a protein of SEQ ID NO: 1 into a bacterial host cell;
b) expressing the protein encoded by the nucleic acid in the bacterial host cell;
c) purifying the protein by chromatography to obtain a purified protein composition; and
d) assessing suitability of the purified protein composition for human medical use, wherein the assessing comprises subjecting the purified protein composition to CEX-HPLC and determining the relative amounts of the first, second, third, fourth, and optionally fifth subspecies separated by the CEX-HPLC.

36. The method of claim 35, further comprising formulating the purified protein composition to obtain a pharmaceutical composition.

37. The method of claim 36, wherein the step of formulating the purified protein composition is carried out after the step of assessing suitability of the purified protein composition.

38. The method of claim 36, wherein the step of formulating the purified protein composition is carried out before the step of assessing suitability of the purified protein composition.

39. A pharmaceutical composition made according to the method of any one of claims 35-38.

40. The pharmaceutical composition of any one of claim 26, 34, or 39, wherein the pharmaceutical composition comprises at least about 70% Trehalose by dry weight.

41. The pharmaceutical composition of any one of claim 26, 34, 39, or 40, wherein the pharmaceutical composition comprises at least about 0.2% MgSO₄ by dry weight.

42. The pharmaceutical composition of any one of claim 26, 34, or 39-41, wherein the pharmaceutical composition comprises a) about 95-98% w/w trehalose; b) about 0.2-0.4% w/w MgSO₄; c) about 0.4-0.6% w/w sodium acetate; and d) about 0.1-0.3% w/w acetic acid.

43. A pharmaceutical composition comprising: a) a fusion protein comprising a sialidase domain fused at its C-terminus to a cationic domain; b) about 95-98% w/w trehalose; c) about 0.2-0.4% w/w MgSO₄; d) about 0.4-0.6% w/w sodium acetate; and e) about 0.1-0.3% w/w acetic acid.

44. The pharmaceutical composition of any one of claim 26, 34, or 39-43, wherein the pharmaceutical composition is formulated as a lyophilized formulation.

45. The pharmaceutical composition of any one of claims 42-44, wherein the composition does not comprise histidine or CaCl₂).

46. The pharmaceutical composition of any one of claims 42-45, wherein the pharmaceutical composition comprises about 97.5% w/w Trehalose, about 0.3% w/w MgSO₄, about 0.5% w/w sodium acetate, and about 0.2% w/w acetic acid.

47. The pharmaceutical composition of any one of claims 26, 34, and 39-46, wherein the potency of the sialidase in the pharmaceutical composition is about 540-740 U/mg protein.

48. The pharmaceutical composition of any one of claims 26, 34, and 39-47, wherein the pharmaceutical composition upon reconstitution into a liquid formulation has an osmolality of about 270-330 mOsm/kg.

49. The pharmaceutical composition of any one of claims 26, 34, and 39-48, wherein the pharmaceutical composition upon reconstitution into a liquid formulation has a viscosity of about 1.27-1.39 cps.

50. The pharmaceutical composition of any one of claims 26, 34, and 39-49, wherein the pharmaceutical composition upon reconstitution into a liquid formulation has a pH of about 4.5 to about 6.5.

51. The pharmaceutical composition of any one of claims 26, 34, and 39-50, wherein at least about 95% of the proteins in the pharmaceutical composition are monomers.

52. A liquid formulation reconstituted from the pharmaceutical composition of any of claims 26, 34, and 39-51.

53. A vial comprising any one of the pharmaceutical compositions of any one of claims 26, 34, and 39-52.

54. The vial of claim 53, wherein the vial is for single use.

55. A nebulizer comprising a liquid formulation, wherein the liquid formulation is reconstituted from the pharmaceutical composition of any one of claims 26, 34, and 39-51.

56. A commercial batch comprising the pharmaceutical composition of any one of claims 26, 34, and 39-51, the vial of claim 54, or the nebulizer of claim 53.

57. A method of treating a disease in an individual comprising administering to the individual an effective amount of a pharmaceutical composition of any one of claims 26, 34, and 39-51 or a liquid formulation reconstituted from said pharmaceutical composition.


SEQUENCE LISTING (2 sequences)

SEQ ID NO: 1 - protein, 415 aa, synthetic construct
MGDHPQATPAPAPDASTELPASMSQAQHLAANTATDNYRIPAITTAPNGDLLISYDERPKDNGNGGSDAPNPNHIVQRRSTDGGKTWSAPTYIHQGTETGKKVGYSDPSYVVDHQTGTIFNFHVKSYDQGWGGSRGGTDPENRGIIQAEVSTSTDNGWTWTHRTITADITKDKPWTARFAASGQGIQIQHGPHAGRLVQQYTIRTAGGAVQAVSVYSDDHGKTWQAGTPIGTGMDENKVVELSDGSLMLNSRASDGSGFRKVAHSTDGGQTWSEPVSDKNLPDSVDNAQIIRAFPNAAPDDPRAKVLLLSHSPNPRPWSRDRGTISMSCDDGASWTTSKVFHEPFVGYTTIAVQSDGSIGLLSEDAHNGADYGGIWYRNFTMNWLGEQCGQKPAKRKKKGGKNGKNRRNRKKKNP

SEQ ID NO: 2 - protein, 414 aa, synthetic construct
GDHPQATPAPAPDASTELPASMSQAQHLAANTATDNYRIPAITTAPNGDLLISYDERPKDNGNGGSDAPNPNHIVQRRSTDGGKTWSAPTYIHQGTETGKKVGYSDPSYVVDHQTGTIFNFHVKSYDQGWGGSRGGTDPENRGIIQAEVSTSTDNGWTWTHRTITADITKDKPWTARFAASGQGIQIQHGPHAGRLVQQYTIRTAGGAVQAVSVYSDDHGKTWQAGTPIGTGMDENKVVELSDGSLMLNSRASDGSGFRKVAHSTDGGQTWSEPVSDKNLPDSVDNAQIIRAFPNAAPDDPRAKVLLLSHSPNPRPWSRDRGTISMSCDDGASWTTSKVFHEPFVGYTTIAVQSDGSIGLLSEDAHNGADYGGIWYRNFTMNWLGEQCGQKPAKRKKKGGKNGKNRRNRKKKNP